(19) United States
(12) Patent Application Publication
(10) Pub. No.: US 2006/0275769 A1
(43) Pub. Date: Dec. 7, 2006

(54) Methods of treatment and diagnosis of kaposi's sarcoma (ks) and ks related diseases

(75) Inventors: Ashlee Moses, Portland, OR (US); Klaus Fruh, Portland, OR (US); Jeffrey S. King, Portland, OR (US); James B. Hicks, Portland, OR (US); Camilo Raggo, Portland, OR (US); Jay Nelson, Portland, OR (US)

Correspondence Address: DAVIS WRIGHT TREMAINE, LLP, 2600 CENTURY SQUARE, 1501 FOURTH AVENUE, SEATTLE, WA, 98101-1688, US

(73) Assignee: Oregon Health & Science University, Portland, OR (US)

(21) Appl. No.: 10/541,598

(22) PCT Filed: Jan. 6, 2004
(86) PCT No.: PCT/US04/00320

§ 371 Date: Jul. 3, 2006

Related U.S. Application Data
(60) Provisional application No. 60/438,343, filed on Jan. 6, 2003.
(60) Provisional application No. 60/473,246, filed on May 22, 2003.

(51) Int. Cl. C12Q 1/70; C12Q 1/68

(52) U.S. Cl. 435/6; 435/5

(57) ABSTRACT

The present invention uses gene expression profiling, and gene silencing methods to identify and provide a plurality of ‘validated’ KSHV-induced cellular gene sequences and pathways useful as targets for modulation of KSHV-mediated effects on cellular proliferation and phenotype (e.g., cancer) associated with latent and lytic phases of the Kaposi's sarcoma-associated herpesvirus (KSHV; Human herpesvirus 8; HHV8) life cycle. Particular embodiments provide therapeutic compositions, and methods for modulation of KSHV infection or KSHV-mediated effects on cellular proliferation and phenotype, comprising inhibition of KSHV-induced gene sequences. Additional embodiments provide screening assays for compounds useful to modulate KSHV infection or KSHV-mediated effects on cellular proliferation and phenotype. Further embodiments provide diagnostic and/or prognostic assays for KSHV infection.

### FIELD OF THE INVENTION

The present invention relates to the identification and use of modulators of KSHV-induced cellular gene expression. Preferred modulators are inhibitors capable of reducing the expression of KSHV-induced genes, reducing or preventing the expression of mRNA from KSHV-induced genes, or reducing the biological activity of corresponding KSHV-induced cellular gene products. The invention provides therapeutic methods, diagnostic methods and compositions useful for the treatment of Kaposi's sarcoma (KS) and related cancers. Particular embodiments also provide drug candidate screening assays. The present invention uses nucleic acid microarrays and gene expression profiling, along with antisense oligonucleotide methods to identify and validate, respectively, therapeutically useful gene targets that are regulated upon KSHV infection of endothelial cells.

### BACKGROUND

Kaposi's Sarcoma (KS) is the most frequent malignancy afflicting AIDS patients.

KSHV (or human herpesvirus 8, HHV8) is consistently associated with all epidemiologic forms of KS and is recognized as the etiologic agent of the disease. KSHV infects the spindle-shaped cells that characterize the tumor as well as the corresponding lesional endothelial cell precursors, and infiltrating leukocytes. The tumor lesion is characterized by abnormal vascularization and extensive extravasation of inflammatory cells and erythrocytes. The majority of cells harbor the KSHV genome in a latent form, with a small percentage entering a lytic cycle to produce infectious virus.

Various KSHV genes are known to be capable of deregulating cellular growth, and some of these bear homology to human oncogenes, growth factors, etc., while others are unique (see e.g., Moses et al., J. Virol. 76:8383-8399, 2002). Nonetheless, relatively little is known about the influence of viral gene expression on specific cellular gene profiles, or about how such virus-cell interactions contribute to tumorigenesis. Viral gene expression patterns appear to be tumor or stage specific.

Spindle cell formation can be replicated in vitro by infection of permissive, human dermal microvascular endothelial cells (DMVEC) with KSHV (Moses et al., J. Virol. 73:6892-6902, 1999). Infection of DMVEC with KSHV results in phenotypic alteration, including spindle cell formation, loss of contact inhibition and angiogenesis in soft agar. Thus, KSHV-DMVEC interactions provide an excellent in vitro model system for KS lesion formation in vivo, and provide a means to identify those cellular gene sequences regulated in response to KSHV infection.

However, additional methods and studies are needed to distinguish, from among those KSHV-regulated cellular gene sequences, those actually required for KSHV-induced proliferative and phenotypic/developmental changes and which could therefore provide validated intervention targets for the inhibition of KSHV-induced cellular phenomena and the treatment of KSHV-induced hyperproliferative disorders such as cancer. There is a need in the art for such validated targets, and for compositions and methods to affect them.

### SUMMARY OF THE INVENTION

Nucleic acid microarray techniques were used in combination with KSHV-infected dermal microvascular endothelial cells (DMVEC) to identify and ‘validate’ cellular genes and pathways useful in modulating latent and lytic phases of the life cycle of Kaposi's sarcoma-associated herpesvirus (KSHV; Human herpesvirus 8; HHV8). The present Examples show for the first time that modulators of the expression of particular validated KSHV-induced cellular gene targets a resuitable a gents for treating KSHV-related cancer and hyperplastic/neoplastic conditions.

The present invention provides modulators of KSHV-induced gene expression including, but are not limited to antisense molecules, ribozymes, antibodies or antibody fragments, proteins or polypeptides as well as small molecules. The inventive modulators are useful for reducing the expression of KSHV-induced genes, reducing or preventing the expression of mRNA from KSHV-induced genes, or reducing the biological activity of corresponding KSHV-induced cellular gene products. Preferably, the inventive modulators are directed to one or more validated KSHV-induced gene targets, the expression of which is required, at least to some extent, for KSHV-mediated effects on cellular proliferation and phenotype.

Particular embodiments of the present invention provide therapeutic methods and compositions for modulation of KSHV infection comprising use of inventive modulators for inhibition of the expression of KSHV-induced genes, reducing or preventing the expression of mRNA from KSHV-induced genes, or reducing the biological activity of corresponding KSHV-induced cellular gene products.

Preferred inventive modulators are oligonucleotides, such as antisense molecules, siRNA, or ribozymes, to target and modulate the expression of polynucleotides (e.g., mRNA) comprising KSHV-induced gene sequences.

Preferred antisense molecules or the complements thereof comprise at least 10, 15, 20 or 25 consecutive complementary nucleotides of, or hybridize under stringent or highly stringent conditions to at least one of the nucleic acid sequences from the group consisting of SEQ ID NO:1 (cDNA for RDC1; GPCR RDC1), SEQ ID NO:3 (cDNA for IGFBP-2; insulin-like growth factor binding protein 2), SEQ ID NO:5 (cDNA for FLJ14103 protein), SEQ ID NO:7 (cDNA for KIAA0367 protein), SEQ ID NO:9 (cDNA for Neuritin), SEQ ID NO:11 (cDNA for INSR; insulin receptor), SEQ ID NO:13 (cDNA for KIT; c-kit), SEQ ID NO:25 (LOX cDNA for lysyl oxidase preprotein); SEQ ID NO:27 (NOV cDNA for nov precursor), and SEQ ID NO:29 (ANGPTL2 cDNA for angiopoietin-like 2 precursor). Preferably, such antisense molecules are PMO (phosphorodiamidate morpholino Oligomers) antisense molecules.

Preferred compositions comprise one or more of such modulators or preferred modulators, along with a pharmaceutically acceptable carrier or diluent.

Additional embodiments provide screening assays for compounds useful to modulate KSHV infection.

Further embodiments provide diagnostic or prognostic assays for KSHV infection.

### BRIEF DESCRIPTION OF THE DRAWINGS

FIG. 1A shows dermal microvascular endothelial cells (DMVECs) that are uninfected (“Mock”) (left-most panel), 1-week post-infection (central panel), or 4-weeks post-infection (right-most panel). The beginning of characteristic spindle cell formation in DMVEC cells can be seen 1-week post-infection with KSHV, and substantially progresses through 4 weeks post-infection.

FIG. 1B shows red fluorescent staining of latent KSHV infected DMVEC cells (“ORF7,” left-most panel), green fluorescent staining of lytic KSHV-infected DMVEC cells (“B-ORF59,” central panel), and green fluorescent staining of lytic KSHV-infected DMVEC cells enhanced with PMA (“ORF59+PMA,” right-most panel).

FIG. 1C shows the beginning of foci formation in KSHV-infected DMVEC at 1-week post infection (“KSHV 1 week,” left-most panel), progression of foci formation at 4-weeks post infection (“KSHV 4 weeks,” central panel), and KSHV-infected DMVECs growing in soft agar as a result of the acquisition of anchorage-independent growth (“KSHV Agar,” right-most panel).

FIG. 2 shows a pie-type chart for functional group assignment (described under “EXAMPLE 2” below, based on art-available information) of genes having altered expression in DMVEC in response to KSHV infection.

FIG. 3A shows that treatment with c-Kit PMO antisense (SEQ ID NO:21) resulted in restoring contact-inhibited growth of KSHV-infected DMVECs. Specifically, FIG. 3A (upper-left panel “A”) shows that during the week of post-loading culture, Untreated and control EPEI-treated KSHV-infected DMVECs exhibited loss of contact inhibition, and displayed the capacity to grow in disorganized, multi-layered foci that were evident by day 6 post-loading (upper-left panels “A” and “B,” respectively). By contrast, KSHV-infected DMVECs loaded with c-Kit-specific antisense PMO oligonucleotides (+EPEI) did not develop foci, and maintained a quiescent contact-inhibited monolayer (lower-left panel “C”).

FIG. 3B shows evidence that despite expression in some cells of c-kit protein (red fluorescent staining), the cell cultures treated (loaded) with c-Kit antisense PMO oligomer (SEQ ID NO:) (green fluorescent staining) did not progress to spindle cell and foci formation (e.g., see phase contrast images of FIG. 3A, lower-left panel “C”).

FIGS. 4A, 4B, 4C and 4D show representative fields of KSHV-infected DMVEC treated with various gene-specific PMO antisense oligonucleotides as indicated, and visualized by CD31 staining: 100% proliferation control (no PMO oligonucleotides) (FIG. 4A); RDC-1-specific PMO antisense oligonucleotides, resulting in 43% growth inhibition and full phenotypic inhibition (FIG. 4B); KIAA0367-specific PMO antisense oligonucleotides, resulting in 28% growth inhibition and intermediate phenotypic inhibition (FIG. 4C); and MFAP-specific PMO antisense oligonucleotides, resulting in 11% growth inhibition and no phenotypic inhibition (FIG. 4D). According to the present invention, the extent of PMO-mediated inhibition of KSHV-induced proliferation (% growth inhibition) correlates with the corresponding phenotype inhibition values (full, intermediate and no inhibition).

### DETAILED DESCRIPTION OF THE INVENTION

Identification OF KSHV-Regulated Genes and Pathways, Validation of Same as Therapeutic Targets, and Provision of Therapeutic Modulators

Overview

The present invention uses gene expression profiling, and gene silencing methods to identify and provide a plurality of ‘validated’ KSHV-induced cellular gene sequences and pathways useful as targets for modulation of KSHV-mediated effects on cellular proliferation and phenotype (e.g., cancer) associated with latent and lytic phases of the Kaposi's sarcoma-associated herpesvirus (KSHV; Human herpesvirus 8; HHV8) life cycle. Validated gene targets correspond to those KSHV-induced gene sequences the expression of which is required, at least to some extent, for KSHV-mediated effects on cellular proliferation and phenotype. Inventive modulators of validated targets are agents that act by inhibiting the expression of validated KSHV-induced genes, by reducing or preventing the expression of mRNA from validated KSHV-induced genes, or by reducing the biological activity of corresponding KSHV-induced cellular gene products. Inventive modulators of KSHV-induced gene expression include, but are not limited to antisense molecules, siRNA agents, ribozymes, antibodies or antibody fragments, proteins or polypeptides as well as small molecules.

Definitions

The term “siRNA” or “RNAi” refers to small interfering RNA as is known in the art (see e.g.: U.S. Pat. No. 6,506,559; Milhavet et al., Pharmacological Reviews 55:629-648, 2003; and Gitlin et al., J. Virol. 77:7159-7165, 2003; incorporated herein by reference).

The term “DMVEC” refers to human dermal microvascular endothelial cells.

Soft agar model system for in vivo KSHV-related cancer. Inventive KSHV-related therapeutic targets were identified by the use of a soft agar-based primary dermal microvascular endothelial cell (DMVEC) growth and differentiation assay system, which is an art-recognized model system for cancer in vivo (e.g., Tomkowicz, K et al., DNA Cell Biol. 21:151, 2002 (use of soft agar assays system to demonstrate transformation with KSHV kaposin protein); Saucier et al., Oncogene 21:1800, 2002 (use of soft agar assays system to demonstrate transformation with Met RTK protein); and see also Chernicky, C L, Mol. Pathol. 55:102, 2002 (use of inhibition of colony formation in soft agar as validation for siRNS inhibition of a tumor growth factor); and EXAMPLE 1 below). In the soft agar system, KSHV-infected DMEC display various hallmarks of KSHV-related in vivo cancer, including, but not limited to anchorage-independent growth and spindle cell formation. Significantly, inventive modulators were shown to either inhibit or cause reversion of cancer phenotype (e.g., inhibits formation of spindle cells, or causes reversion of the spindle cells phenotype), and/or to inhibit anchorage-independent growth (EXAMPLES 2 and 3, below).

Identification of KSHV-induced cellular genes using microarrays. Cellular genes involved in the transformed phenotype caused by latent infection with KSHV were identified by using DNA microarrays to examine the differential gene expression profiles of primary dermal microvascular endothelial cells (DMVEC) before and after KSHV-infection. Such microarray technology is well known in the art (see, e.g., Moses et al., J. Virol. 76:8383-8399, 2002; WO 02/10339 A2, published 7 Feb. 2002; Salunga et al., In M. Schena (ed.), DNA microarrays, A practical approach; Oxford Press, Oxford, United Kingdom, 1999; and see Simmen et al., Proc. Natl. Acad. Sci. USA 98:7140-7145, 2001; all of which are incorporated by reference herein in their entirety), and can be performed using commercially available arrays (e.g., Affymetrix U133A, U133B and U95A GeneChip® arrays) (Affymetrix, Santa Clara, Calif.). The Human Genome U133 (HG-U133) set, consists of two GeneChip® arrays, and contains almost 45,000 probe sets representing more than 39,000 transcripts derived from approximately 33,000 well-substantiated human genes (Affymetrix technical information). The set design uses sequences selected from GenBank®, dbEST, and RefSeq (Id).

Specifically, as described in detail under EXAMPLE 2 herein, nucleic acid microarray technology was used for gene expression profiling of KSHV-infected DMVEC, relative to non-infected control cells, to identify cellular genes whose expression is regulated by KSHV. Each of the DMVEC infected/uninfected sample comparisons resulted in approximately 480 probe sets with increased expression, with 316 probe sets that showed increased expression in duplicate infections. There were 390 probes sets that showed decreased expression in duplicate, out of approximately 600 probe sets that were down in individual experiments (EXAMPLE 2). The 706 probes sets identified with significant changes in expression correspond to 580 unique gene sequences.

Validation of therapeutic targets by gene silencing using gene-specific PMO antisense compounds. Additionally, particular KSHV-regulated or KSHV-induced gene sequences were identified as validated therapeutic targets by specific gene silencing using PMO (phosphorodiamidate morpholino Oligomers) antisense oligonucleotide inhibition in combination with measuring the effects of such gene silencing using cellular differentiation (EXAMPLE 3 below, at TABLE 2) or cellular proliferation assays (EXAMPLE 3 below, at TABLE 4). Silencing of such genes precluded progression into the KSHV-transformed phenotype when silencing occurred prior to transformation, or induced reversion to the normal state when silencing occurred after induction of the transformed state (EXAMPLE 3 below, at TABLE 2).

Therapeutic utility. According to the present invention, PMO-mediated gene silencing using the soft agar growth/differentiation system not only provides validation of therapeutically-significant targets, but also provides gene-specific modulators of KSHV-induced cellular gene expression that have therapeutic utility. PMOs (see, e.g., Summerton, et al., Antisense Nucleic Acid Drug Dev. 7:63-70, 1997; and Summerton & Weller, Antisense Nucleic Acid Drug Dev. 7:187-95, 1997) represent a class of art-recognized antisense drugs for treating various diseases, including cancer. For example, Arora et al. (J. Pharmaceutical Sciences 91:1009-1018, 2002) demonstrated that oral administration of c-myc-specific and CYP3A2-specific PMOs inhibited c-myc and CYP3A2 gene expression, respectively, in rat liver by an antisense mechanism of action. Likewise, Devi G. R. (Current Opinion in Molecular Therapeutics 4:138-148, 2002) discusses treatment of prostate cancer with various PMO therapeutic agents).

Likewise, siRNA” or “RNAi” agents are emerging as a new class of art-recognized drugs (see e.g.: U.S. Pat. No. 6,506,559; Milhavet et al., Pharmacological Reviews 55:629-648, 2003; and Gitlin et al., J. Virol. 77:7159-7165, 2003; incorporated herein by reference).

Accordingly, the present invention provides therapeutic compositions, and methods for modulation of KSH infection, comprising inhibition of KSHV-induced gene expression (e.g., inhibition of the expression of validated KSHV-induced genes, reducing or preventing the expression of mRNA from validated KSHV-induced genes, or reducing the biological activity of corresponding KSHV-induced cellular gene products).

Additional embodiments provide screening assays for compounds useful to modulate KSHV infection.

Further embodiments provide diagnostic or prognostic assays for KSHV infection.

Preferred Inventive Modulators. Compositions, Utilities and Expression Vectors

Modulators of KSHV-induced gene expression. Particular embodiments provide modulators of KSHV-induced cellular gene expression. Preferably, inventive modulators are directed to one or more validated KSHV-induced cellular gene targets, the expression of which is required, at least to some extent, for KSHV-mediated effects on cellular proliferation and phenotype.

Inventive modulators include, but are not limited to, antisense molecules, ribozymes, antibodies or antibody fragments, proteins or polypeptides as well as small molecules. Particular KSHV-induced gene expression modulators, such as gene-specific antisense and ribozyme molecules, and antibodies and epitope-binding fragments thereof, are inhibitors of KSHV-induced gene expression, or of the biological activity of proteins encoded thereby.

Preferably, inventive antisense molecules are oligonucleotides of about 10 to 35 nucleotides in length that are targeted to a nucleic acid molecule corresponding to a KSHV-induced gene sequence, wherein the antisense molecule inhibits the expression of at least one KSHV-induced gene sequence. Antisense compounds useful to practice the invention include oligonucleotides containing art-recognized modified backbones or non-natural internucleoside linkages, modified sugar moieties, or modified nucleobases.

Preferred antisense molecules or the complements thereof comprise at least 10, at least 15, at least 20 or at least 25, and preferably less than about 35 consecutive complementary nucleotides of, or hybridize under stringent or highly stringent conditions to at least one of the nucleic acid sequences from the group consisting of SEQ ID NO:1 (cDNA for RDC1; GPCR RDC1), SEQ ID NO:3 (cDNA for IGFBP-2; insulin-like growth factor binding protein 2), SEQ ID NO:5 (cDNA for FLJ14103 protein), SEQ ID NO:7 (cDNA for KIAA0367 protein), SEQ ID NO:9 (cDNA for Neuritin), SEQ ID NO:11 (cDNA for INSR; insulin receptor), SEQ ID NO:13 (cDNA for KIT; c-kit), SEQ ID NO:25 (LOX cDNA for lysyl oxidase preprotein); SEQ ID NO:27 (NOV cDNA for nov precursor), and SEQ ID NO:29 (ANGPTL2 cDNA for angiopoietin-like 2 precursor). Preferably, such antisense molecules are PMO (phosphorodiamidate morpholino Oligomers) antisense molecules.

Thus, the present invention includes nucleic acids that hybridize under stringent hybridization conditions, as defined below, to all or a portion of the validated KHSV-induced cellular gene sequences represented by the cDNA sequences of SEQ ID NOS: 1, 3, 5, 7, 9, 11, 13, 25, 27 and 29, or the complements thereof. The hybridizing portion of the hybridizing nucleic acids is typically at least 10, 15, 20, 25, 30 or 35 nucleotides in length. Preferably, the hybridizing portion of the hybridizing nucleic acid is at least 80%, at least 95%, or at least 98% identical to the sequence of a portion or all of the cDNA sequences of SEQ ID NOs: 1, 3, 5, 7, 9, 11, 13, 25, 27 and 29, or to the complements thereof.

Hybridizing nucleic acids of the type described herein can be used, for example, as an inventive therapeutic modulator of KSHV-induced gene expression, a cloning probe, a primer (e.g., a PCR primer), or a diagnostic and/or prognostic probe or primer. Preferably, hybridization of the oligonucleotide probe to a nucleic acid sample is performed under stringent conditions. Nucleic acid duplex or hybrid stability is expressed as the melting temperature or Tm, which is the temperature at which a probe dissociates from a target DNA. This melting temperature is used to define the required stringency conditions.

For sequences that are related and substantially identical to the probe, rather than identical, it is useful to first establish the lowest temperature at which only homologous hybridization occurs with a particular concentration of salt (e.g., SSC or SSPE). Then, assuming that 1% mismatching results in a 1° C. decrease in the Tm, the temperature of the final wash in the hybridization reaction is reduced accordingly (for example, if sequences having >95% identity with the probe are sought, the final wash temperature is decreased by 5° C.). In practice, the change in Tm can be between 0.5° C. and 1.5° C. per 1% mismatch.

Stringent conditions, as defined herein, involve hybridizing at 68° C. in 5×SSC/5× Denhardt's solution/1.0% SDS, and washing in 0.2×SSC/0.1% SDS at room temperature, or involve the art-recognized equivalent thereof. Moderately stringent conditions, as defined herein, involve including washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons; N.Y.) at Unit 2.10.

Antisense molecules preferably comprise at least 20, or at least 25, and preferably less than about 35 consecutive complementary nucleotides of, or hybridize under stringent conditions to at least one of the nucleic acid sequences from the group consisting of SEQ ID NOS:1, 3, 5, 7, 9, 11, 13, 25, 27 and 29. Preferably, such antisense molecules are PMO antisense molecules. Preferred representative antisense molecules are provided herein as:

SEQ ID NO:15

| | |
|---|---|
| (RDC-1) | 5′-GAAGAGATGCAGATCCATCGTTCTG-3′); |

SEQ ID NO:16

| | |
|---|---|
| (IGFBP2) | 5′-GGCAGCCCACTCTCTCGGCAGCATG-3′); |

SEQ ID NO:17

| | |
|---|---|
| (FLJ14103) | 5′-GGCTCCATCTTGGGCTCTGGGCTCC-3′); |

SEQ ID NO:18

| | |
|---|---|
| (KIAA0367) | 5′-GTCAGTTTACTCATGTCATCTATTG-3′); |

SEQ ID NO:19

| | |
|---|---|
| (Neuritin) | 5′-TTAACTCCCATCCTACGTTTAGTCA-3′); |

SEQ ID NO:20

| | |
|---|---|
| (INSR) | 5′-GGGTCTCCTCGGATCAGGCGCG-3′); |

SEQ ID NO:21

| | |
|---|---|
| (KIT) | 5′-CGCCTCTCATCGCGGTAGCTGCG-3′); |

SEQ ID NO:31

| | |
|---|---|
| (LOX) | 5′-GGAGCACGGTCCAGGCGAAGCGCAT-3′); |

SEQ ID NO:32

| | |
|---|---|
| (NOV) | 5′-AGCTCGTGCTCTGCACACTCTGCAT-3′); |
| and | |

SEQ ID NO:33

| | |
|---|---|
| (ANGPTL2) | 5′-AGCATGTCACGCACAGTGGCCTCAT-3′). |

Preferably, these antisense molecules are PMO antisense molecules.

Even more preferably, representative antisense molecules are provided herein as SEQ ID NOS:15, 16, 17, 19, 21, 31, 32 and 33, and these antisense molecules are preferably PMO antisense molecules.

The invention further provides a ribozyme capable of specifically cleaving at least one RNA specific to RDC-1, IGFBP2, FLJ14103, KIAA0367, Neuritin, INSR, KIT, LOX, NOV and ANGPTL2, and a pharmaceutical composition comprising the ribozyme.

The invention also provides small molecule modulators of KSHV-induced gene expression, wherein particular modulators are inhibitors capable of reducing the expression of at least one KSHV-induced genes, reducing or preventing the expression of mRNA from at least one KSHV-induced gene, or reducing the biological activity of at least one KSHV-induced gene product. Preferably, the KSHV-induced gene is selected from the group consisting of RDC-1, IGFBP2, FLJ14103, KIAA0367, Neuritin, INSR, KIT, LOX, NOV and ANGPTL2.

Compositions. Further embodiments provide compositions that comprise one or more modulators of KSHV-induced gene expression (or modulators of biological activity of KSHV-induced gene products) in a pharmaceutically acceptable carrier or diluent.

Particular embodiments provide a pharmaceutical composition for inhibiting KSHV-induced gene expression, comprising an antisense oligonucleotide according to the invention in a mixture with a pharmaceutically acceptable carrier or diluent.

Further provided is a composition comprising a therapeutically effective amount of an inhibitor of a KSHV-induced gene product (e.g., protein) in a pharmaceutically acceptable carrier. In certain embodiments, the composition comprises two or more KSHV-induced gene product inhibitors. Preferably, the KSHV-induced gene product is selected from the group consisting of SEQ ID NOS:2, 4, 6, 8, 10, 12, 14, 26, 28 and 30, and combinations thereof, corresponding to RDC-1, IGFBP2, FLJ14103, KIAA0367, Neuritin, INSR, KIT, Lysyl Oxidase precursor (LOX), nov precursor (NOV), angiopoietin-like 2 precursor (ANGPTL2), and combinations thereof, respectively.

In particular composition embodiments, the KSHV-induced gene inhibitor is an antisense molecule, and in specific embodiments the antisense molecule or the complement thereof comprises at least 10, 15, 20 or 25 consecutive nucleic acids of, or hybridizes under stringent conditions to at least one of the nucleic acid sequences from the group consisting of SEQ ID NOS:1, 3, 5, 7, 9, 11, 13, 25, 27 and 29. Preferably, such antisense molecules are PMO antisense molecules. Preferably, the antisense molecule comprises a nucleic acid sequence selected from the group consisting of SEQ ID NOS:15-21 and SEQ ID NOS:31-33. Preferably, the antisense molecules comprises a nucleic acid sequence selected from the group consisting of SEQ ID NOS:15, 16, 17, 19, 21, 31, 32 and 33.

Methods and uses. Particular embodiments of the present invention provide methods of modulating KSHV-induced gene expression or biological activity of KSHV-induced gene products in KSHV-infected cells.

The invention provides a method of inhibiting the expression of KSHV-induced cellular genes in human cells or tissues comprising contacting the cells or tissues in vivo (also ex vivo, or in vitro) with an antisense compound or a ribozyme of 10 to 35 nucleotides in length targeted to a nucleic acid molecule encoding a KSHV-induced gene product so that expression of the human

KSHV-induced gene product is inhibited. Preferably, the KSHV-induced gene is selected from the group consisting of RDC-1 (GPCR RDC1), IGFBP2 (insulin-like growth factor binding protein 2), FLJ14103, KIAA0367, Neuritin, INSR (insulin receptor), KIT, Lysyl Oxidase precursor (LOX), nov precursor (NOV), angiopoietin-like 2 precursor (ANGPTL2), and combinations thereof. Preferably, the antisense compounds are PMOs.

The invention additionally provides a method of modulating growth of cancer cells comprising contacting the cancer cells in vivo (also ex vivo, or in vitro) with an inventive antisense compound or ribozyme of 10 to 35 nucleotides in length targeted to a nucleic acid molecule encoding a KSHV-induced gene product so that expression of the human KSHV-induced gene product is inhibited.

The invention provides for the use of a modulator of KSHV-induced gene expression according to the invention to prepare a medicament for modulating cell proliferation and/or phenotype.

Additional embodiments provide a method of inhibiting KSHV-induced gene expression or encoded biological activity in a mammalian cell, comprising administering to the cell an inhibitor of KSHV-induced gene expression (or of encoded biological activity), and in a specific embodiment of the method, the inhibitor is a target gene-specific antisense molecule.

Preferably, the antisense molecule is a PMO antisense molecule. Preferably, the antisense molecules comprises a nucleic acid sequence selected from the group consisting of SEQ ID NOS:15-21 and SEQ ID NOS:31-33.

The invention also provides a method of inhibiting KSHV-induced gene expression in a subject, comprising administering to said subject, in a pharmaceutically effective vehicle, an amount of an antisense oligonucleotide which is effective to specifically hybridize to all or part of a selected target nucleic acid sequence derived from said KSHV-induced gene. In preferred embodiments of this method, the target-specific antisense oligonucleotide is selected from the group consisting of SEQ ID NOS:15-21 and SEQ ID NOS:31-33. Preferably, the antisense oligonucleotide is selected from the group consisting of SEQ ID NOS:15, 16, 17, 19, 21, 31, 32 and 33. Preferably the antisense oligonucleotides are PMO antisense compounds.

The invention further provides a method of treating KSHV-related neoplastic disease, comprising administering to a mammalian cell a modulator of KSHV-induced gene expression such that the neoplastic disease is reduced in severity.

As discussed herein below, additional embodiments provide screening assays for identification of compounds useful to modulate KSHV infection, comprising: contacting KSHV-infected cells with a test a gent; measuring, using a suitable assay, expression of at least one validated KSHV-induced cellular gene sequence; and determining whether the test agent inhibits said validated gene expression relative to control cells not contacted with the test agent, whereby agents that inhibit said validated gene expression are identified as compounds useful to modulate KSHV infection.

Preferably, expression of at least one validated KSHV-induced cellular gene sequence is expression of respective mRNA, or expression of the protein encoded thereby.

Preferably, the at least one validated KSHV-induced cellular gene sequence is selected from the cDNA and protein sequence group consisting of RDC-1, IGFBP2, FLJ14103, KIAA0367, Neuritin, INSR, KIT, Lysyl Oxidase precursor (LOX), nov precursor (NOV), angiopoietin-like 2 precursor (ANGPTL2), and combinations thereof (ie., consisting of SEQ ID NOS:1-14 and SEQ ID NOS:25-30).

Preferably, agents that inhibit said validated gene expression are further tested for the ability to modulate KSHV-mediated effects on cellular proliferation and/or phenotype.

Further embodiments provide diagnostic or prognostic assays for KSHV infection comprising: obtaining a cell sample from a subject suspected of having KSHV; measuring expression of at least one validated KSHV-inducible cellular gene sequence; and determining whether expression of the at least one validated gene is induced relative to non-KSHV-infected control cells, whereby a diagnosis is afforded.

Preferably, the at least one validated KSHV-inducible cellular gene is selected from the cDNA and protein sequence group consisting of RDC-1, IGFBP2, FLJ14103, KIAA0367, Neuritin, INSR, KIT, Lysyl Oxidase precursor (LOX), nov precursor (NOV), angiopoietin-like 2 precursor (ANGPTL2), and combinations thereof (i.e., consisting of SEQ ID NOS:1-14 and SEQ ID NOS:25-30).

Preferably, measuring said expression is of two or more validated KSHV-inducible cellular gene sequences. Preferably, measurement of said expression is by use of high-throughput microarray methods.

Polynucleotides and expression vectors. Particular embodiments provide an isolated polynucleotide with a sequence comprising a transcriptional initiation region and a sequence encoding a KSHV-induced gene-specific antisense oligonucleotide at least 10, 15, 20 or 25 nucleotides in length, and a recombinant vector comprising this polynucleotide (e.g., expression vector). Preferably, the antisense oligonucleotide of said polynucleotide comprises a sequence selected from the group consisting of SEQ ID NOS:15-21 and SEQ ID NOS:31-33. Preferably, the transcriptional initiation region is a strong constitutively expressed mammalian pol III- or pol II-specific promoter, or a viral promoter.

Additional and Preferred Oligonucleotide Modulators

Included within the scope of the invention are oligonucleotides capable of hybridizing with KSHV-induced gene DNA or RNA, referred to herein as the ‘target’ polynucleotide. An oligonucleotide need not be 100% complementary to the target polynucleotide, as long as specific hybridization is achieved. The degree of hybridization to be achieved is that which interferes with the normal function of the target polynucleotide, be it transcription, translation, pairing with a complementary sequence, or binding with another biological component such as a protein. An antisense oligonucleotide, including a preferred PMO antisense oligonucleotide, can interfere with DNA replication and transcription, and it can interfere with RNA translocation, translation, splicing, and catalytic activity.

The invention includes within its scope any oligonucleotide of about 10 to about 35 nucleotides in length, including variations as described herein, wherein the oligonucleotide hybridizes to a KHSV-induced target sequence, including DNA or mRNA, such that an effect on the normal function of the polynucleotide is achieved. The oligonucleotide can be, for example, 10, 15, 20, 22, 23, 25, 30 or 35 nucleotides in length. Oligonucleotides larger than 35 nucleotides are also contemplated within the scope of the present invention, and may for example, correspond in length to a complete target cDNA (i.e., mRNA) sequence, or to a significant or substantial portion thereof.

Antisense oligonucleotides. As described above, preferred antisense molecules are represented by SEQ ID NOS:15-21 and SEQ ID NOS:31-33.

Examples of representative preferred antisense compounds useful in the invention are based on SEQ ID NOS:1, 3, 5, 7, 9, 11, 13, 25, 27, 29, and SEQ ID NOS:15-21 and 31-33, and include oligonucleotides containing modified backbones or non-natural internucleoside linkages. Oligonucleotides having modified backbones include those retaining a phosphorus atom in the backbone, and those that do not have a phosphorus atom in the backbone.

Preferred modified oligonucleotide backbones include phosphorothioates or phosphorodithioate, chiral phosphorothioates, phosphotriesters and alkyl phosphotriesters, aminoalkylphosphotriesters, methyl and other alkyl phosphonates including methylphosphonates, 3′-alkylene phosphonates and chiral phosphonates, phosphinates, phosphoroamidates or phosphordiamidates, including 3′-amino phosphoroamidate and aminoalkylphosphoroamidates, and phosphorodiamidate morpholino oligomers (PMOs), thiophosphoroamidates, phosphoramidothioates, thioalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

The antisense oligonucleotide may also comprise at least one modified sugar moiety selected from the group including, but not limited to arabinose, 2-fluoroarabinose, xylulose, hexose and 2′-O-methyl sugar moieties.

The antisense oligonucleotide may comprise at least one modified base moiety which is selected from the group including, but not limited to 5-fluorouracil, 5-bromouracil, 5-chlorouracil, 5-iodouracil, hypoxanthine, xanthine, 4-acetylcytosine, 5-carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine (see also U.S. Pat. No. 5,958,773 and patents disclosed therein).

Examples of inventive antisense oligonucleotides of length X (in nucleotides), as indicated by polynucleotide positions with reference to, e.g., SEQ ID NO:1, include those corresponding to sets of consecutively overlapping oligonucleotides of length X, where the oligonucleotides within each consecutively overlapping set (corresponding to a given X value) are defined as the finite set of Z oligonucleotides from nucleotide positions:
n to (n+(X−1));
where n=1, 2, 3, . . . (Y−(X−1));
where Y equals the length (nucleotides or base pairs) of SEQ ID NO:1 (2,035);
where X equals the common length (in nucleotides) of each oligonucleotide in the set (e.g., X=20 for a set of consecutively overlapping 20-mers); and

where the number (Z) of consecutively overlapping oligomers of length X for a given SEQ ID NO of length Y is equal to Y−(X−1). For example Z=2,035−19=2,016 for SEQ ID NO:1, where X=20.

Examples of inventive 20-mer oligonucleotides include the following set of 2,016 oligomers, indicated by polynucleotide positions with reference to SEQ ID NO:1 (RDC-1 cDNA):

1-20, 2-21, 3-22, 4-23, 5-24 . . . 2014-2033, 2015-2034 and 2016-2035.

Likewise, examples of 25-mer oligonucleotides include the following set of 2,011 oligomers, indicated by polynucleotide positions with reference to SEQ ID NO:1:

1-25, 2-26, 3-27, 4-28, 5-29 . . . 2009-2033, 2010-2034 and 2011-2035.

The present invention encompasses, for each validated target sequence (e.g., for SEQ ID NOS:1, 3, 5, 7, 9, 11, 13, 25, 27 and 29), multiple consecutively overlapping sets of oligonucleotides or modified oligonucleotides of length X, where, e.g., X=10, 20, 22, 23, 25, 30 or 35 nucleotides.

Preferred sets of such oligonucleotides or modified oligonucleotides of length X are those consecutively overlapping sets of oligomers corresponding to SEQ ID NOS:1, 3, 5, 7, 9, 11, 13, 25, 27 and 29. Included in these preferred sets are the preferred oligomers corresponding to SEQ ID NOS: 15-21 and SEQ ID NOS:31-33.

The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773. Thus, the oligonucleotide may include other appended groups such as peptides (e.g., for targeting host cell receptors in vivo), or agents facilitating or modulating transport across the cell membrane (Letsinger et al., Proc. Natl. Acad. Sci. USA 86:6553-6556, 1989; Lemaitre et al., Proc. Natl. Acad. Sci. USA 84:648-652, 1987; PCT WO88/09810, published Dec. 15, 1988) or the blood-brain barrier (PCT WO89/10134, published Apr. 25, 1988), or the nuclear membrane, and may include hybridization-triggered cleavage agents (Krol et al., BioTechniques 6:958-976, 1988) or intercalating agents (Zon, Pharm. Res. 5:539-549, 1988). To this end, the oligonucleotide may be conjugated to another molecule, e.g., a peptide, hybridization-triggered cross-linking agent, transport agent, hybridization-triggered cleavage agent, etc.

Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5,491,133, 5,565,350, 5,652,355, 5,700,922 and 5,958,773.

Preferred antisense oligonucleotides in addition to those of SEQ ID NOS:15-21 are selected by routine experimentation using, for example, assays described in the present Examples. Although the inventors are not bound by a particular mechanism of action, it is believed that the antisense oligonucleotides achieve an inhibitory effect by binding to a complementary region of the target polynucleotide within the cell using Watson-Crick base pairing. Where the target polynucleotide is RNA, experimental evidence indicates that the RNA component of the hybrid is cleaved by RNase H (Giles, R. V. et al., Nuc. Acids Res. (1995) 23:954-961; U.S. Pat. No. 6,001,653). Generally, a hybrid containing 10 base pairs is of sufficient length to serve as a substrate for RNase H. However, to achieve specificity of binding, it is preferable to use an antisense molecule of at least 17 nucleotides, as a sequence of this length is likely to be unique among human genes.

Antisense approaches comprise the design of oligonucleotides (either DNA or RNA) that are complementary to the target gene sequence (e.g., mRNA). The antisense oligonucleotides bind to the complementary mRNA transcripts and prevent translation. Absolute complementarily, although preferred, is not required. A sequence “complementary” to a portion or region of the target mRNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize depends on both the degree of complementarity and the length of the antisense nucleic acid. Generally, the longer the hybridizing nucleic acid, the more base mismatches with an RNA are accommodated without compromising stable duplex (or triplex, as the case may be) formation. One skilled in the art ascertains a tolerable degree of mismatch by use of standard procedures to determine the melting point of the hybridized complex.

As disclosed in U.S. Pat. No. 5,998,383, incorporated herein by reference, the oligonucleotide is selected such that the sequence exhibits suitable energy related characteristics important for oligonucleotide duplex formation with their complementary targets, and shows a low potential for self-dimerization or self-complementation (Anazodo et al., Biochem. Biophys. Res. Commun. (1996) 229:305-309). The computer program OLIGO (Primer Analysis Software, Version 3.4), is used to determined antisense sequence melting temperature, free energy properties, and to estimate potential self-dimer formation and self-complementarity properties. The program allows the determination of a qualitative estimation of these two parameters (potential self-dimer formation and self-complementary) and provides an indication of “no potential” or “some potential” or “essentially complete potential.” Preferably, segments of validated KSHV-induced gene sequences are selected that have estimates of no potential in these parameters. However, segments that have “some potential” in one of the categories nonetheless can have utility, and a balance of the parameters is routinely used in the selection.

While antisense nucleotides complementary to the coding region sequence of a mRNA are used in accordance with the invention, those complementary to the transcribed, untranslated region, or translational initiation site region are sometimes preferred. Oligonucleotides that are complementary to the 5′ end of the message, e.g., the 5′-untranslated sequence (up to and including the AUG initiation codon), frequently work most efficiently at inhibiting translation.

However, sequences complementary to the 3′-untranslated sequences, or other regions of mRNAs are also effective at inhibiting translation of mRNAs (see e.g., Wagner, Nature 372:333-335, 1994). In the antisense art a certain degree of routine experimentation is required to select optimal antisense molecules for particular targets. To be effective, the antisense molecule preferably is targeted to an accessible, or exposed, portion of the target RNA molecule.

Although in some cases information is available about the structure of target mRNA molecules, the current approach to inhibition using antisense is via experimentation.

Such experimentation can be performed routinely by transfecting or loading cells with an antisense oligonucleotide, followed by measurement of messenger RNA (mRNA) levels in the treated and control cells by reverse transcription of the mRNA and assaying of respective cDNA levels. Measuring the specificity of antisense activity by assaying and analyzing cDNA levels is an art-recognized method of validating antisense results. Routinely, RNA from treated and control cells is reverse-transcribed and the resulting cDNA populations are analyzed (Branch, A. D., T.I.B.S. (1998) 23:45-50).

According to the present invention, antisense efficacy can be alternately determined by measuring the biological effects on cell growth, phenotype or viability as is known in the art, and as shown in the present Examples. According to the present invention, cultures of KSHV-infected DMVEC were loaded with inventive oligonucleotides designed to target KSHV-induced gene sequences. Preferred representative antisense oligonucleotides correspond to SEQ ID NOS:15-21. The effects of such loading on cellular proliferation and/or phenotype were measured. Specifically, SEQ ID NOS:15-21 caused dramatic decreases in cell proliferation and inhibited/reverted spindle cell formation, both hallmarks of in vivo KSHV-related cancer.

Ribozymes. Modulators of KSHV-induced gene expression may be ribozymes. A ribozyme is an RNA molecule that specifically cleaves RNA substrates, such as mRNA, resulting in specific inhibition or interference with cellular gene expression. As used herein, the term ribozymes includes RNA molecules that contain antisense sequences for specific recognition, and an RNA-cleaving enzymatic activity. The catalytic strand cleaves a specific site in a target RNA at greater than stoichiometric concentration. Preferably the ribozyme is engineered so that the cleavage recognition site is located near the 5′ end of the target mRNA (i.e., to increase efficiency and minimize the intracellular accumulation of non-functional mRNA transcripts).

A wide variety of ribozymes may be utilized within the context of the present invention, including for example, the hammerhead ribozyme (for example, as described by Forster and Symons, Cell (1987) 48:211-220; Haseloff and Gerlach, Nature (1988) 328:596-600; Walbot and Bruening, Nature (1988) 334:196; Haseloff and Gerlach, Nature (1988) 334:585); the hairpin ribozyme (for example, as described by Haseloff et al., U.S. Pat. No. 5,254,678, issued Oct. 19, 1993 and Hempel et al., European Patent Publication No. 0 360 257, published Mar. 26, 1990); and Tetrahymena ribosomal RNA-based ribozymes (see Cech et al., U.S. Pat. No. 4,987,071). The Cech-type ribozymes have an eight-base pair active site that hybridizes to a target RNA sequence whereafter cleavage of the target RNA takes place. Ribozymes of the present invention typically consist of RNA, but may also be composed of DNA, nucleic acid analogs (e.g., phosphorothioates), or chimerics thereof (e.g., DNA/RNA/RNA).

Ribozymes can be targeted to any RNA transcript and can catalytically cleave such transcripts (see, e.g., U.S. Pat. No. 5,272,262; U.S. Pat. No. 5,144,019; and U.S. Pat. Nos. 5,168,053, 5,180,818, 5,116,742 and 5,093,246 to Cech et al.). According to certain embodiments of the invention, any such KSHV-induced gene sequence-specific ribozyme, or a nucleic acid encoding such a ribozyme, may be delivered to a host cell to effect inhibition of KSHV-induced gene expression. Ribozymes and the like may therefore be delivered to the host cells by DNA encoding the ribozyme linked to a eukaryotic promoter (e.g., a strong constitutively expressed pol III- or pol II-specific promoter), or a eukaryotic viral promoter, such that upon introduction into the nucleus, the ribozyme will be directly transcribed.

Triple-helix formation. Alternatively, validated KSHV-induced gene expression can be reduced by targeting deoxyribonucleotide sequences complementary to the regulatory region of the target gene (e.g., respective promoter and/or enhancers) to form triple helical structures that prevent transcription of the target gene (see, e.g., Helen, Anticancer Drug Des., 6:569-84, 1991;

Helene et al., Ann, N.Y. Acad. Sci., 660:27-36, 1992; and Maher, Bioassays 14:807-15, 1992).

siRNA. The invention, in particular aspects, contemplates introduction of RNA with partial or fully double-stranded character into the cell or into the extracellular environment.

According to the present invention, inhibition is specific to the particular validated KSHV-induced cellular gene expression product in that a nucleotide sequence from a portion of the validated sequence is chosen to produce inhibitory RNA. This process is effective in producing inhibition (partial or complete), and is validated gene-specific. In particular embodiments, the target cell containing the validate gene may be a human cell subject to infection by KSHV (or cell-lines derived therefrom). Methods of preparing and using siRNA are generally disclosed in U.S. Pat. No. 6,506,559, incorporated herein by reference (see also reviews by Milhavet et al., Pharmacological Reviews 55:629-648, 2003; and Gitlin et al., J. Virol. 77:7159-7165, 2003; incorporated herein by reference).

The siRNA may comprise one or more strands of polymerized ribonucleotide, and may include modifications to either the phosphate-sugar backbone or the nucleoside. For example, the phosphodiester linkages of natural RNA may be modified to include at least one of a nitrogen or sulfur heteroatom. Modifications in RNA structure may be tailored to allow specific genetic inhibition while avoiding a general panic response in some organisms which is generated by dsRNA. Likewise, bases may be modified to block the activity of adenosine deaminase. RNA may be produced enzymatically or by partial/total organic synthesis, any modified ribonucleotide can be introduced by in vitro enzymatic or organic synthesis.

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses of double-stranded material may yield more effective inhibition. Inhibition is sequence-specific in that nucleotide sequences corresponding to the duplex region of the RNA are targeted for genetic inhibition. Nucleic acid containing a nucleotide sequence identical to a portion of the validated gene sequence is preferred for inhibition. RNA sequences with insertions, deletions, and single point mutations relative to the target sequence have also been found to be effective for inhibition. Sequence identity may be optimized by alignment algorithms known in the art and calculating the percent difference between the nucleotide sequences. Alternatively, the duplex region of the RNA may be defined functionally as a nucleotide sequence that is capable of hybridizing with a portion of the target gene transcript.

RNA may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region may be used to transcribe the RNA strand (or strands).

For siRNA (RNAi), the RNA may be directly introduced into the cell (i.e., intracellularly); or introduced extracellularly into a cavity, interstitial space, into the circulation of an organism, introduced orally, or may be introduced by bathing an organism in a solution containing RNA. Methods for oral introduction include direct mixing of RNA with food of the organism, as well as engineered approaches in which a species that is used as food is engineered to express a RNA, then fed to the organism to be affected. Physical methods of introducing nucleic acids include injection directly into the cell or extracellular injection into the organism of an RNA solution.

Inhibition of gene expression refers to the absence (or observable decrease) in the level of protein and/or mRNA product from a validated gene target. Specificity refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, fluorescence activated cell analysis (FACS), and KSHV viral infection and/or replication, inhibition of KSHV-induced proliferation, or inhibition of KSHV induced cellular phenotype, as described herein. For RNA-mediated inhibition in a cell line or whole organism, gene expression is conveniently assayed by use of a reporter or drug resistance gene whose protein product is easily assayed. Many such reporter genes are known in the art.

The phosphodiester linkages of natural RNA may be modified to include at least one of a nitrogen or sulfur heteroatom. Modifications in RNA structure may be tailored to allow specific genetic inhibition while avoiding a general panic response in some organisms which is generated by dsRNA. Likewise, bases may be modified to block the activity of adenosine deaminase. RNA may be produced enzymatically or by partial/total organic synthesis, any modified ribonucleotide can be introduced by in vitro enzymatic or organic synthesis.

RNA containing a nucleotide sequences identical to a portion of a particular validated gene sequence are preferred for inhibition. RNA sequences with insertions, deletions, and single point mutations relative to the target sequence may be effective for inhibition. Sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of particular validated gene sequence is preferred. Alternatively, the duplex region of the RNA may be defined functionally as a nucleotide sequence that is capable of hybridizing with a portion of the particular validated gene transcript (e.g., 400 mM NaCl, 40 mM PIPES pH 6.4, 1 mM EDTA, 50° C. or 70° C. hybridization for 12-16 hours; followed by washing). The length of the identical nucleotide sequences may be at least 20, 25, 50, 100, 200, 300 or 400 bases. Preferably, wherein the siRNA agent specific for a validated KSHV-induced cellular gene sequence comprises a nucleic acid sequence of, e.g., at least 9, at least 15, at least 18, or at least 20 contiguous bases in length that is complementary to, or hybridizes under moderately stringent or stringent conditions to a sequence selected from the group consisting of SEQ ID NOS: 1, 3, 5, 7, 9, 11, 13, 25, 27, 29, and sequences complementary thereto.

A 100% sequence identity between the RNA and a particular validated gene sequence is not required to practice the present invention. Thus the methods have the advantage of being able to tolerate sequence variations that might be expected due to genetic mutation, strain polymorphism, or evolutionary divergence.

Particular validated gene sequence siRNA may be synthesized by art-recognized methods either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand (or strands). Inhibition may be targeted by specific transcription in an organ, tissue, or cell type; stimulation of an environmental condition (e.g., infection, stress, temperature, chemical inducers); and/or engineering transcription at a developmental stage or age. The RNA strands may or may not be polyadenylated; the RNA strands may or may not be capable of being translated into a polypeptide by a cell's translational apparatus.

RNA may be chemically or enzymatically synthesized by manual or automated reactions. The RNA may be synthesized by a cellular RNA polymerase or a bacteriophage RNA polymerase (e.g., T3, T7, SP6). The use and production of an expression construct are known in the art (e.g., WO 97/32016; U.S. Pat. Nos. 5,593,874, 5,698,425, 5,712,135, 5,789,214, and 5,804,693; and the references cited therein). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. The RNA may be dried for storage or dissolved in an aqueous solution. The solution may contain buffers or salts to promote annealing, and/or stabilization of the duplex strands.

siRNA may be directly introduced into the cell (i.e., intracellularly); or introduced extracellularly into a cavity, interstitial space, into the circulation of an organism, introduced orally, or may be introduced by bathing an organism in a solution containing the RNA. Methods for oral introduction include direct mixing of the RNA with food of the organism, as well as engineered approaches in which a species that is used as food is engineered to express the RNA, then fed to the organism to be affected. For example, the RNA may be sprayed onto a plant or a plant may be genetically engineered to express the RNA in an amount sufficient to kill some or all of a pathogen known to infect the plant. Physical methods of introducing nucleic acids, for example, injection directly into the cell or extracellular injection into the organism, may also be used. Vascular or extravascular circulation, the blood or lymph system, and the cerebrospinal fluid are sites where the RNA may be introduced. A transgenic organism that expresses RNA from a recombinant construct may be produced by introducing the construct into a zygote, an embryonic stem cell, or another multipotent cell derived from the appropriate organism.

Physical methods of introducing nucleic acids include injection of a solution containing the RNA, bombardment by particles covered by the RNA, soaking the cell or organism in a solution of the RNA, or electroporation of cell membranes in the presence of the RNA. A viral construct packaged into a viral particle would accomplish both efficient introduction of an expression construct into the cell and transcription of RNA encoded by the expression construct. Other methods known in the art for introducing nucleic acids to cells may be used, such as lipid-mediated carrier transport, chemical-mediated transport, such as calcium phosphate, and the like. Thus the RNA may be introduced along with components that perform one or more of the following activities: enhance RNA uptake by the cell, promote annealing of the duplex strands, stabilize the annealed strands, or other-wise increase inhibition of the target gene.

The siRNA may be used alone or as a component of a kit having at least one of the reagents necessary to carry out the in vitro or in vivo introduction of RNA to test samples or subjects. Preferred components are the dsRNA and a vehicle that promotes introduction of the dsRNA. Such a kit may also include instructions to allow a user of the kit to practice the invention.

Suitable injection mixes are constructed so animals receive an average of 0.5×10⁶ to 1.0×10⁶ molecules of RNA. For comparisons of sense, antisense, and dsRNA activities, injections are compared with equal masses of RNA (i.e., dsRNA at half the molar concentration of the single strands). Numbers of molecules injected per adult are given as rough approximations based on concentration of RNA in the injected material (estimated from ethidium bromide staining) and injection volume (estimated from visible displacement at the site of injection). A variability of several-fold in injection volume between individual animals is possible.

Proteins and Polypeptides

In addition to the antisense molecules and ribozymes disclosed herein, inventive modulators of KSHV-induced gene expression also include proteins or polypeptides that are effective in either reducing validated KSHV-induced cellular gene expression or in decreasing one or more of the respective biological activities encoded thereby. A variety of art-recognized methods are used by the skilled artisan, through routine experimentation, to rapidly identify such modulators of KSHV-induced gene expression. The present invention is not limited by the following exemplary methodologies.

Inhibitors of KSHV-induced biological activities encompass those proteins and/or polypeptides that interfere with said biological activities. Such interference may occur through direct interaction with active domains of the proteins of validated gene targets, or indirectly through non- or un-competitive inhibition such as via binding to an allosteric site. Accordingly, available methods for identifying proteins and/or polypeptides that bind to proteins of validated gene targets may be employed to identify lead compounds that may, through the methodology disclosed herein, be characterized for their inhibitory activity.

Methods for detecting and analyzing protein-protein interactions are described in the art, and are thus available to skilled artisans (reviewed in Phizicky, E. M. et al., Microbiological Reviews (1995) 59:94-123 incorporated herein by reference. Such methods include, but are not limited to physical methods such as, e.g., protein affinity chromatography, affinity blotting, immunoprecipitation and cross-linking as well as library-based methods such as, e.g., protein probing, phage display and two-hybrid screening. Other methods that may be employed to identify protein-protein interactions include genetic methods such as use of extragenic suppressors, synthetic lethal effects and unlinked noncomplementation. Exemplary methods are described in further detail below.

Inventive inhibitors of proteins of validated gene targets (validated proteins) may be identified through biological screening assays that rely on the direct interaction between the a validated protein (e.g., SEQ ID NOS:2, 4, 6, 8, 10, 12, 14, 26, 28 and 30) and a panel or library of potential inhibitor proteins. Biological screening methodologies, including the various “n-hybrid technologies,” are described in, for example, Vidal, M. et al., Nucl. Acids Res. (1999) 27(4):919-929; Frederickson, R. M., Curr. Opin. Biotechnol. (1998) 9(1):90-6; Brachmann, R. K. et al., Curr. Opin. Biotechnol. (1997) 8(5):561-568; and White, M. A., Proc. Natl. Acad. Sci. U.S.A. (1996) 93:10001-10003 each of which is incorporated herein by reference.

The two-hybrid screening methodology may be employed to search new or existing target cDNA libraries for inhibitory proteins. The two-hybrid system is a genetic method that detects protein-protein interactions by virtue of increases in transcription of reporter genes. The system relies on the fact that site-specific transcriptional activators have a DNA-binding domain and a transcriptional activation domain. The DNA-binding domain targets the activation domain to the specific genes to be expressed. Because of the modular nature of transcriptional activators, the DNA-binding domain may be severed from the otherwise covalently linked transcriptional activation domain without loss of activity of either domain. Furthermore, these two domains may be brought into juxtaposition by protein-protein contacts between two proteins unrelated to the transcriptional machinery. Thus, two hybrids are constructed to create a functional system. The first hybrid, i.e., the bait, consists of a transcriptional activator DNA-binding domain fused to a protein of interest (e.g., SEQ ID NOS:2, 4, 6, 8, 10, 12, 14, 26, 28 and 30, or fragments thereof). The second hybrid, the target, is created by the fusion of a transcriptional activation domain with a library of proteins or polypeptides. Interaction between the bait protein and a member of the target library results in the juxtaposition of the DNA-binding domain and the transcriptional activation domain and the consequent up-regulation of reporter gene expression.

A variety of two-hybrid based systems are available to the skilled artisan that most commonly employ either the yeast Gal4 or E. coli LexA DNA-binding domain (BD) and the yeast Gal4 or herpes simplex virus VP16 transcriptional activation domain. Chien, C.-T. et al., Proc. Natl. Acad. Sci. U.S.A. (1991) 88:9578-9582; Dalton, S. et al., Cell (1992) 68:597-612; Durfee, T. K. et al., Genes Dev. (1993) 7:555-569; Vojtek, A. B. et al., Cell (1993) 74:205-214; and Zervos, A. S. et al., Cell (1993) 72:223-232. Commonly used reporter genes include the E. coli lacZ gene as well as selectable yeast genes such as HIS3 and LEU2. Fields, S. et al., Nature (London) (1989) 340:245-246; Durfee, T. K., supra; and Zervos, A. S., supra. A wide variety of activation domain libraries is readily available in the art such that the screening for interacting proteins may be performed through routine experimentation.

Suitable bait proteins for the identification of inhibitors of validated proteins are designed based on the validated sequences presented herein as SEQ ID NO:2, 4, 6, 8, 10, 12, 14, 26, 28 and 30. Such bait proteins include either the full-length validated protein, or fragments thereof.

Plasmid vectors, such as, e.g., pBTM116 and pAS2-1, for preparing validated protein bait constructs and target libraries are readily available to the artisan and may be obtained from such commercial sources as, e.g., Clontech (Palo Alto, Calif.), Invitrogen (Carlsbad, Calif.) and Stratagene (La Jolla, Calif.). These plasmid vectors permit the in-frame fusion of cDNAs with the DNA-binding domains as LexA or Gal4BD, respectively.

Validated protein inhibitors of the present invention may alternatively be identified through one of the physical or biochemical methods available in the art for detecting protein-protein interactions.

For example, affinity chromatography may be used to identify potential inhibitors of validated proteins, by virtue of specific retention of such potential inhibitors to validated proteins, or to fragments thereof covalently or non-covalently coupled to a solid matrix such as, e.g., Sepharose beads. The preparation of protein affinity columns is described in, for example, Beeckmans, S. et al., Eur J. Biochem. (1981) 117:527-535 and Formosa, T. et al., Methods Enzymol. (1991) 208:24-45. Cell lysates containing the full complement of cellular proteins may be passed through a validated protein affinity column. Proteins having a high affinity for the validated protein will be specifically retained under low-salt conditions while the majority of cellular proteins will pass through the column. Such high affinity proteins may be eluted from the immobilized validated protein, or fragment thereof under conditions of high-salt, with chaotropic solvents or with sodium dodecyl sulfate (SDS). In some embodiments, it may be preferred to radiolabel the cells prior to preparing the lysate as an aid in identifying the validated protein-specific binding proteins. Methods for radiolabeling mammalian cells are well known in the art and are provided, e.g., in Sopta, M. et al., J. Biol. Chem. (1985) 260:10353-10360.

Suitable validated proteins for affinity chromatography may be fused to a protein or polypeptide to permit rapid purification on an appropriate affinity resin. For example, a validated protein cDNA may be fused to the coding region for glutathione S-transferase (GST) which facilitates the adsorption of fusion proteins to glutathione-agarose columns. Smith et al., Gene (1988) 67:3140. A Iternatively, fusion proteins may include protein A, which can be purified on columns bearing immunoglobulin G; oligohistidine-containing peptides, which can be purified on columns bearing Ni²⁺; the maltose-binding protein, which can be purified on resins containing amylose; and dihydrofolate reductase, which can be purified on methotrexate columns. One such tag suitable for the preparation of validate protein fusion proteins is the epitope for the influenza virus hemagglutinin (HA) against which monoclonal antibodies are readily available and from which antibodies an affinity column may be prepared.

Proteins that are specifically retained on a validated protein affinity column may be identified after subjecting to SDS polyacrylamide gel electrophoresis (SDS-PAGE). Thus, where cells are radiolabeled prior to the preparation of cell lysates and passage through the validated protein affinity column, proteins having high affinity for the particular validate protein may be detected by autoradiography. The identity of particular validated protein-specific binding proteins may be determined by protein sequencing techniques that are readily available to the skilled artisan, such as those described by Mathews, C. K. et al., Biochemistry, The Benjamin/Cummings Publishing Company, Inc. pp. 166-170 (1990).

Antibodies or Antibody Fragments

Inhibitors of KSHV-induced gene expression of the present invention include antibodies and/or antibody fragments that are effective in reducing KSHV-induced gene expression and/or reducing the biological activity encoded thereby. Suitable antibodies may be monoclonal, polyclonal or humanized monoclonal antibodies. Antibodies may be derived by conventional hybridoma based methodology, from antisera isolated from validated protein inoculated animals or through recombinant DNA technology. Alternatively, inventive antibodies or antibody fragments may be identified in vitro by use of one or more of the readily available phage display libraries. Exemplary methods are disclosed herein.

In one embodiment of the present invention, validated protein inhibitors are monoclonal antibodies that may be produced as follows. Validated proteins (SEQ ID NOS:2, 4, 6, 8, 10, 12, 14, 26, 28 and 30) may be produced, for example, by expression of the respective cDNAs (SEQ ID NOS:1, 3, 5, 7, 9, 11, 13, 25, 27 and 29, respectively) in a baculovirus based system. By this method, validated protein cDNAs (SEQ ID NOS:1, 3, 5, 7, 9, 11, 13, 25, 27 and 29) or epitope-bearing fragments thereof are ligated into a suitable plasmid vector that is subsequently used to transfect Sf9 cells to facilitate protein production. In addition, it may be advantageous to incorporate an epitope tag or other moiety to facilitate affinity purification of the validated protein. Clones of Sf9 cells expressing a particular validated protein are identified, e.g., by enzyme-linked immunosorbant assay (ELISA), lysates a re prepared and the validated protein purified by affinity chromatography. The purified validated protein is, for example, injected intraperitoneally, into BALB/c mice to induce antibody production. It may be advantageous to add an adjuvant, such as Freund's adjuvant, to increase the resulting immune response.

Serum is tested for the production of specific antibodies, and spleen cells from animals having a positive specific antibody titer are used for cell fusions with myeloma cells to generate hybridoma clones. Supernatants derived from hybridoma clones are tested for the presence of monoclonal antibodies having specificity against a particular validated protein (e.g., SEQ ID NO:2, 4, 6, 8, 10, 12, 14, 26, 28 and 30, or fragments thereof). For a general description of monoclonal antibody methodology, See, e.g., Harlow and Lane, Antibodies: A Laboratory Manual, Cold Spring Harbor Laboratory (1988).

In addition to the baculovirus expression system, other suitable bacterial or yeast expression systems may be employed for the expression of a particular validated protein or polypeptides thereof. As with the baculovirus system, it may be advantageous to utilize one of the commercially available affinity tags to facilitate purification prior to inoculation of the animals. Thus, the a validated protein cDNA or fragment thereof may be isolated by, e.g., agarose gel purification and ligated in frame with a suitable tag protein such as 6-His, glutathione-S-transferase (GST) or other such readily available affinity tag. See, e.g., Molecular Biotechnology: Principles and Applications of Recombinant DNA, ASM Press pp. 160-161 (ed. Glick, B. R. and Pasternak, J. J. 1998).

In other embodiments of the present invention, inhibitors of validated proteins are humanized anti-validated protein monoclonal antibodies. The phrase “humanized antibody” refers to an antibody derived from a non-human antibody—typically a mouse monoclonal antibody. Alternatively, a humanized antibody may be derived from a chimeric antibody that retains or substantially retains the antigen-binding properties of the parental, non-human, antibody but which exhibits diminished immunogenicity as compared to the parental antibody when administered to humans. The phrase “chimeric antibody,” as used herein, refers to an antibody containing sequence derived from two different antibodies (see, e.g., U.S. Pat. No. 4,816,567) which typically originate from different species. Most typically, chimeric antibodies comprise human and murine antibody fragments, generally human constant and mouse variable regions.

Because humanized antibodies are far less immunogenic in humans than the parental mouse monoclonal antibodies, they can be used for the treatment of humans with far less risk of anaphylaxis. Thus, these antibodies may be preferred in therapeutic applications that involve in vivo administration to a human such as, e.g., use as radiation sensitizers for the treatment of neoplastic disease or use in methods to reduce the side effects of, e.g., cancer therapy.

Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. These methods are disclosed in, e.g., Jones et al., Nature (1986) 321:522-525; Morrison et al., Proc. Natl. Acad. Sci., USA., (1984) 81:6851-6855; Morrison and Oi, Adv. Immunol. (1988) 44:65-92; Verhoeyer et al., Science(1988) 239:1534-1536; Padlan, Molec. Immunol. (1991) 28:489-498; Padlan, Molec. Immunol. (1994) 31(3):169-217; and Kettleborough, C. A. et al., Protein Eng. (1991) 4:773-83 each of which is incorporated herein by reference.

The phrase “complementarity determining region” refers to amino acid sequences which together define the binding affinity and specificity of the natural Fv region of a native immunoglobulin binding site. See, e.g., Chothia et al., J. Mol. Biol. (1987) 196:901-917; Kabat et al., U.S. Dept. of Health and Human Services NIH Publication No. 91-3242 (1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu.

One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region which disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g., via Ashwell receptors (see, e.g., U.S. Pat. Nos. 5,530,101 and 5,585,089, both incorporated herein by reference.

Humanized antibodies to a particular validated protein can also be produced using transgenic animals that are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions, and wherein the endogenous immunoglobulin-encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy claims, and express an exogenous immunoglobulin locus comprising one or more xenogeneic constant regions.

Using a transgenic animal described above, an immune response can be produced to a selected antigenic molecule (e.g., validated protein or fragment thereof), and antibody-producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735. This publication discloses monoclonal antibodies against a variety of antigenic molecules including IL-6, IL-8, TNFα, human CD4, L-selectin, gp39, and tetanus toxin. The monoclonal antibodies can be tested for the ability to inhibit or neutralize the biological activity or physiological effect of the corresponding protein. WO 96/33735 discloses that monoclonal antibodies against IL-8, derived from immune cells of transgenic mice immunized with IL-8, blocked IL-8-induced functions of neutrophils. Human monoclonal antibodies with specificity for the antigen used to immunize transgenic animals are also disclosed in WO 96/34096.

For purposes of the present invention, validated polypeptides and variants thereof a re used to immunize a transgenic animal as described above. Monoclonal antibodies are made using methods known in the art, and the specificity of the antibodies is tested using isolated validated polypeptides. The suitability of the antibodies for clinical use is tested by, for example, exposing KSHV-infected DMVEC cells to the antibodies and measuring cell growth and/or phenotypic changes. According to the invention, inhibition of KSHV-induced gene sequence expression using antisense oligonucleotides specific for validated KSHV-induced polynucleotides causes an inhibition of anchorage-independent growth of KSHV-infected DMVEC cells. The antisense oligonucleotides also inhibited spindle cell formation of KSHV-infected DMVEC cells (or caused reversion of the spindle cell phenotype). Human monoclonal antibodies specific for a particular validated protein, or for a variant or fragment thereof can be tested for their ability to inhibit proliferation, colony growth, or any other biological parameter (e.g., spindle cell formation) indicative of control of tumor growth, migration, or metastasis, particularly tumor cells of epithelial or endothelial origin. Such antibodies would be suitable for pre-clinical and clinical trials as pharmaceutical agents for preventing or controlling growth of cancer cells, including KSHV-related cancer cells.

It will be appreciated that alternative validated protein inhibitor antibodies may be readily obtained by other methods commonly known in the art. One exemplary methodology for identifying antibodies having a high specificity for a particular validated protein is the phage display technology.

Phage display libraries for the production of high-affinity antibodies are described in, for example, Hoogenboom, H. R. et al., Immunotechnology (1998) 4(1):1-20; Hoogenboom, H. R., Trends Biotechnol. (1997) 15:62-70 and McGuinness, B. et al., Nature Bio. Technol. (1996) 14:1149-1154 each of which is incorporated herein by reference. Among the advantages of the phage display technology is the ability to isolate antibodies of human origin that cannot otherwise be easily isolated by conventional hybridoma technology. Furthermore, phage display antibodies may be isolated in vitro without relying on an animal's immune system.

Antibody phage display libraries may be accomplished, for example, by the method of McCafferty et al., Nature (1990) 348:552-554 which is incorporated herein by reference. In short, the coding sequence of the antibody variable region is fused to the amino terminus of a phage minor coat protein (pIII). Expression of the antibody variable region-pIII fusion construct results in the antibody's “display” on the phage surface with the corresponding genetic material encompassed within the phage particle.

A validated protein, or fragment thereof suitable for screening a phage library may be obtained by, for example, expression in baculovirus Sf9 cells as described, supra. Alternatively, the validated protein coding region may be PCR amplified using primers specific to the desired region of the validated protein. As discussed above, the validated protein may be expressed in E. coli or yeast as a fusion with one of the commercially available affinity tags.

The resulting fusion protein may then be adsorbed to a solid matrix, e.g., a tissue culture plate or bead. Phage expressing antibodies having the desired anti-validated protein binding properties may subsequently be isolated by successive panning, in the case of a solid matrix, or by affinity adsorption to a validated protein antigen column. Phage having the desired validated protein inhibitory activities may be reintroduced into bacteria by infection and propagated by standard methods known to those skilled in the art See Hoogenboom, H. R., Trends Biotechnol., supra for a review of methods for screening for positive antibody-pIII phage.

Small Molecules and High-throughput Screening (HTS) Assays

As discussed herein, particular embodiments of the present invention provide screening assays for identification of compounds useful to modulate KSHV infection, comprising: contacting KSHV-infected cells with a test agent; measuring, using a suitable assay, expression of at least one validated KSHV-induced cellular gene sequence; and determining whether the test agent inhibits said validated gene expression relative to control cells not contacted with the test agent, whereby agents that inhibit said validated gene expression are identified as compounds useful to modulate KSHV infection.

Preferably, the at least one validated KSHV-induced cellular gene sequence is selected from the cDNA and protein sequence group consisting of RDC-1, IGFBP2, FLJ14103, KIAA0367, Neuritin, INSR, KIT, LOX, NOV and ANGPTL2, and combinations thereof (i.e., consisting of SEQ ID NOS:1-14 and SEQ ID NOS:25-30). Preferably, expression of at least one validated KSHV-induced cellular gene sequence is expression of at least one of mRNA, or expression of the protein encoded thereby. Preferably, agents that inhibit said validated gene expression are further tested for the ability to modulate KSHV-mediated effects on cellular proliferation and/or phenotype.

The present invention also provides small molecule modulators that may be readily identified through routine application of high-throughput screening (HTS) methodologies. Reviewed by Persidis, A., Nature Biotechnology (1998) 16:488-489. H TS methods generally permit the rapid screening of test compounds, such as small molecules, for therapeutic potential. HTS methodology employs robotic handling of test materials, detection of positive signals and interpretation of data. Such methodologies include, e.g., robotic screening technology using soluble molecules as well as cell-based systems such as the two-hybrid system described in detail above.

A variety of cell line-based HTS methods are available that benefit from their ease of manipulation and clinical relevance of interactions that occur within a cellular context as opposed to in solution. Test compounds are identified via incorporation of radioactivity or through optical assays that rely on absorbance, fluorescence or luminescence as read-outs. See, e.g., Gonzalez, J. E. et al., Curr. Opin. Biotechnol. (1998) 9(6):624-631 incorporated herein by reference.

HTS methodology is employed, e.g., to screen for test compounds that modulate or block one of the biological activities of a validated protein (i.e., a protein encoded by validated KSHV-induced cellular gene expression). For example, a validated protein may be immunoprecipitated from cells expressing the protein and applied to wells on an assay plate suitable for robotic screening. Individual test compounds are contacted with the immunoprecipitated protein and the effect of each test compound on an activity of the validated protein is assessed. For example, if the particular validated protein has kinase activity, the effect of a particular test compound on the kinase is assessed by, e.g., incubating the corresponding immunopreciped protein in contact with the particular test compound in the presence of γ-³²P-ATP in a suitable buffer system, and measuring the incorporation of ³²P Both small molecule agonists and antagonists of particular validated proteins (SEQ ID NOS:2, 4, 6, 8 10, 12, 14, 26, 28 and 30) are encompassed within the scope of the present invention.

Preferably, KSHV-infected DMVEC are used in inventive screening assays for therapeutic compounds.

Gleevec™, for example, as described by Moses et al., J. Virol. 76:8383-8399, 2002 (see also WO0210339A2), is a representative example of a small molecule modulator of c-Kit tyrosine kinase activity that modulates KSHV-induced cellular gene expression. STI 571 (Gleevec™) was designed as an ATP-competitive inhibitor of the Ab1 tyrosine kinase, and was later shown to be active against c-Kit (Heinrich et al., Blood 96:925-932m 2000).

The proliferative response of KSHV-infected DMVEC to exogenous SCF is inhibited by STI 571, where cell viability controls show that such growth inhibition is not due to nonspecific cytotoxicity of STI 571 (see Moses et al., supra). The c-Kit-mediated inhibition by STI 571 of KSHV-infected DMVEC proliferation identifies STI 571 as a therapeutic modulator of KSHV-induced gene expression.

Additionally, as discussed herein, KSHV-infected DMVEC develop a spindle phenotype and exhibit transformed characteristics including disorganized growth, focus formation and anchorage-independent growth in semisolid agar. Following treatment of KSHV-infected DMVEC with STI 571 to inhibit endogenous c-Kit tyrosine kinase activity, focus formation is inhibited and an organized monolayer with distinct cell margins is reestablished (Id): Moreover, removal of STI 571 leads to regeneration of the transformed phenotype, even after exposure of cells to a 10 μM dose (Id). Uninfected DMVEC exhibit normal growth with an organized cobblestone phenotype when maintained at confluency, and exposure to STI 571 has effect on cell morphology or viability.

The ability to reverse KSHV-induced morphological transformation through specific inhibition of c-Kit activity further demonstrates a critical role for c-Kit signaling in KSHV-induced transformation of endothelial cells and further supports a role for upregulation of c-Kit as a factor in KS tumorigenesis.

Likewise, modulators of the present novel validated KSHV-induced cellular gene expression are identified by the inventive screening assays.

Methods for Assessing the Efficacy of Modulators of either KSHV-induced Gene Expression or of Biological Activity Encoded thereby

Inventive modulators or compounds, whether antisense molecules or ribozymes, proteins and/or peptides, antibodies and/or antibody fragments or small molecules, that are identified either by one of the methods described herein or via techniques that are otherwise available in the art, may be further characterized in a variety of in vitro, ex vivo and in vivo animal model assay systems for their ability to modulate or inhibit KSHV-induced gene expression or biological activity. As discussed in further detail in the Examples provided below, particular inventive modulators of KSHV-induced gene expression are antisense inhibitors effective in reducing KSHV-induced cellular gene expression levels. Thus, the present invention describes, teaches and supports methods that permit the skilled artisan to assess the effect of candidate modulators and inhibitors.

For example, candidate modulators or inhibitors of KSHV-induced gene expression are tested by administration of such candidate modulators to cells that express KSHV-induced genes and gene products, such as KSHV-infected DMVEC in the inventive soft agar system. KSHV-infected mammalian cells may also be engineered to express a given KSHV-induced gene or recombinant reporter molecule introduced into such cells with a recombinant KSHV-inducible gene plasmid construct.

Effective modulators of KSHV-induced gene expression that are inhibitors will be effective in reducing the levels of KSHV-induced gene mRNA as determined, e.g., by Northern blot or RT-PCR analysis. For a general description of these procedures, see, e.g., Sambrook et al., Molecular Cloning: A Laboratory Manual Cold Spring Harbor Press (1989) and Molecular Biotechnology: Principles and Applications of Recombinant DNA, ASM Press (ed. Glick, B. R. and Pastemak, J. J. 1998) incorporated herein by reference. The effectiveness of a given candidate antisense molecule may be assessed by comparison with a control ‘antisense’ molecule (e.g., a reverse complement control oligonucleotide, corresponding in orientation and size to the coding sequence complementary to the candidate antisense molecule) known to have no substantial effect on KSHV-induced gene expression when administered to a mammalian cell. Exemplary control molecules include KSHV-inducible gene sequence-specific reverse complement oligonucleotides corresponding to one of the inventive antisense molecules described herein above, or to preferred representative thereof (e.g., reverse complement control oligonucleotides for SEQ ID NOS:15-21 and SEQ ID NOS:31-33).

In alternate embodiments of the present invention, the effect of modulators and inhibitors of KSHV-induced gene expression on the rate of DNA synthesis after challenge with a radiation or chemotherapeutic agent may be assessed by, e.g., the method of Young and Painter. Hum. Genet. (1989) 82:113-117. Briefly, culture cells may be incubated in the presence of ¹⁴C-thymidine prior to exposure to, e.g., X-rays. Immediately after irradiation, cells are incubated for a short period prior to addition of ³H-thymidine. Cells are washed, treated with perchloric acid and filtered (Whatman GF/C). The filters are rinsed with perchloric acid, 70% alcohol and then 100% ethanol; radioactivity is measured and the resulting ³H/¹⁴C ratios used to determine the rates of DNA synthesis.

Animal model systems. Modulators or inhibitors of KSHV-induced gene expression effective in modulating or reducing KSHV-induced cellular gene expression by one or more of the methods discussed above are further characterized in vivo for efficacy one or more available art-recognized animal model systems. Various animal model systems for study of cancer and genetic instability associated genes are disclosed in, for example, Donehower, L. A. Cancer Surveys (1997) 29:329-352 incorporated herein by reference. In particular, various art-recognized animal model systems for testing PMO antisense oligonucleotide agents, including xenograft murine models are discussed Devi, Current Opinion in Molecular Therapeutics, 4:138-148, 2002, incorporated by reference herein.

Pharmaceutical Compositions

The antisense oligonucleotides and ribozymes of the present invention are synthesized by any method known in the art for ribonucleic or deoxyribonucleic nucleotides. For example, the oligonucleotides are prepared using solid-phase synthesis such as in an Applied Biosystems 380B DNA synthesizer. Final purity of the oligonucleotides is determined as is known in the art.

The antisense oligonucleotides identified using the methods of the invention modulate cancer cell proliferation, including anchorage-independent proliferation, and also modulate KSHV-mediated phenotypic changes, including spindle formation.

Therefore, pharmaceutical compositions and methods are provided for interfering with cell proliferation, preferably cancer or tumor cell proliferation, comprising contacting tissues or cells with one or more of antisense oligonucleotides identified using the methods of the invention. Preferably, an antisense oligonucleotide having one of SEQ ID NOS:15-21 and SEQ ID NOS:31-33 is administered. Preferably, the antisense oligonucleotide is a PMO antisense oligomer (PMO).

The methods and compositions may also be used to treat other KSHV-associated proliferative disorders including sarcomas, and KSHV-related neoangiogenesis (neovascularization).

The invention provides pharmaceutical compositions of antisense oligonucleotides and ribozymes complementary to validated KSHV-induced cellular gene mRNA gene sequences, corresponding to SEQ ID NOS:1, 3, 5, 7, 9, 11, 13, 25, 27 and 29 as active ingredients for therapeutic application. These compositions can also be used in the methods of the present invention. Where required the compounds are nuclease resistant. In general the pharmaceutical composition for modulating KSHV-mediated cellular proliferation or phenotype in a mammal includes an effective amount of at least one antisense oligonucleotide as described above needed for the practice of the invention, or a fragment thereof shown to have the same effect, and a pharmaceutically physiologically acceptable carrier or diluent.

Particular embodiments provide a method for reducing KSHV-mediated cellular proliferation and/or phenotypic differentiation in a subject comprising administering an amount of an antisense oligonucleotide of the invention effective to reduce said KSHV-mediated cellular proliferation and/or phenotypic differentiation. Preferably the antisense oligomer is based on one of SEQ ID NOS:1, 3, 5, 7, 9, 11, 13, 25, 27 and 29. More preferably the antisense oligonucleotide is one of SEQ ID NOS:15-21 and SEQ ID NOS:31-33.

The pharmaceutical composition for inhibiting tumorigenicity of neoplastic cells in a mammal consists of an effective amount of at least one active ingredient selected from antisense oligonucleotides complementary to the KSHV-induced cellular gene mRNA, including the entire KSHV-induced gene mRNA or having shorter sequences as set forth in SEQ ID NOS:15-21 and SEQ ID NOS:31-33, and a pharmaceutically acceptable carrier or diluent. Combinations of the active ingredients are contemplated and encompassed within the scope of the invention.

The compositions can be administered orally, subcutaneously or parenterally including intravenous, intraarterial, intramuscular, intraperitoneally, and intranasal administration as well as intrathecal and infusion techniques as required by the malignant cells being treated. For delivery within the CNS intrathecal delivery can be used with for example an Ommaya reservoir or other methods known in the art. The pharmaceutically acceptable carriers, diluents, adjuvants and vehicles as well as implant carriers generally refer to inert, non-toxic solid or liquid fillers, diluents or encapsulating material not reacting with the active ingredients of the invention. Cationic lipids may also be included in the composition to facilitate oligonucleotide uptake. Implants of the compounds are also useful. In general, the pharmaceutical compositions are sterile.

In the method of the present invention, KSHV-related proliferating cells, including neoplastic cells are contacted with a growth-inhibiting amount of the bioactive antisense oligonucleotide for the KSHV-induced cellular gene mRNA or a fragment thereof shown to have substantially the same effect. In an embodiment, the mammal to be treated is human but other mammalian species can be treated in veterinary applications.

Bioactivity, relating to a particular oligonucleotide modulator, refers to biological activity in the cell when the oligonucleotide modulator is delivered directly to the cell and/or is expressed by an appropriate promotor and active when delivered to the cell in a vector as described below. Nuclease resistance of particular modulators is provided by any method known in the art that does not substantially interfere with biological activity as described herein.

Significantly, PMO chemistry is not RNase H competent (discussed in Devi, Current Opinion in Molecular Therapeutics, 4:138-148, 2002).

“Contacting the cell” refers to methods of exposing, delivery to, or ‘loading’ of a cell of antisense oligonucleotides whether directly or by viral or non-viral vectors, and where the antisense oligonucleotide is bioactive upon delivery. The method of delivery will be chosen for the particular cancer being treated. Parameters that affect delivery can include the cell type affected and tumor location as is known in the medical art.

The treatment generally has a length proportional to the length of the disease process and drug effectiveness and the patient species being treated. It is noted that humans are treated generally longer than the Examples exemplified herein, which treatment has a length proportional to the length of the disease process and drug effectiveness. The doses may be single doses or multiple doses as determined by the medical practitioners and treatment courses will be repeated as necessary until diminution of the disease is achieved. Optimal dosing schedules may be calculated using measurements of drug accumulation in the body. Practitioners of ordinary skill in the art can readily determine optimum dosages, dosing methodologies, and repetition rates. Optimum dosages may vary depending on the relative potency of the antisense oligonucleotide, and can generally be determined based on values in in vitro and in vivo animal studies and clinical trials. Variations in the embodiments used may also be utilized. The amount must be effective to achieve improvement including but not limited to decreased tumor growth, or tumor size reduction, or to improved survival rate or length or decreased drug resistance or other indicators as are selected as appropriate measures by those skilled in the art.

Although particular inventive antisense oligonucleotides may not completely abolish tumor cell growth, or KSHV-induced proliferation or differentiation in vitro, as exemplified herein, these antisense compounds are nonetheless clinically useful where they inhibit KSHV-related tumor growth enough to allow complementary treatments, such as chemotherapy or radiation therapy, to be effective or more effective. The pharmaceutical compositions of the present invention therefore are administered singly or in combination with other drugs, such as cytotoxic a gents, immunotoxins, alkylating agents, anti-metabolites, antitumor antibiotics and other anti-cancer drugs and treatment modalities that are known in the art.

Cocktails of antisense inhibitors directed against several KSHV-induced gene sequences are contemplated and within the scope of the present invention.

The composition is administered and dosed in accordance with good medical practice taking into account the clinical condition of the individual patient, the site and method of administration, scheduling of administration, and other factors known to medical practitioners. The “effective amount” for growth inhibition is thus determined by such considerations as are known in the art. The pharmaceutical composition may contain more than one embodiment or modulator of the present invention.

The nucleotide sequences of the present invention can be delivered either directly or with viral or non-viral vectors. When delivered directly the sequences are generally rendered nuclease resistant. Alternatively, the sequences can be incorporated into expression cassettes or constructs such that the sequence is expressed in the cell. Generally, the construct contains the proper regulatory sequence or promoter to allow the sequence to be expressed in the targeted cell.

Once the oligonucleotide sequences are ready for delivery, they can be introduced into cells as is known in the art (see, e.g., Devi, Current Opinion in Molecular Therapeutics, 4:138-148, 2002). Transfection, electroporation, fusion, liposomes, colloidal polymeric particles and viral vectors as well as other means known in the art may be used to deliver the oligonucleotide sequences to the cell. The method selected will depend at least on the cells to be treated and the location of the cells and will be known to those skilled in the art. Localization can be achieved by liposomes, having specific markers on the surface for directing the liposome, by having injection directly into the tissue containing the target cells, by having depot associated in spatial proximity with the target cells, specific receptor mediated uptake, viral vectors, or the like.

Administration and clinical dosing of PMO antisense therapeutic agents is discussed, for example, in Devi, supra, and in Arora et al. Journal of Pharmaceutical Sciences, 91:1009-1018, 2001, both incorporated by reference herein.

The present invention provides vectors comprising an expression control sequence operatively linked to the oligonucleotide sequences of the invention. The present invention further provides host cells, selected from suitable eukaryotic and prokaryotic cells, which are transformed with these vectors as necessary. Such transformed cells allow the study of the function and the regulation of malignancy and the treatment therapy of the present invention.

Vectors are known or can be constructed by those skilled in the art and should contain all expression elements necessary to achieve the desired transcription of the sequences. Other beneficial characteristics can also be contained within the vectors such as mechanisms for recovery of the oligonucleotides in a different form. Phagemids are a specific example of such beneficial vectors because they can be used either as plasmids or as bacteriophage vectors. Examples of other vectors include viruses such as bacteriophages, baculoviruses and retroviruses, DNA viruses, liposomes and other recombination vectors. The vectors can also contain elements for use in either prokaryotic or eukaryotic host systems. One of ordinary skill in the art will know which host systems are compatible with a particular vector.

The vectors can be introduced into cells or tissues by any one of a variety of known methods within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al., BioTechniques (1986) 4:504-512 and include, for example, stable or transient transfection, lipofection, electroporation and infection with recombinant viral vectors.

Recombinant methods known in the art can also be used to achieve the antisense inhibition of a validated target nucleic acid. For example, vectors containing antisense nucleic acids can be employed to express an antisense message to reduce the expression of the validated target nucleic acid and therefore its activity.

The present invention also provides a method of evaluating if a compound inhibits transcription or translation of an KSHV-induced cellular gene sequence, and thereby modulates (i.e., reduces) cell proliferation or phenotypic differentiation, comprising transfecting a cell with an expression vector comprising a nucleic acid sequence encoding a KSHV-induced cellular gene sequence, the necessary elements for the transcription or translation of the nucleic acid; administering a test compound; and comparing the level of expression of the KSHV-induced cellular gene sequence with the level obtained with a control in the absence of the test compound. Alternatively, as is shown in the Examples herein, such an expression vector is not required, and test compounds are administered to KSHV-infected cells, such as KSHV-infected DMVEC.

The present invention provides detectably labeled oligonucleotides for imaging KSHV-induced cellular gene sequences (polynucleotides) within a cell. Such oligonucleotides are useful for determining if gene amplification has occurred, for assaying the expression levels in a cell or tissue using, for example, in situ hybridization as is known in the art, and for diagnostic and/or prognostic purposes.

Diagnostic and/or Prognostic Assays for KSHV-Related Cancer

The present invention provides for diagnostic and/or prognostic cancer assays based on differential measurement of validated KSHV-induced gene expression. Preferred validated KSHV-induced gene sequences are represented herein by SEQ ID NOS:1, 3, 5, 7, 9, 11, 13, 25, 27 and 29.

Typically, such assays involve obtaining a tissue sample from a test tissue, performing an assay to measure expression of at least one validated KSHV-induced gene sequence (e.g., mRNA or protein encoded thereby) derived from the tissue sample, relative to a control sample, and making a diagnosis or prognosis based thereon.

In particular embodiments the present inventive oligomers, such as those based on SEQ ID NOS:1, 3, 5, 7, 9, 11, 13, 25, 27 and 29, or preferably SEQ ID NOS:15-21 and SEQ ID NOS:31-33, or arrays thereof, as well as a kit based thereon are useful for the diagnosis and/or prognosis of cancer and/or other KSHV-related cell proliferative disorders.

The present invention moreover relates to a method for manufacturing a diagnostic agent and/or therapeutic agent for the diagnosis and/or therapy of KSHV-related diseases, the diagnostic agent and/or therapeutic agent being characterized in that at least one inventive modulator of KSHV-induced gene expression is used for manufacturing it, possibly together with suitable additives and ancillary agents.

Diagnostic kits are also contemplated, comprising at least one primer and/or probe specific for a validated KSHV-induced cellular gene sequence according to the present invention, possibly together with suitable additives and ancillary agents.

While the present invention has been described with specificity in accordance with certain of its preferred embodiments, the following examples serve only to illustrate the invention and are not intended to limit the invention.

### EXAMPLE 1

### KSHV-Infected DMVECs are a Valid Model System for In Vivo Tumorogenesis

Soft Agar Cell Growth Systems. The soft agar assay system is an art-recognized in vitro cell growth/differentiation system to model in vivo cancer. Particularly, out of a host of exemplary references, see: Tomkowicz, K et al., DNA Cell Biol. 21:151, 2002 (use of soft agar assays system to demonstrate transformation with KSHV kaposin protein); Saucier et al., Oncogene 21:1800, 2002 (use of soft agar assays system to demonstrate transformation with Met RTK protein); and see also Chernicky, CL, Mol. Pathol. 55:102, 2002 (use of inhibition of colony formation in soft agar as validation for siRNS inhibition of a tumor growth factor).

KSHV-infected DMVEC. DMVECs were used as an in vitro model for examining cancerous transformation and viral replication, based, inter alia, on that fact that neoplastic cells in KS tumors are predominantly of vascular origin, whereas KSHV is primarily found in cells of endothelial origin. Specifically, a previously described DMVEC system (Moses et al., J. Virol. 73:6892-6902, 1999) was used for studying infection and transformation by KSHV. Briefly, DMVEC's were immortalized with the E6/E7 genes of human papillomavirus (HPV)-16 prior to infection with KSHV. While transformation with HPV-E6 and HPV-E7 immortalizes DMVEC, they do not develop the KS-typical spindle shape (Staskus, K. A., et al., J. Virol. 71:715-9, 1997) unless infected with KSHV. KSHV was obtained from the supernatant of KSHV-infected B-cell lines (e.g., TPA-stimulated BCBL-1 cells). Infection was verified by DNA PCR for amplification of the KS330 BamH1 fragment of the ORF 26 gene, and RT-PCR for the spliced mRNA from the ORF29 gene. The percentage of latently infected cells was determined by immunofluorescent staining for LANA/ORF73. Lytic induction was evaluated with antibodies against an early lytic protein ORF59/PF-8 and a late lytic glycoprotein ORF K8.1A/B. DMVEC were used for experiments when 90% of cells expressed ORF73. In the absence of chemical induction, 2-5% of infected cells expressed ORF59 with approximately 10% of the ORF59-positive cells expressing K8.1A/B. Lytic replication can be induced, however, using phorbol esters such as phorbol-112-myristate-13 acetate (PMA) providing the ability to look for host genes involved in the lytic cycle as well.

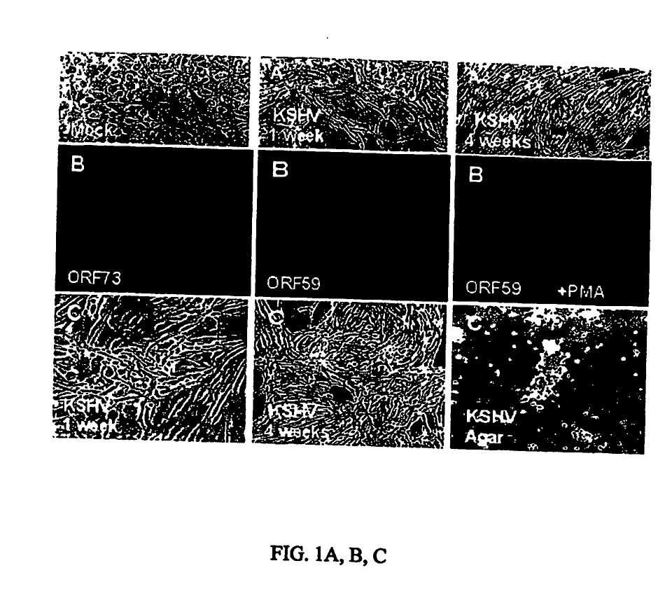

FIGS. 1A, B and C show data from experiments performed to illustrate three hallmarks of the KSHV-DMVEC model system that support its art-recognized utility for mimicking the in vivo system.

First, FIG. 1A shows that immortalized DMVEC cells grow with a characteristic cobblestone morphology in the absence of KSHV infection but change to a spindle cell morphology one (central-panel) to four weeks (rightmost-panel) following infection with KSHV. Specifically, FIG. 1A shows dermal microvascular endothelial cells (DMVECs) that were uninfected (“Mock”) (left-most panel), 1-week post-infection (central panel), or 4-weeks post-infection (right-most panel). The beginning of characteristic spindle cell formation in DMVEC cells was observed 1-week post-infection with KSHV, and substantially progressed through 4 weeks post-infection.

FIG. 1B shows a second feature of the KSHV-DMVEC model system that mimics the in vivo situation; namely, that KSHV enters the lytic replication cycle spontaneously in only approximately 2% of the cells (compare left-most and central panels of FIG. 1B). This ratio, as described above, was visualized by immunofluorescence with antibodies that recognize the products of viral genes expressed during latency (ORF 73, LANA-1) (left-most panel) or viral proteins that are only expressed upon entering the lytic phase (ORF 59) (central panel). Lytic replication can be, and was induced, however, using phorbol esters such as PMA providing the ability to look for host genes involved in the lytic cycle as well (right-most panel). Specifically, FIG. 1B shows fluorescent staining of latent KSHV-infected DMVEC cells (“ORF7,” left-most panel), fluorescent staining of lytic KSHV infected DMVEC cells (“B-ORF59,” central panel), and fluorescent staining of lytic KSHV-infected DMVEC cells enhanced with PMA (“ORF59+PMA,” right-most panel). Phorbol-112-myristate-13 acetate (PMA) was purchased from Calbiochem (San Diego, Calif.).

Third, FIG. 1C shows that while immortalized DMVECs are unable to form foci or grow in soft agar in the absence of KSHV infection, they exhibit hallmarks of transformation following KSHV infection; namely, loss of contact inhibition, and acquisition of anchorage-independent growth. Specifically, FIG. 1C shows the beginning of foci formation in KSHV-infected DMVEC observed at 1-week post infection (“KSHV 1 week,” left-most panel), progression of foci formation observed at 4-weeks post infection (“KSHV 4 weeks,” central panel), and KSHV-infected DMVECs observed growing in soft agar as a result of the acquisition of anchorage-independent growth (“KSHV Agar,” right-most panel).

These phenotype changes, illustrated by the experimental data of FIGS. 1A, B and C, formed the basis for the primary biological assays used herein to validate regulated cellular genes and/or gene products as therapeutic targets.

### EXAMPLE 2

### Nucleic Acid Microarray Technology was Used for Gene Expression Profiling of KSHV-Infected Dermal Microvascular Endothelial Cells (DMVEC) to Identify Cellular Genes Whose Expression is Regulated by KSHV

Nucleic Acid Microarray Data Analysis. Altered expression of cellular genes frequently represents the ultimate cause of tumor formation. In the case of virally-induced tumors, viral genes modulate the host cell gene expression program that is in turn responsible for the transformed phenotype. Cellular genes involved in the transformed phenotype caused by latent infection with KSHV were identified by using DNA microarrays to examine the differential gene expression profiles of primary dermal microvascular endothelial cells (DMVEC) before and after KSHV-infection.

For RNA isolation and fluorescent labeling, two RNA probe samples from DMVEC cells, independently infected with KSHV, and two independent uninfected RNA probe samples were prepared. Briefly, experiments were performed on cells shortly after spread of infection to the majority of cells and development of spindle cells. Specifically, RNA was routinely isolated approximately 4-6 weeks post-infection, after initial infection when >90% of the cells were LANA positive and showed spindle cell phenotype. RNA was isolated from T75 flasks containing approximately 5×10⁶ cells using the RNeasy™ RNA isolation kit (QIAGEN Inc., Valencia, Calif.). After DNase treatment and another round of RNeasy purification, labeled cDNA was prepared as described previously (see Salunga et al., In M. Schena (ed.), DNA microarrays. A practical approach; Oxford Press, Oxford, United Kingdom, 1999; and see Simmen et al., Proc. Natl. Acad. Sci. USA 98:7140-7145, 2001). Briefly, double-stranded cDNA was selectively synthesized from the RNA samples. Biotin-labeled cRNA was produced from the cDNA by in vitro transcription (IVT) using methods well known in the art.

For expression profile screening, the biotin labled cRNA probe preparations were fragmented and hybridized to Affymetrix (Santa Clara, Calif.) U133A and U133B arrays or to U95A arrays (Affymetrix U133A, U133B and U95A GeneChip® arrays). The Human Genome U133 (HG-U133) set, consists of two GeneChip® arrays, and contains almost 45,000 probe sets representing more than 39,000 transcripts derived from approximately 33,000 well-substantiated human genes (Affymetrix technical information). The set design uses sequences selected from GenBank®, dbEST, and RefSeq (Id).

The Affymetrix GeneChip® platform was chosen for these studies as it is the industry leader in terms of array content, platform stability and data quality. Images of the arrays were analyzed using the Affymetrix microarray analysis suite software, MAS. This software package is used for converting images to raw numerical data, and direct comparisons between control and experimental samples. When making such comparisons, MAS provides robust statistical algorithms for determining changes in expression between the two samples, along with p-values and confidence limits on such changes. For each probe set, MAS records whether there was no change, increased expression or decreased expression.

To determine if the number of gene expression changes in common between two or more experiments is significant, we compare the number of genes in such lists to the number expected if the experiments were independent. In the present KSHV experiments, there are approximately 10-fold more gene changes in common between infections than predicted for independent experiments.

Each of the DMVEC infected/uninfected sample comparisons resulted in approximately 480 probe sets with increased expression, with 316 probe sets that showed increased expression in both infections. There were 390 probes sets that showed decreased expression in both, out of approximately 600 probe sets that were down in the individual experiments. Increased or decreased expression was based on ‘calls’ from MAS software which typically corresponds to about a two-fold change. The 706 probes sets identified with significant changes in expression correspond to 580 unique gene sequences.

Representative microarray expression data. TABLE 1 shows expression data obtained according to the present invention for the RDC1, IGFBP2, FLJ14103, KIAA0367, Neuritin, INSR, KIT, IFACTOR, LMO2, MFAP3, LOX, NOV and ANGTPL2 gene sequences using Affymetrix U133 and U95 arrays as indicated. Expression is presented as “fold-increase” in signal for two to four independent infected/mock infected comparisons, as described herein above.

TABLE 1
U133 and U95A microarray expression data for
particular KSHV-induced gene sequences.

| | | | FOLD | FOLD |
|---|---|---|---|---|
| | | | INCREASE; | INCREASE; |
| | | Affymetrix | I1219 × | I0109 × |
| GENE | ARRAY | Probe Set | M1219 | M0109 |
| RDC-1 | UI33A | 212977_at | 34 | 87 |
| | U95A | 34288_at | 37.9 | 36.1 |
| IGFBP2 | UI33A | 202718_at | 2.7 | 1.8 |
| | U95A | 40422_at | 2.3 | 3.5 |
| FLJ14103 | UI33A | 219652_s_at | 30.2 | 44.7 |
| | UI33A | 222911_s_at | 3.8 | 4.7 |
| KIAA0367 | U133A | 212805_at | 2.4 | 2.6 |
| | U133A | 212806_at | 3.2 | 2.6 |
| | U95A | 33442_at | 3.3 | 3.2 |
| Neuritin | n/a | n/a | n/a | n/a |
| INSR | U133A | 213792_s_at | 2.6 | 2.7 |
| | U133B | 227432_s_at | 2.5 | 3.4 |
| | U95A | 1572_s_at | 3.6 | 11.4 |
| KIT | U133A | 205051_s_at | 34 | 20.9 |
| | U95A | 1888_s_at | ˜10.8 | ˜30.1 |
| IFACTOR | UI33A | 203854_at | 21.6 | 39.4 |
| LMO2 | UI33A | 204249_s_at | 2.2 | 2.8 |
| MFAP3 | UI33A | 213123_at | 2.5 | 2.7 |
| | UI33A | 214588_s_at | 10.9 | 4.4 |
| | U95A | 35217_at | 3.4 | 4.5 |
| LOX | U133A | 215446_s_at | 1.62 | 3.48 |
| | U133A | 213640_s_at | 1.07 | 2.3 |
| NOV | U133A | 204298_s_at | 1.32 | 3.48 |
| | U133A | 214321_at | 5.66 | 8 |
| | U133A | 204501_at | 2.83 | 5.28 |
| ANGPTL2 | U133A | 213004_at | 1.52 | 3.03 |
| | U133A | 213001_at | 1.74 | 3.48 |

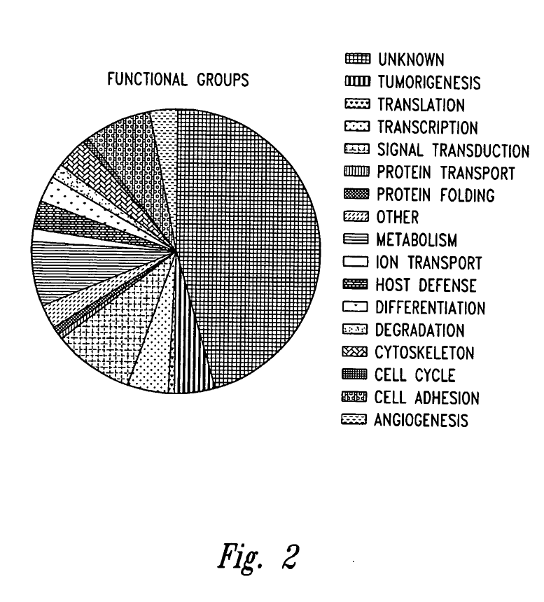

Functional grouping of identified gene sequences. FIG. 2 shows a placement of the genes identified as having statistically significant altered expression in KSHV-infected (latent) DMVEC into functional groups, based on information available in the art.

### EXAMPLE 3

### Target Validation; Genes Necessary for Virally-Induced Morphological Changes in KSHV-Infected DMVEC were Identified Using Antisense PMOs

Antisense Phosphorodiamidate Morpholino Oligomers (PMOs). PMOs (see, e.g., Summerton, et al., Antisense Nucleic Acid Drug Dev. 7:63-70, 1997; and Summerton & Weller, Antisense Nucleic Acid Drug Dev. 7:187-95, 1997) are a class of antisense drugs developed for treating various diseases, including cancer. For example, Arora et al. (J. Pharmaceutical Sciences 91:1009-1018, 2002) demonstrated that oral administration of c-myc-specific and CYP3A2-specific PMOs inhibited c-myc and CYP3A2 gene expression, respectively, in rat liver by an antisense mechanism of action. Likewise, Devi G. R. (Current Opinion in Molecular Therapeutics 4:138-148, 2002) discusses treatment of prostate cancer with various PMO therapeutic agents).

PMOs were designed and used, according to the present invention to silence genes identified as being consistently up-regulated in KSHV-infected DMVEC. PMOs do not activate RNAse H, and inhibit translation by steric hindrance at the ribosome binding site (Ghosh, et al. Methods in Enzymology 313:135-143, 2000). Typically, it is preferable and sufficient to target the region of the start codon to block translation, but, as discussed herein above, other mRNA regions, both coding and non-coding can be effectively targeted according to the present invention.

Antisense Gene Silencing using PMOs. Genes identified as being consistently up-regulated in KSHV-infected DMVEC in the above described nucleic acid microarray/gene expression profiling experiments were further analyzed to identify those necessary for virally-induced cell morphology changes. Silencing of such genes precluded progression into the transformed phenotype when silencing occurred prior to transformation, or induced reversion to the normal state when silencing occurred after induction of the transformed state (see TABLE 2 below).

Therefore, the present invention provides for particular validated cellular gene targets, and for respective therapeutic methods and compositions for blocking virally-induced morphological changes and treating or preventing cancer.

Introduction of antisense PMO into KSHV-infected DMVEC. Antisense PMO molecules, for delivery purposes, are typically converted to a paired duplex together with a partially complementary cDNA oligonucleotide in the weakly basic delivery reagent ethoxylated polyethylenimine (EPEI) (Summerton, supra). The anionic complex binds to the cell surface, is taken up by endocytosis and eventually released into the cytosol. A protocol for optimum uptake of antisense PMO in immortalized DMVEC was developed using a modification of the EPEI method. Briefly, uninfected, immortalized DMVECs were incubated for 3 hours at 37° C. with 0.6 nmol/well FITC-PMO complexed with EPEI according to the manufacturer's instructions (Genetools, LLC, One Summerton Way, Philomath, OR 97370) (e.g., 1.25 n Mol oligomer with 2.5 μl EPEI reagent per 35 mm dish, allowing for sufficient antisense uptake without non-specific EPEI-induced toxicity). The PMOs were labeled with FITC to allow for monitoring of loading efficiency by fluorescence microscopy.

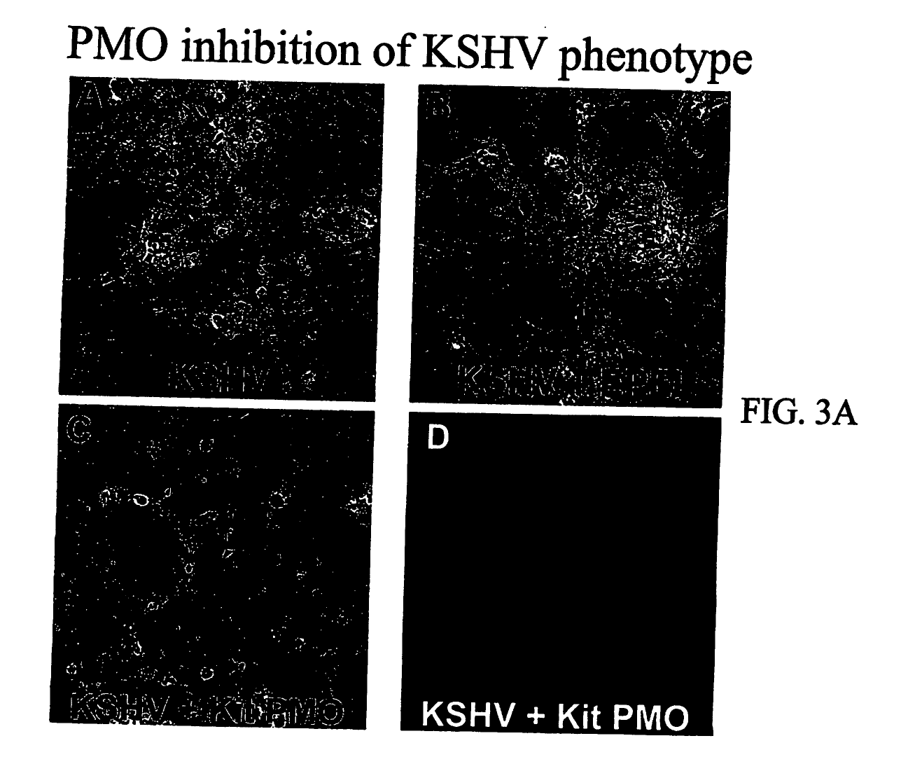

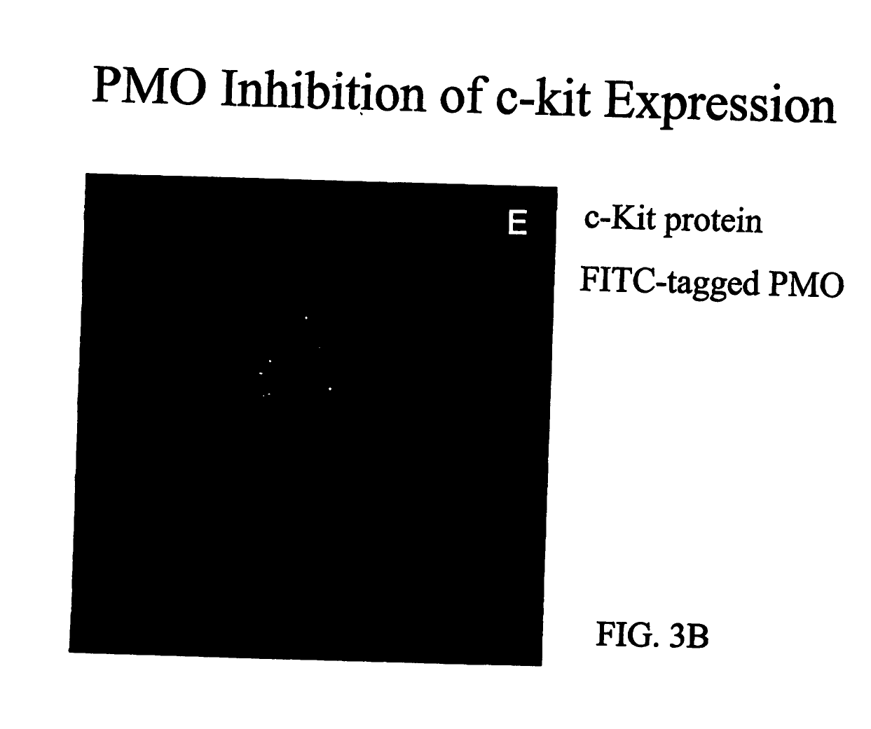

Cellular distribution of introduced FITC-labeled POM antisense molecules. FIG. 3A (lower-right panel “D”) shows a representative fluorescent image of FITC-labeled c-Kit PMO antisense uptake. Specifically, the c Kit antisense PMO molecules were initially concentrated in intracellular vesicles (endosomes) at 3 hours in about 70% of the cells, and distributed within the cytoplasm at 66 hours. By contrast, no uptake was observed for control FITC-labeled proteins such as antibodies. Significantly, PMO oligomers were distributed within the entire cytoplasm 10 and nuclei of treated cells at 66 hours (see FIG. 3A, lower-right panel “D”).

Therefore, the introduced PMO antisense oligomers were determined to be stable over substantial time periods in DMVEC. Significantly, stable staining (FITC) was observed for up to 10 days without any toxic effects. Moreover, the PMO oligomers were readily taken up by DMVEC and distributed within the cytosol.

Proof of principal for target validation; silencing of c-Kit gene expression. The efficacy of the PMO antisense strategy for gene expression silencing in the above-described KSHV-infected DMVEC system was demonstrated using a specific FITC-labeled PMO targeting the start codon of c-Kit (5′-CGCCTCTCATCGCGGTAGCTGCG-3′; SEQ ID NO:21), a protein previously shown by applicants to induce focus formation in KSHV-infected DMVEC (Moses, et al., J. Virology 76:8383-99, 2002.).

Specifically, DMVEC were infected with KSHV, plated in 35 mm dishes and allowed to grow to about 90% confluence. For treatment, KSHV-infected cells were treated with the anti-c-Kit PMO-antisense oligomer-EPEI delivery reagent complex and incubated for 3 hours at 37° C. in serum-free medium to allow for oligomer uptake. A titration experiment testing a range of different oligomer/EPEI volumes was used to determine that loading 1.25 nmol oligomer with 2.5 μl EPEI reagent per 35 mm dish allowed efficient antisense uptake without non-specific EPEI-induced toxicity. Control (mock-treated) DMVEC cultures were loaded with EPEI reagent and sterile water or sterile water alone. Upon removal of the oligomer-EPEI solution, cell monolayers were rinsed in serum-free medium fed with complete medium and examined daily for one week by phase microscopy for evidence of phenotypic change.

FIG. 3A (panels “A,” “B” and “C”) shows that treatment with c-Kit PMO antisense (SEQ ID NO:21) resulted in restoring contact-inhibited growth of KSHV-infected DMVECs. Specfically, FIG. 3A (upper-left panel “A”) shows that during the week of post-loading culture, untreated KSHV-infected DMVECs approached confluence and were maintained in a post-confluent state. Such untreated DMVEC exhibited loss of contact inhibition and the capacity to grow in disorganized, multi-layered foci that were evident by day 6 post-loading (FIG. 3A, upper-left panel “A”). Likewise, cells cultured with 2.5 μl EPEI alone (treatment control) showed similar focus formation (FIG. 3A, upper-right panel “B”). Significantly, cells loaded with 1.25 nmol of the c-Kit antisense PMO oligomer and 2.5 μl EPEI (treated cells) did not develop foci, and maintained a quiescent contact-inhibited monolayer (FIG. 3A, lower-left panel “C”).

As described above, a direct role of c-Kit over-expression in DMVEC morphologic alteration has been previously demonstrated (Moses, et al., J. Virology 76:8383-99, 2002.). Therefore, the blockade of spindle cell, and foci formation observed herein confirms that the c-Kit antisense PMO oligomer was substantially effective in inhibiting c-Kit expression/function.

FIG. 3B shows evidence that despite expression in some cells of c-kit protein, the cell cultures treated with c-Kit antisense PMO oligomer (SEQ ID NO:21) did not progress to spindle cell and foci formation (see phase contrast images of FIG. 3A, lower-left panel “C”).

Validation of KSHV-induced gene sequences. TABLE 2 shows the validation results for thirteen induced genes identified in the experiments of EXAMPLE 2 herein above. For seven of the induced genes, suppression by sequence-specific PMO antisense oligonucleotides led to inhibitory effects (either full or intermediate inhibition) on KSHV-induced spindle cell formation in DMVEC, including two novel genes and an orphan G-protein coupled receptor. Silencing of seven of the genes (RDC-1 (GPCR RDC1), IGFBP2 (insulin-like growth factor binding protein 2), FLJ14103 (hypothetical protein FLJ14103), Neuritin, KIT (c-KIT), LOX (lysyl oxidase preprotein) and Nov (nov precursor)) resulted in fully reversed spindle cell formation, while intermediate inhibitory effects were seen for three of the genes (KIAA0367 (KIAA0367 protein), INSR (Insulin receptor) and ANGPTL2 (angiopoietin-like 2 precursor)). The specific PMO antisense oligomers used in these experiments for silencing the KSHV-induced gene sequences are also shown in TABLE 4, along with corresponding SEQ ID NOS.

TABLE 2

Validated Gene Targets; suppression (silencing) of particular
KSHV-induced genes prevented or significantly inhibited KSHV-
induced spindle cell formation.

| | | |
|---|---|---|
| | | Extent of PMO- |
| | | induced Inhibition |
| | | of Spindle Cell |
| GENE | PMO Antisense Sequence (5′ to 3′) | Formation |

| | | | |
|---|---|---|---|
| RDC-1 | GAAGAGATGCAGATCCATCGTTCTG | (SEQ ID NO:15) | full |
| IGFBP2 | GGCAGCCCACTCTCTCGGCAGCATG | (SEQ ID NO:16) | full |
| FLJ14103 | GGCTCCATCTTGGGCTCTGGGCTCC | (SEQ ID NO:17) | full |
| KIAA0367 | GTCAGTTTACTCATGTCATCTATTG | (SEQ ID NO:18) | intermediate |
| Neuritin | TTAACTCCCATCCTACGTTTAGTCA | (SEQ ID NO:19) | full |
| INSR | GGGTCTCCTCGGATCAGGCGCG | (SEQ ID NO:20) | intermediate |
| KIT | CGCCTCTCATCGCGGTAGCTGCG | (SEQ ID NO:21) | full |
| IFACTOR | AGCTTCATGTTGGAGGTGTTCG | (SEQ ID NO:22) | none |
| LMO2 | GCCGAGGACATTGGGGAGGGAGGCG | (SEQ ID NO:23) | none |
| MFAP3 | TGAATAAGCAACAATGTAGCTTCAT | (SEQ ID NO:24) | none |
| LOX | GGAGCACGGTCCAGGCGAAGCGCAT | (SEQ ID NO:31) | full |
| NOV | AGCTCGTGCTCTGCACACTCTGCAT | (SEQ ID NO:32) | full |
| ANGPTL2 | AGCATGTCACGCACAGTGGCCTCAT | (SEQ ID NO:33) | intermediate |

TABLE 3 summarizes GenBank mRNA and EST accession numbers for particular KSHV-induced genes, including for the ten validated gene sequences listed in TABLE 2. Gene names, Unigene clusters (from build #153), and GenBank accession numbers for these validated sequences are as assigned by the National Center for Biotechnology Information (NCBI), and are incorporated by reference herein, including all splice and allelic variants of these mRNA sequences.

TABLE 3
GenBank accession numbers for particular KSHV-induced genes, including for the RDC1, IGFBP2, FLJ14103,
KIAA0367, Neuritin, INSR, KIT, LOX, NOV and ANGPTL2 gene sequences validated herein.

| | Unigene | | |
|---|---|---|---|
| GENE | Cluster | Accession Numbers; mRNAs | Accession Numbers; ESTs |
| RDC-1 | Hs.23016 | BI460261 | BI767134, BM921366, BM925428, |
| | | | BM458484, R27256, AI954295, |
| | | | AA205847, AA197246, AI633054 |
| IGFBP2 | Hs.162 | BC004312, M35410, NM_000597, | BE382548, BM564454, BM928278, |
| | | BC009902, BC012769, X16302 | BM545072, BI830342, BE382760, |
| | | | BE313151, BF981949, BM548711 |
| FLJ14103 | Hs.98321 | AK024165 | BI818834, T75260, R38645, AI796127, |
| | | | AI095506, W61099, W63748, AI554899, |
| | | | AA689489, AI631711 |
| KIAA0367 | Hs.23311 | AB002365, BC022571, AL834213 | BI457935, BI552977, BG706827, |
| | | | R21961, R25052, R45391, H05195, |
| | | | H05155, R25051, R45390 |
| Neuritin | Hs.103291 | AF136631, BC002683, | BI918095, BI548839, BI602117, BI915704, |
| | | NM_016588, AJ420483, AK093824 | BE897829, BI824717, BG714127, BQ231718, |
| | | | BF970432, BF966251 |
| INSR | Hs.89695 | X02160, M10051, NM_000208 | AA860814, AA486513, AA485908, H03917, |
| | | | AI738814, AA613904, AA632501, AA632558, |
| | | | AA632596, W52906 |
| KIT | Hs.81665 | NM_000222, X06182 | BF966487, AI567686, AI567693, AI674108, |
| | | | AI308810, N20798, AA873164, AI017093, |
| | | | H10570, R35401 |
| IFACTOR | Hs.36602 | NM_000204, BC020718, J02770 | BM924043, BF132103, BG435910, BG431258, |
| | | | BG568130, BG401433, BG426851, BG566266, |
| | | | BI761434, BQ277394 |
| LMO2 | Hs.184585 | NM_005574, BC034041, | BI764252, BM808939, BG715963, BG505616, |
| | | X61118, AF257211 | R60732, AI337730, AW005586, AI687026, |
| | | | H10900, AI979150 |
| MFAP3 | Hs.28785 | AL049404, NM_005927, | BG531421, AI684093, AI933971, |
| | | BC026244, AK000358 | H60952, H61526, H99277, AI874390, |
| | | | R95175, AI452602, R13620 |
| LOX | Hs.102267 | AF039291.1, NM_002317.3, | N26939.1, H99075.1, AW005592.1, AI761085.1, |
| | | M94054.1, S78694.1, S45875.1 | AA599304.1, AI075382.1, AI022363.1, |
| | | | AI075456.1, AI335739.1, AA099452.1 |
| NOV | Hs.235935 | NM_002514.2, X96584.1, | H15316.1, R25930.1, AI920781.1, AA081850.1, |
| | | BC015028.1, AY082381.1 | AI055954.1, AA604355.1, R41819.1, |
| | | | AI923336.1, H29804.1, H29805.1 |
| ANGPTL2 | Hs.8025 | NM_012098.1, AF125175.1, | AA255567.1, AA617726.1, AI677659.1, |
| | | BC012368.1, AK075026.1, | AI934310.1, T77327.1, R38293.1, R51659.1, |
| | | AK074726.1, AF007150.1 | R51569.1, R47836.1, R51427.1 |

Inhibition of KSHV-induced cellular proliferation by PMO antisense inhibition. KSHV-infected DMVEC, as described above under EXAMPLE 1, lose the characteristic contact-inhibition displayed by DMVEC, and proliferate in response to virally-induced regulatory signals. Therefore, in addition to the inhibition/reversion of spindle-cell formation, further validation of KSHV-related cellular gene targets was achieved by determining whether silencing of particular KSHV-induced gene sequences resulted in the inhibition of KSHV-induced DMVEC proliferation. As shown below, PMO-mediated gene silencing resulted in the inhibition of KSHV-induced DMVEC proliferation, and these results correlated with the ability of the respective PMOs to inhibit spindle cell formation (phenotypic inhibition).

Proliferation assays, and loading of cells with PMOs. Proliferation of KSHV-infected DMVEC was quantified using an XTT (2,3-bis-(2-methoxy-4-nitro-5-sulfophenyl)-2H-tetrazolium-5-carboxanilide, disodium salt)-based assay. KSHV-infected cells were added to Primaria 96-well trays (Becton Dickinson) at 1×10⁴ or 5×10⁴ cells/well. XTT (Roche, Molecular Biochemicals, Indianapolis, Ind.) was added 48 hours later according to the manufacturer's instructions. Absorbance was read after 4 to 6 hours on a microplate reader.

Briefly, cells were plated in 96-well trays at a density approaching confluence (5×10⁴ cells per well) in 100 μl of complete medium. PMOs were loaded the following day in a total of 100 μl (0.5 μl PMO, 0.5 μl EPEI, 49 μl H₂O and 50 μl serum free medium) with reagent mixing as described by the manufacturer (GeneTools). Controls included a FITC PMO control oligonucleotide, EPEI only or H₂O only. Each variable was performed in quadruplicate. Fresh complete medium was replaced 4 hours after loading. Cells were cultured for 4 days to allow for multi-layered cell growth post-confluence in the absence of any growth inhibition. XTT was added on day 4 of culture and the a bsorbance read 4 hrs later on a microplate reader. Cell proliferation (growth) values are given as percentage inhibition values, relative to cells without PMO, which are adjusted to 100%.

TABLE 4 (center column) shows the extent of inhibition of KSHV-induced proliferation by specific PMO antisense inhibition of target genes (left column) as measured by XTT cellular proliferation assays. Corresponding phenotype inhibition values (extent of inhibition of spindle cell formation) are also shown (right column), based on experiments as outlined in EXAMPLE 2, herein above.

TABLE 4
Target gene-specific PMO antisense treatment; comparison
between the extent of inhibition of KSHV-induced proliferation,
and corresponding phenotype inhibition values.

| | | Phenotype Inhibition |
|---|---|---|
| | Growth Inhibition | (inhibition of spindle |
| GENE | (% of control) | cell formation) |
| IGFBP2 | 55% | Full |
| c-Kit | 50% | Full |
| RDC-1 | 43% | Full |
| Neuritin | 29% | Full |
| KIAA0367 | 28% | Intermediate |
| INSR | 26% | Intermediate |
| I-Factor | 12% | None |
| MFAP | 11% | None |
| Osteopontin | 4% | None |
| LOX | | Full |
| NOV | | Full |
| ANGPTL2 | | Intermediate |

Consistent with the above-described results for inhibition of spindle formation, PMO antisense oligonucleotide inhibition (silencing) of the validated targets, including c-Kit, RDC-1, IGFB-2, Neurtitin, KIAA0367 and INSR resulted in substantial inhibition of KSHV-induced cellular proliferation.

By contrast, silencing of other KSHV-induced gene sequences, such as MFAP, I-Factor and Osteopontin resulted in relatively little or no significant inhibition of KSHV-induced cellular proliferation. Significantly, these results are consistent with PMO antisense results disclosed herein above, which excluded these KSHV-induced gene sequences from the validated target pool.

To further support and illustrate the correspondence between the extent of inhibition of KSHV-induced proliferation and corresponding phenotype inhibition values (full inhibition, intermediate inhibition and no inhibition of spindle formation) as summarized in TABLE 4, FIGS. 5A, 5B, 5C and 5D show representative fields of KSHV-infected DMVEC treated with PMOs as indicated, and visualized by CD31 staining.

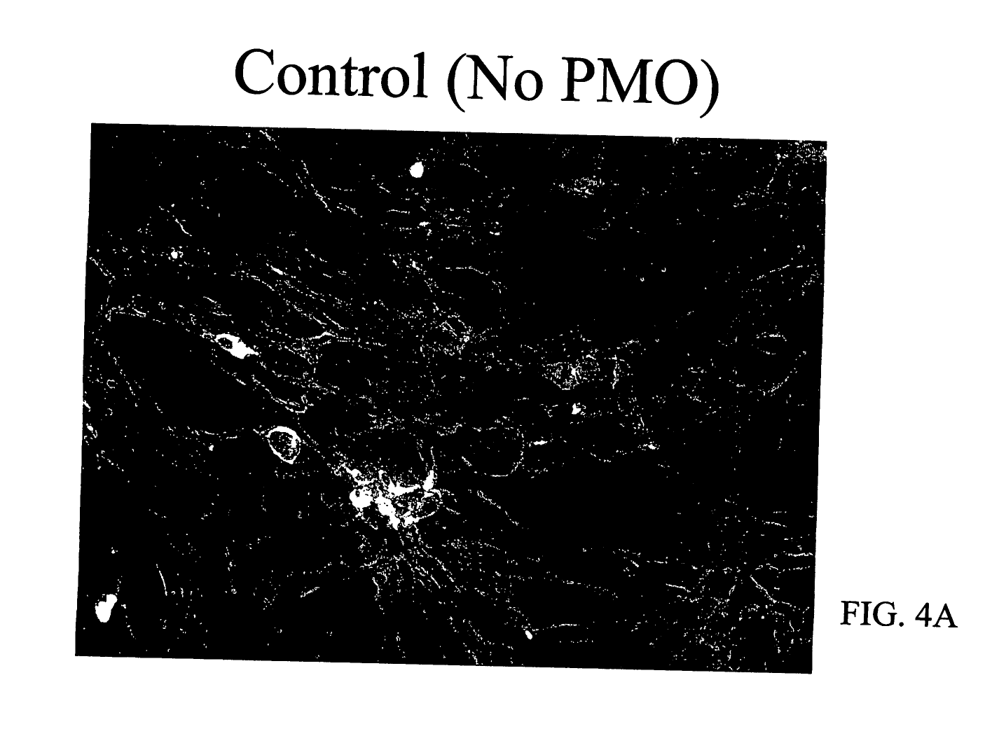

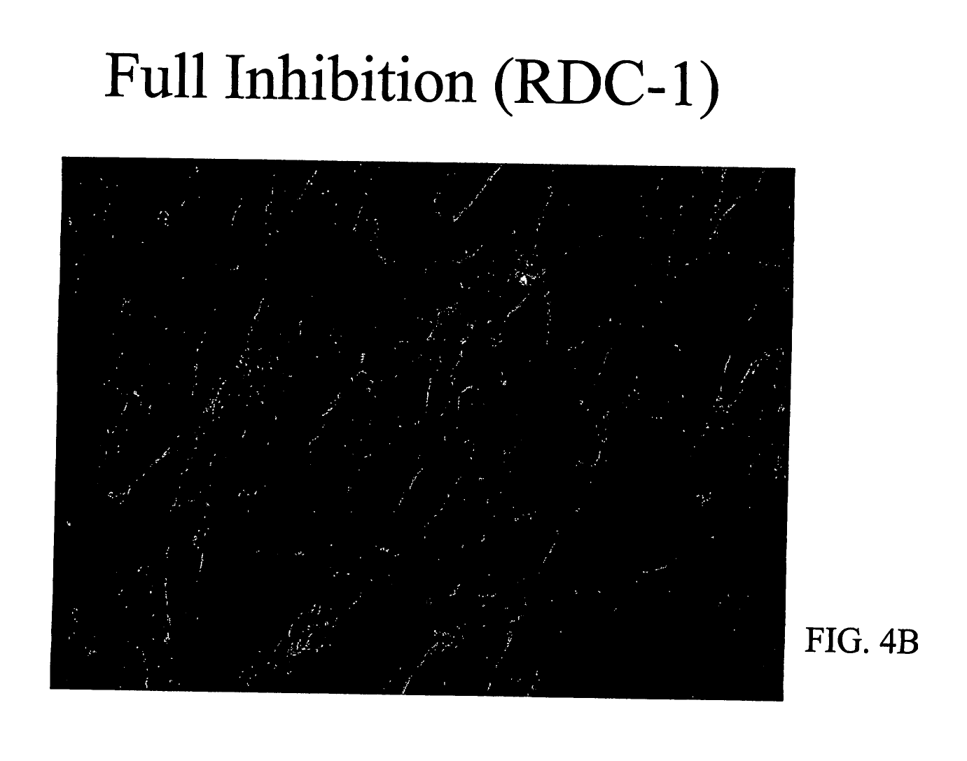

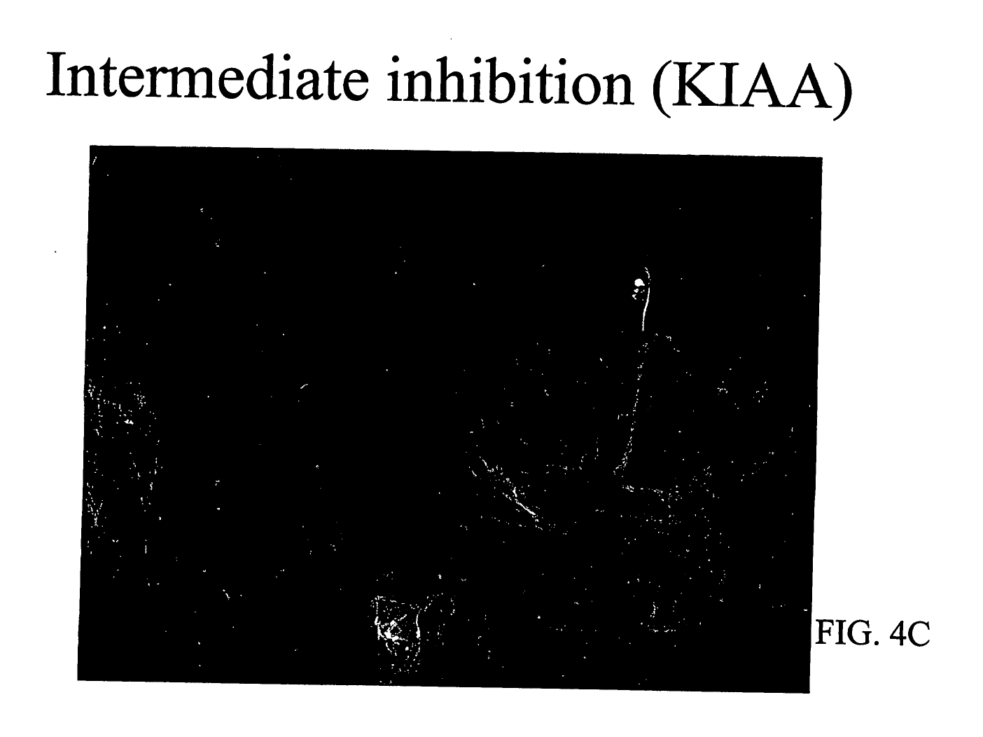

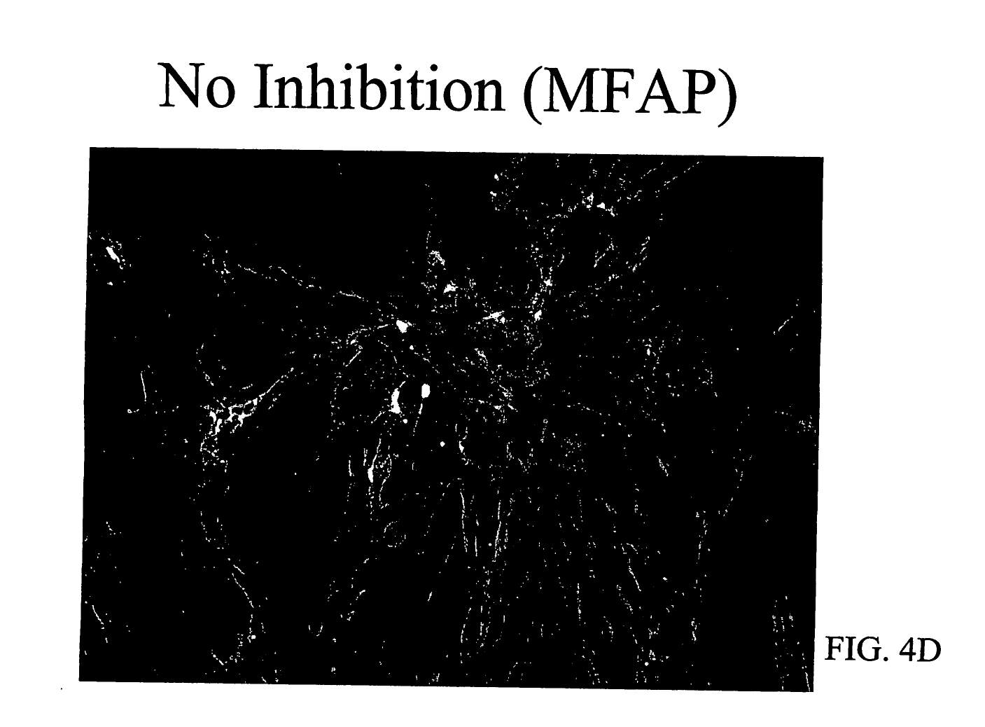

Specifically, FIG. 4A shows representative control (no PMO oligonucleotides) KSHV-infected DMVEC cultured as described herein above, and corresponds to 100% proliferation as presented in the growth inhibition assays summarized in TABLE 4.

FIG. 4B illustrates representative RDC-1-specific PMO-treated KSHV-infected DMVEC, and corresponds to the 43% growth inhibition value (full phenotypic inhibition) as presented in TABLE 4.

FIG. 4C illustrates representative KIAA0367-specific PMO-treated KSHV-infected DMVEC, and corresponds to the 28% growth inhibition value (intermediate phenotypic inhibition) as presented in TABLE 4.

FIG. 4D illustrates representative MFAP-specific PMO-treated KSHV-infected DMVEC, and corresponds to the 11% growth inhibition value (no phenotypic inhibition) as presented in TABLE 4.

Therefore, according to the present invention, the extent of PMO-mediated inhibition of KSHV-induced proliferation (% growth inhibition) correlates with the corresponding phenotype inhibition values (full, intermediate and no inhibition).

KSHV-induced genes excluded as therapeutic targets by PMO antisense validation protocol. The above Examples show that with respect to particular identified KSHV-induced genes (e.g., I-FACTOR, LMO2 and MFAP3), treatment of KSHV-infected DMVEC with the respective antisense PMO oligonucleotides had little or no affect on KSHV-induced spindle cell formation, despite the effectiveness of such antisense agents in mediating silencing of the respective gene sequences. This was not unexpected, because KSHV-related modulation of some cellular genes would reasonably be expected to be either ancillary to, or downstream from the regulatory cascades leading to spindle cell formation.

Significantly, the identification of KSHV-induced gene sequences which, upon silencing, have no effect on spindle formation provides internal (apart from the use of particular control is PMO antisense molecules, etc.) confirmation that the inventive gene-silencing mediated preclusion of spindle cell formation is not mediated through ancillary or non-sequence-specific secondary effects of the respective PMO antisense molecules.

Therefore, data presented herein describes, teaches and supports the use of sequence-specific PMO antisense oligomers, inter alia, for (i) validation of therapeutic ‘targets’; that is, for identification of KSHV-induced cellular gene products required for KSHV-induced cellular phenomena (e.g. spindle cell formation, transformation, angiogenesis, cancer, etc.), and (ii) as effective, non-toxic inhibitors of such validated therapeutic targets for modulation of KSHV infection and treatment of KSHV-induced proliferative disorders such as cancer. This utility is especially valuable where the particular gene products otherwise lack suitable art-recognized small molecule inhibitors.

Additionally, in view of deficiencies in the prior art teachings, these data emphasize the significance of functional validation of KSHV-induced gene sequences, according to the present invention to provide targets, compositions and methods having utility for blocking KSHV infection and for treating cancer.

### EXAMPLE 4

### A Novel NUDE Mouse Model for Kaposi's Sarcoma Pathogenesis

KSHV studies in vitro. Applicants have herein developed an in vitro system in which DMVEC are transformed to spindle cells that form 3-dimensional growth foci when infected with KSHV, and have used DNA microarray analysis to identify cellular genes whose expression patterns are significantly altered by virus infection. Further, applicants have herein shown that silencing the virus-induced expression of certain cellular genes with antisense oligonucleotides leads to inhibition of spindle cell formation and foci development in the described in vitro cell culture model. According to the present invention, cellular genes inappropriately activated by KSHV infection contribute to cancer formation and are novel therapeutic targets for KS treatment.

Spindle cells cultured from KS tumors do not stably maintain the KSHV genome if KS tissue explants are cultured ex vivo (Aluigi et al., Res Virol 147(5):267-75, 1996; and Ambroziak et al., Science 268(5210):582-3, 1995). Thus, the development of endothelial cell-based in vitro models of KSHV infection that accurately reflect both the virus lifecycle and the disease phenotype is important for understanding KS tumorigenesis. Applicants were the first to successfully describe such a system based on infection of dermal microvascular endothelial cells (DMVEC) (Moses et al., J. Virol. 73(8):6892-6902, 1999). In this model, the majority of DMVEC become latently infected, cells develop a phenotype reminiscent of KS spindle cells, and lose contact inhibition when cultured post confluence (see also Ciufo, et al., J Virol 75(12):5614-26, 2001; and Lagunoff, et al., J Virol 76(5):2440-8, 2002).

In vivo studies. A limited number of murine models for KS have previously been described. KS cell lines isolated from AIDS/KS patients have been used to produce tumors of human origin in immunodeficient mice (Lunardi-Iskandar, et al., J Natl. Cancer Inst. 5:974-981, 1995; and Albini et al., FASAEB J. 13:647-655, 1999). These human KS cell lines have also been used to promote the growth of angioproliferative lesions of mouse origin by secretion of factors such as VEGF and bFGF (Ensoli, et al., Nature 371:674-676, 1994; and Samaniego, et al., J Immunol. 158:1887-1897, 1997). However, these models are somewhat limited by the fact that while the utilized KS cell lines induce angiogenic lesions, these cells do not maintain the KSHV genome over the long-term.

Recently, KS-like tumors have been generated in mice transgenic for the avian leucosis virus (ALV) receptor, TVA; the mice were infected with ALV vectors expressing KSHV genes (Montaner, et al., Cancer Cell. 3:23-36, 2003). However, this model is limited by the fact that the induced tumors are of mouse origin and were induced via retroviral vectors encoding KSHV oncogenes.

Therefore, there is a need in the art to create tumors of human origin that maintain the entire KSHV genome, and thus more accurately reflect the cellular and viral interactions occurring in KS lesions. There is a need in the art for an in vivo model that can be used to directly examine the role of virus-induced cellular proteins in driving tumor establishment and/or growth. There is a need in the art for an in vivo model system to screen and test novel KS drugs. There is a need in the art for an in vivo model system wherein the cells contain the KSHV genome, so that inhibitors of virus replication as well as gene expression can be screened/tested.

Irradiation model; mice were irradiated to impair immune function. In particular embodiments of the present invention, BALB/c mice were subjected to irradiation to temporarily decrease immune function and ablate the tumor rejection response. Mock- and KSHV-infected DMVEC (3×10⁶ cells/injection) were suspended in serum-free culture medium, mixed with 0.2 ml (1:1) of matrigel and injected subcutaneously into the tail base. 10 days later, mice were humanely euthanized according to an OHSU IACUC-approved protocol and matrigel plugs were excised. One half of each plug was placed into tissue culture for phase microscopy observation after which it was used for extraction of cellular DNA and PCR for the KSHV Bam330 fragment to verify maintenance of the KSHV genome. The other half was embedded in paraffin, sectioned and stained with a rabbit anti-human polyclonal antibody against heme-oxygenase 1, a cellular protein induced by KSHV infection of DMVEC and implicated in the angiogenic process (McAllister, et al., Blood In press, 2004).

Results. Matrigel plugs excised from the control mouse injected with mock-infected DMVEC contained only degenerating cell clumps. In obvious contrast, KSHV-infected cells had developed into a distinct vascular network running through the 3-dimensional matrigel matrix. 233 bp of KSHV ORF26 (Bam300 fragment) was amplified exclusively from DNA extracted from within the KSHV-infected DMVEC matrigel plug, indicating maintenance of the KSHV genome. Finally, immunohistochemical staining of paraffin-embedded matrigel sections revealed reactivity to human HO-1 in vascular threads within the KSHV-infected matrigel sections.

Therefore, according to the present invention, KSHV-infected DMVEC showed a preferential tendency to survive and undergo angiogenesic growth in immunodeficient (irradiated) mice.

Novel Nude mouse model. According to the present invention, applicants' KSHV-infected DMVEC model has further utility to induce KS-like tumors in immunodeficient mice.

According to the present invention, a nude mouse model for KS is developed by implanting KSHV-transformed DMVEC into immunodeficient (nude) mice.

According to the present invention, DMVEC are treated prior to implantation into nude mice to inhibit the expression of virus-induced genes, whereby the tumorigenic potential of the treated implants is evaluated.

According to the present invention, the use of nude mice, allows for more robust tumor growth, and allows for the efficient growth of KSHV-infected human cells in the mouse model, development of KS like tumors, and further validation of anti KS therapies.

Specifically, according to particular embodiments of the present invention, Nude mice (Foxn1^{nu}) on a BALB/cByJ genetic background are obtained from The Jackson Laboratory (Bar Harbor, Me.). Because the forkhead box N1 gene mutation disrupts thymic function, nude mice exhibit T cell deficiency with some defects in B cell development. The activity of macrophages, antigen presenting cells and NK cells is unaffected, and reduces susceptibility to murine pathogens. Nude mice have been widely used for the growth of human tumors, and the lack of hair allows visualization of sub-cutaneous tumors.

According to the present invention, mice receive subcutaneous injections at the tail base, where the injection material consists of KSHV infected human dermal microvascular endothelial cells (DMVEC) (3×10⁶ cells/injection) that are suspended in serum-free culture medium and mixed with 0.2 ml (1:1) of matrigel. DMVEC are infected with KSHV at least two weeks prior to inoculation, to allow establishment of latent infection in the majority of cells (Moses et al., J. Virol. 73(8):6892-6902, 1999; and Moses, et al., J. Virol. 76(16):8383-8399, 2002). Negative controls include animals injected with uninfected DMVEC in matrigel or with matrigel alone. As a positive control, the fibrosarcoma HT1080 (ATCC # CRL-12012) that readily forms tumors in nude mice is used.

In some experiments, DMVEC are loaded with antisense oligonucleotides (PMOs) to inhibit expression of specific cellular genes 24 hours prior to inoculation (Moses, et al., Ann NY Acad Sci 975:1-12, 2002). Briefly, cells are incubated with a PMO-loading reagent complex for three hours, rinsed and cultured overnight prior to resuspension in matrigel and inoculation. Parallel cultures are maintained in vitro to verify PMO uptake and efficiency of gene silencing. Alternatively, siRNA agents and methods are used to inhibit expression of specific cellular sequences.

According to the present invention, mice are observed and weighed daily. Caliper measurements of tumor size are recorded daily. At days 7 and 14 post-inoculation, mice are euthanized. Lesions at the site of inoculation are macroscopically examined, excised, measured and weighed. If no lesions are present, equivalent tissue areas around the injection site a re excised. Excised tissue is divided into thirds and is treated as follows: (i) fixed in formalin for histologic examination following H&E staining; (ii) frozen in OCT for immunohistochemistry; (iii) processed for RNA extraction and pPCR analysis. Protein and mRNA evaluations include cellular and viral targets.

Additional organs such as spleen and draining lymph node are processed and analyzed. Mice are examined for metastases to the gut, liver and kidney and such tissues are harvested if warranted.

All animals are euthanized at the pre-assigned times. Animals are euthanized immediately if they exhibit any signs of undue tumor burden including: a tumor that exceeding 2 cm or 10% of body weight; ulceration of tumor, tumor impeding ambulation or ability to obtain food or water; if the animal exhibits signs or pain or distress; or if the animal is cachexic or moribund. A protocol for these studies is approved by the OSHU IACUC Protocol # A924.

According to the present invention, mice inoculated with HT1080 fibrosarcoma cells form tumors and serve as a positive control. According to the present invention, mice inoculated with KSHV-infected DMVEC develop tumors at the injection site within 5-7 days, whereas no tumors develop in mice inoculated with uninfected DMVEC or with matrigel alone.

According to the present invention, mice inoculated with KSHV-DMVEC in which expression of KSHV genes has been inhibited by PMO treatment (or siRNA treatment) show different degrees of tumor inhibition, depending on the relative importance of the cellular gene that is targeted. A central role for c-Kit in KS transformation has been demonstrated in vitro, and, according to the present invention, tumor formation is inhibited in vivo when c-Kit expression is inhibited. According to the present invention, the performance of other PMOs in this in vivo system likewise confirms the role of the targeted cellular gene in KS tumorigenesis, and further validates the therapeutic approach.

According to the present invention, mice are inoculated with KSHV-DMVEC in which PMO treatment (or siRNA treatment) is used to inhibit expression of at least one KSHV-induced cellular gene sequence selected from the group disclosed herein consisting of: RDC-1 (GPCR RDC1); IGFBP2 (insulin-like growth factor binding protein 2); FLJ14103 (hypothetical protein FLJ14103); Neuritin; KIT (c-KIT); LOX (lysyl oxidase preprotein); Nov (nov precursor); KIAA0367 (KIAA0367 protein); INSR (Insulin receptor); and ANGPTL2 (angiopoietin-like 2 precursor), wherein inhibition of tumors, relative to controls, is shown, and whereby the targeted sequences are further validated and whereby therapeutic utility is further confirmed.

1. A method for identification of agents or compounds useful to modulate KSHV infection, comprising:
(a) contacting one or more KSHV-infected cells with a test agent or compound;
(b) measuring in the one or more cells, and using a suitable assay, expression of a validated KSHV-induced cellular gene or gene product, wherein a validated gene or gene product is a gene or gene product the expression of which is required, at least to some extent, for KSHV infection or KSHV-mediated effects on cellular proliferation and phenotype; and
(c) determining, relative to one or more control cells not contacted with the test agent or compound, whether the test agent or compound inhibits the validated gene or gene product expression, whereby agents or compounds that inhibit the validated gene or gene product expression are identified as agents or compounds useful to modulate KSHV infection.

2. The method of claim 1, wherein measuring expression of a validated KSHV-induced cellular gene or gene product is by measuring the presence or amount at least one of the corresponding mRNA or the protein product encoded thereby.

3. The methods of any one of claims 1 or 2, further comprising testing of the agents or compounds that inhibit the validated KSHV-induced cellular gene or gene product expression for the ability to modulate at least one of KSHV infection, or KSHV-mediated effects on cellular proliferation or phenotype.

4. The methods of any one of claims 1, 2 or 3, wherein the KSHV-infected cells are KSHV-infected dermal microvascular endothelial cells (DMVEC).

5. The method of any one of claims 14, comprising measuring the expression of a plurality of validated KSHV-induced cellular genes or gene products.

6. The method of any one of claims 1-5, wherein at least one of measuring or determining comprises use of high-throughput microarray methods.

7. The method or assay of any one of claims 1 through 6, wherein the validated KSHV-induced cellular genes or gene products correspond to one or more nucleic acid sequences selected from the group consisting of SEQ ID NOS: 1, 3, 5, 7, 9, 11, 13, 25, 27 and 29, for the RDC-1, IGFBP2, FLJ14103, KIAA0367, Neuritin, INSR, KIT (c-kit), LOX, NOV and ANGPTL2 cDNA sequences, respectively.

8. The methods of any one of claims 1 through 6, wherein the validated KSHV-induced cellular genes or gene products correspond to one or more amino acid sequences selected from the group consisting of SEQ ID NOS:2, 4, 6, 8, 10, 12, 14, 26, 28 and 30, for the RDC-1, IGFBP2, FLJ14103, KIAA0367, Neuritin, INSR, KIT (c-kit), LOX, NOV and ANGPTL2 protein sequences, respectively.

9. A diagnostic or prognostic assay for KSHV infection, comprising:
(a) obtaining a cell sample from a subject having, or suspected of having KSHV;
(b) measuring in the sample, and using a suitable assay, expression of a validated KSHV-induced cellular gene or gene product, wherein a validated gene or gene product is a gene or gene product the expression of which is required, at least to some extent, for KSHV infection; and
(c) determining, based on the measuring, and relative to that of non-KSHV-infected control cells, whether expression of the validated KSHV-induced cellular gene or gene product is induced, whereby a diagnosis or prognosis is, at least in part, afforded.

10. The assay of claim 9, comprising measuring the expression of a plurality of validated KSHV-induced cellular genes or gene products.

11. The assay of any one of claims 9 or 10, wherein at least one of measuring or determining comprises use of high-throughput microarray methods.

12. The assay of any one of claims 9, 10 or 11, wherein the validated KSHV-induced cellular genes or gene products correspond to one or more nucleic acid sequences selected from the group consisting of SEQ ID NOS:1, 3, 5, 7, 9, 11, 13, 25, 27 and 29, for the RDC-1, IGFBP2, FLJ14103, KIAA0367, Neuritin, INSR, KIT (c-kit), LOX, NOV and ANGPTL2 cDNA sequences, respectively.

13. The assay of any one of claims 9, 10 or 11, wherein the validated KSHV-induced cellular genes or gene products correspond to one or more amino acid sequences selected from the group consisting of SEQ ID NOS:2, 4, 6, 8, 10, 12, 14, 26, 28 and 30, for the RDC-1, IGFBP2, FLJ14103, KIAA0367, Neuritin, INSR, KIT (c-kit), LOX, NOV and ANGPTL2 protein sequences, respectively.

14. A method of inhibiting at least one of: KSHV-induced cellular gene expression or encoded biological activity; KSHV infection; or KSHV-mediated effects on cellular proliferation and phenotype, comprising introducing into, or expressing within a KSHV-infected human cell at least one of an antisense, siRNA or ribozyme agent specific for a validated KSHV-induced cellular gene sequence, and in an amount sufficient to inhibit, at least to some extent, expression of the validated KSHV-induced cellular gene sequence, wherein a validated KSHV-induced cellular gene sequence is a nucleic acid sequence the expression of which is required, at least to some extent, for the KSHV-induced cellular gene expression or encoded biological activity, the KSHV infection, or the KSHV-mediated effects on cellular proliferation and phenotype.

15. The method of claim 14, wherein inhibiting the KSHV-mediated effects on cellular proliferation and phenotype comprises inhibiting proliferation or development of cancer cells.

16. The method of any one of claims 14 or 15, wherein the validated KSHV-induced cellular gene sequence is that corresponding to a nucleic acid sequence selected from the group consisting of SEQ ID NOS:1, 3, 5, 7, 9, 11, 13, 25, 27 and 29, for the RDC-1, IGFBP2, FLJ14103, KIAA0367, Neuritin, INSR, KIT (c-kit), LOX, NOV and ANGPTL2 cDNA sequences, respectively.

17. The method of any one of claims 14-16, wherein the antisense agent specific for a validated KSHV-induced cellular gene sequence comprises a nucleic acid sequence of at least 18 contiguous bases in length that is complementary to, or hybridizes under moderately stringent or stringent conditions to a sequence selected from the group consisting of SEQ ID NOS:1, 3, 5, 7, 9, 11, 13, 25, 27, 29, and sequences complementary thereto.

18. The method of any one of claims 14-17, wherein the antisense agent specific for a validated KSHV-induced cellular gene sequence comprises a nucleic acid sequence selected from the group consisting of SEQ ID NOS:15-24, 31-32 and 33.

19. The method of any one of claims 14-18, wherein the validated KSHV-induced cellular gene sequence-specific antisense agent comprises a Phosphorodiamidate Morpholino Oligomers (PMO) antisense oligonucleotide specific for the validated KSHV-induced cellular gene sequence.

20. A method for inhibiting or treating KSHV-infection in a subject, or for treating KSHV-related neoplastic disease, comprising administering to the subject a therapeutically effective amount of at least one of an antisense, siRNA or ribozyme agent specific for a validated KSHV-induced cellular gene sequence, wherein the validated KSHV-induced cellular gene sequence is a nucleic acid sequence the expression of which is required, at least to some extent, for the KSHV-infection or the KSHV-related neoplastic disease.

21. The method of claim 20, wherein the validated KSHV-induced cellular gene sequence is that corresponding to a nucleic acid sequence selected from the group consisting of SEQ ID NOS:1, 3, 5, 7, 9, 11, 13, 25, 27 and 29, for the RDC-1, IGFBP2, FLJ14103, KIAA0367, Neuritin, INSR, KIT (c-kit), LOX, NOV and ANGPTL2 cDNA sequences, respectively.

22. The method of any one of claims 20 or 21, wherein the antisense agent specific for a validated KSHV-induced cellular gene sequence comprises a nucleic acid sequence of at least 18 contiguous bases in length that is complementary to, or hybridizes under moderately stringent or stringent conditions to a sequence selected from the group consisting of SEQ ID NOS:1, 3, 5, 7, 9, 11, 13, 25, 27, 29, and sequences complementary thereto.

23. The method of any one of claims 20-22, wherein the antisense agent specific for a validated KSHV-induced cellular gene sequence comprises a nucleic acid sequence selected from the group consisting of SEQ ID NOS:15-24, 31-32 and 33.

24. The method of any one of claims 20-23, wherein the validated KSHV-induced cellular gene sequence-specific antisense agent comprises a Phosphorodiamidate Morpholino Oligomers (PMO) antisense oligonucleotide specific for the validated KSHV-induced cellular gene sequence.

25. Use of an inhibitor of validated KSHV-induced gene or gene product expression to prepare a medicament for modulating at least one of KSHV infection, KSHV-mediated effects on cellular proliferation or phenotype, or KSHV-related neoplastic disease, and wherein the inhibitor comprises at least one of an antisense, siRNA or ribozyme agent specific for the validated KSHV-induced gene or gene product.

26. The use of claim 25, wherein the validated KSHV-induced cellular genes or gene products correspond to one or more nucleic acid sequences selected from the group consisting of SEQ ID NOS:1, 3, 5, 7, 9, 11, 13, 25, 27 and 29, for the RDC-1, IGFBP2, FLJ14103, KIAA0367, Neuritin, INSR, KIT (c-kit), LOX, NOV and ANGPTL2 cDNA sequences, respectively.

27. The use of claim 25, wherein the validated KSHV-induced cellular genes or gene products correspond to one or more amino acid sequences selected from the group consisting of SEQ ID NOS:2, 4, 6, 8, 10, 12, 14, 26, 28 and 30, for the RDC-1, IGFBP2, FLJ14103, KIAA0367, Neuritin, INSR, KIT (c-kit), LOX, NOV and ANGPTL2 protein sequences, respectively.

28. The use of any one of claims 25, 26 or 27, wherein the inhibitor of validated KSHV-induced gene or gene product expression comprises an antisense agent specific to the validated KSHV-induced gene or gene product.

29. The use of any one of claims 25-28, wherein the antisense agent specific for a validated KSHV-induced cellular gene sequence comprises a nucleic acid sequence of at least 18 contiguous bases in length that is complementary to, or hybridizes under moderately stringent or stringent conditions to a sequence selected from the group consisting of SEQ ID NOS:1, 3, 5, 7, 9, 11, 13, 25, 27, 29, and sequences complementary thereto.

30. The use of any one of claims 25-29, wherein the antisense agent specific for a validated KSHV-induced cellular gene sequence comprises a nucleic acid sequence selected from the group consisting of SEQ ID NOS:15-24, 31-32 and 33.

31. The use of any one of claims 25-30, wherein the validated KSHV-induced cellular gene sequence-specific antisense agent comprises a Phosphorodiamidate Morpholino Oligomers (PMO) antisense oligonucleotide specific for the validated KSHV-induced cellular gene sequence.

32. An antisense oligonucleotide, siRNA agent, or a ribozyme agent comprising a sequence of about 10 to about 35 contiguous nucleotides in length that is complementary to, or hybridizes under moderately stringent or stringent conditions to a sequence selected from the group consisting of SEQ ID NOS:1, 3, 5, 7, 9, 11, 13, 25, 27, 29, and sequences complementary thereto, wherein the antisense oligonucleotide, siRNA agent, or a ribozyme agent is effective to inhibit cellular expression, at least to some degree, of the respective KSHV-induced human cellular gene product.

33. A recombinant expression vector, comprising a transcriptional initiation region and a sequence encoding a KSHV-induced gene-specific antisense oligonucleotide, siRNA agent, or ribozyme agent a sequence of about 10 to about 35 contiguous nucleotides in length that is complementary to, or hybridizes under moderately stringent or stringent conditions to a sequence selected from the group consisting of SEQ ID NOS:1, 3, 5, 7, 9, 11, 13, 25, 27, 29, and sequences complementary thereto.

34. An in vivo mouse model for KSHV infection and KSHV-related conditions, comprising introduction of KSHV-infected human dermal microvascular endothelial cells (DMVEC) into a immunodeficient NUDE mouse strain.

34. The mouse model of claim 34, wherein the NUDE mouse strain is Foxn1^{nu} on a BALB/cByJ genetic background.

35. The mouse model of any one of claims 34 or 35, wherein KS-like tumors are induced by introduction of KSHV-infected human dermal microvascular endothelial cells (DMVEC).


SEQUENCE LISTING (33 sequences)

SEQ ID NO: 1 - DNA, 2035 bp, homo sapiens
  CDS (152)..(1240)
tgcaagtctgcagccagcagagctcacagttgttgcaaagtgctcagcactaagggagccagcgcacagcacagccaggaaggcgagcgagcccagccagcccagccagcccagccagcccggaggtcatttgattgcccgcctcagaacgatggatctgcatctcttcgactactcagagccagggaacttctcggacatcagctggccatgcaacagcagcgactgcatcgtggtggacacggtgatgtgtcccaacatgcccaacaaaagcgtcctgctctacacgctctccttcatttacattttcatcttcgtcatcggcatgattgccaactccgtggtggtctgggtgaatatccaggccaagaccacaggctatgacacgcactgctacatcttgaacctggccattgccgacctgtgggttgtcctcaccatcccagtctgggtggtcagtctcgtgcagcacaaccagtggcccatgggcgagctcacgtgcaaagtcacacacctcatcttctccatcaacctcttcggcagcattttcttcctcacgtgcatgagcgtggaccgctacctctccatcacctacttcaccaacacccccagcagcaggaagaagatggtacgccgtgtcgtctgcatcctggtgtggctgctggccttctgcgtgtctctgcctgacacctactacctgaagaccgtcacgtctgcgtccaacaatgagacctactgccggtccttctaccccgagcacagcatcaaggagtggctgatcggcatggagctggtctccgttgtcttgggctttgccgttcccttctccattatcgctgtcttctacttcctgctggccagagccatctcggcgtccagtgaccaggagaagcacagcagccggaagatcatcttctcctacgtggtggtcttccttgtctgctggctgccctaccacgtggcggtgctgctggacatcttctccatcctgcactacatccctttcacctgccggctggagcacgccctcttcacggccctgcatgtcacacagtgcctgtcgctggtgcactgctgcgtcaaccctgtcctctacagcttcatcaatcgcaactacaggtacgagctgatgaaggccttcatcttcaagtactcggccaaaacagggctcaccaagctcatcgatgcctccagagtctcagagacggagtactctgccttggagcagagcaccaaatgatctgccctggagaggctctgggacgggtttacttgtttttgaacagggtgatgggccctatggttttctagagcaaagcaaagtagcttcgggtcttgatgcttgagtagagtgaagaggggagcacgtgccccctgcatccattctctctttctcttgatgacgcagctgtcatttggctgtgcgtgctgacagttttgcaacaggcagagctgtgtcgcacagcagtgctgtgcgtcagagccagctgaggacaggcttgcctggacttctgtaagataggattttctgtgtttcctgaattttttatatggtgatttgtatttaaattttaagactttattttctcactattggtgtaccttataaatgtatttgaaagttaaatatattttaaatattgtttgggaggcatagtgctgacatatattcagagtgttgtagttttaaggttagcgtgacttcagttttgactaaggatgacactaattgttagctgttttgaaattatatatatataaatatatataaatatataaatatatgccagtcttggctgaaatgttttatttaccatagttttatatctgtgtggtgttttgtaccggcacgggatatggaacgaaaactgctttgtaatgcagtttgtgacattaatagtattgtaaagttacattttaaaataaacaaaaaactgttctggactgcaaatctgcacacacaacgaacagttgcatttcagagagttctctcaatttgtaagttatttttttttaataaagatttttgtttcct

SEQ ID NO: 2 - protein, 362 aa, homo sapiens
MDLHLFDYSEPGNFSDISWPCNSSDCIVVDTVMCPNMPNKSVLLYTLSFIYIFIFVIGMIANSVVVWVNIQAKTTGYDTHCYILNLAIADLWVVLTIPVWVVSLVQHNQWPMGELTCKVTHLIFSINLFGSIFFLTCMSVDRYLSITYFTNTPSSRKKMVRRVVCILVWLLAFCVSLPDTYYLKTVTSASNNETYCRSFYPEHSIKEWLIGMELVSVVLGFAVPFSIIAVFYFLLARAISASSDQEKHSSRKIIFSYVVVFLVCWLPYHVAVLLDIFSILHYIPFTCRLEHALFTALHVTQCLSLVHCCVNPVLYSFINRNYRYELMKAFIFKYSAKTGLTKLIDASRVSETEYSALEQSTK

SEQ ID NO: 3 - DNA, 1421 bp, homo sapiens
  CDS (115)..(1092)
ggcgagggaggaggaagaagcggaggaggcggctcccgcgctcgcagggccgtgccacctgcccgcccgcccgctcgctcgctcgcccgccgcgccgcgctgccgaccgccagcatgctgccgagagtgggctgccccgcgctgccgctgccgccgccgccgctgctgccgctgctgctgctgctactgggcgcgagtggcggcggcggcggggcgcgcgcggaggtgctgttccgctgcccgccctgcacacccgagcgcctggccgcctgcgggcccccgccggttgcgccgcccgccgcggtggccgcagtggccggaggcgcccgcatgccatgcgcggagctcgtccgggagccgggctgcggctgctgctcggtgtgcgcccggctggagggcgaggcgtgcggcgtctacaccccgcgctgcggccaggggctgcgctgctatccccacccgggctccgagctgcccctgcaggcgctggtcatgggcgagggcacttgtgagaagcgccgggacgccgagtatggcgccagcccggagcaggttgcagacaatggcgatgaccactcagaaggaggcctggtggagaaccacgtggacagcaccatgaacatgttgggcgggggaggcagtgctggccggaagcccctcaagtcgggtatgaaggagctggccgtgttccgggagaaggtcactgagcagcaccggcagatgggcaagggtggcaagcatcaccttggcctggaggagcccaagaagctgcgaccaccccctgccaggactccctgccaacaggaactggaccaggtcctggagcggatctccaccatgcgccttccggatgagcggggccctctggagcacctctactccctgcacatccccaactgtgacaagcatggcctgtacaacctcaaacagtgcaagatgtctctgaacgggcagcgtggggagtgctggtgtgtgaaccccaacaccgggaagctgatccagggagcccccaccatccggggggaccccgagtgtcatctcttctacaatgagcagcaggaggctcgcggggtgcacacccagcggatgcagtagaccgcagccagccggtgcctggcgcccctgccccccgcccctctccaaacaccggcagaaaacggagagtgcttgggtggtgggtgctggaggattttccagttctgacacacgtatttatatttggaaagagaccagcaccgagctcggcacctccccggcctctctcttcccagctgcagatgccacacctgctccttcttgctttccccgggggaggaagggggttgtggtcggggagctggggtacaggtttggggagggggaagagaaatttttatttttgaacccctgtgtcccttttgcataagattaaaggaaggaaaagt

SEQ ID NO: 4 - protein, 325 aa, homo sapiens
MLPRVGCPALPLPPPPLLPLLLLLLGASGGGGGARAEVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENHVDSTMNMLGGGGSAGRKPLKSGMKELAVFREKVTEQHRQMGKGGKHHLGLEEPKKLRPPPARTPCQQELDQVLERISTMRLPDERGPLEHLYSLHIPNCDKHGLYNLKQCKMSLNGQRGECWCVNPNTGKLIQGAPTIRGDPECHLFYNEQQEARGVHTQRMQ

SEQ ID NO: 5 - DNA, 2502 bp, homo sapiens
  CDS (76)..(624)
ctctttggccaagccctgcctctgtacagcctcgagtggacagccagaggctgcagctggagcccagagcccaagatggagccccagctggggcctgaggctgccgccctccgccctggctggctggccctgctgctgtgggtctcagccctgagctgttctttctccttgccagcttcttccctttcttctctggtgccccaagtcagaaccagctacaattttggaaggactttcctcggtcttgataaatgcaatgcctgcatcgggacatctatttgcaagaagttctttaaagaagaaataagatctgacaactggctggcttcccaccttggactgcctcccgattccttgctttcttatcctgcaaattactcagatgattccaaaatctggcgccctgtggagatctttagactggtcagcaaatatcaaaacgagatctcagacaggaaaatctgtgcctctgcatcagccccaaagacctgcagcattgagcgtgtcctgcggaaaacagagaggttccagaaatggctgcaggccaagcgcctcacgccggacctggtgcaggactgtcaccagggccagagagaactaaagttcctgtgtatgctgagataacaccagtgaaaaagcctggcatggagcccagcactgagaacttccagaaagtgttagccttctcccaactgtgttataccaaccacattttcaaatagtaatcattaaagaggcttctgcatcaaaccttcacatgcagctcccatgccaccctccagaattcaccaacacacaggcccaccagcaacaggctacctttgcacaatattctctgatgacaactccaaagccccggctctttccaccacactgtggtcccctagatggggctgttgctgagcccaccccaatccagatgtgatccccctgtgatctacttctggcaagattctcagtctggacaggtcttccctatgagatagaacctgataaggagctagggcaattctgacaacattaccaaaggcccacataacttctaaattttggtctggtctgaaggaaaacctgttctcgccctagtgatggatgaactctcttatctctggcttctagagggaaaaaaaaagcatacctcttttactttttaagtacctccatcagagtcatgaaatcacctgtcaagactatctatcttttatgtttccattctggtaagaactctttaaatgaggacactgctgattgctggtgatgttttttgagcaaacactcgggggtatggatgaaagccaatcgcaggtcaaatgactccttggggaagctacttctcctctattcagatttcactaaaatcttccaagatgaaagcaaatctagatttcggtcttcattgctgtccatttttgtaatgaacgagtgtttttcctttagctagtgtatcaggcagggttctaccagagaaacagaaccagtaggagatacatatacatgtccagatttatttcaaagaattgatttacatgattgtggggattggcaagtccaaaatccatatggtaggcctgcaatctgtaaacctttgggcaggagctgatgctgtagtttgcagatagaattccttgttccttaaaaaaatctgtttttgttcttaagggctttgaatgattggatcaggcccacccagattacctagataatctcttttacttaaagtaaactgattgtaggtgctaatcacatctatgaaatgccttcacagcaacacctagattagcattcaattgaataactggggaatacagcctagccaagttgacacataaaattaaccatcacagcaacatgcctgctaaattttatcgaccgtcttcagactgttaaggattgtggtagagaactgtgacagccactctcagcatcaccctgaaccaaaggcccctatcaagtaacaatatagccaagcaaaattccagtcaatagagacattgactggttggctggcttcccaagggatagcaccagacaagaaatgcaaggatgaggaaaccaggcacgggagagggaggggcaacagaggtccagggtttggttatctttttatttttcactgggaggtggtaagttagccctgttgcccatgtatgcagatgggagaagtgatttagaaactccaaagcaattggtaatccccaaaatgggtgtatctggtttgaaatgaaaccttattttattggaaatggttggtttcccaattctgtttgccattggccaatataattgtgggtttgcacatggccagcacatgccaaacagaagtagacaaaggtctcactctgtaagtgggaccttggggaggagctgcctccatcataaagggaggggttagtaaaaatggtctcttaagcctgttcctgctacagttatagaggttgctcagaaccttctcagcaaatatagcagttatctattgttgtgtattaaaccatttcaacacat

SEQ ID NO: 6 - protein, 182 aa, homo sapiens
MEPQLGPEAAALRPGWLALLLWVSALSCSFSLPASSLSSLVPQVRTSYNFGRTFLGLDKCNACIGTSICKKFFKEEIRSDNWLASHLGLPPDSLLSYPANYSDDSKIWRPVEIFRLVSKYQNEISDRKICASASAPKTCSIERVLRKTERFQKWLQAKRLTPDLVQDCHQGQRELKFLCMLR

SEQ ID NO: 7 - DNA, 5668 bp, homo sapiens
  CDS (163)..(2475)
gctttgtttgatggtgatccacatttatccacagagaatcctgccttggttcctgatgctttgctagcctcagacacttgtctggatataagcgaagctgcctttgaccacagtttcagcgatgcctcaggtctcaacacatccacgggaacaatagatgacatgagtaaactgacattatccgaaggccatccggaaacgccagttgatggggacctagggaagcaagatatctgctcatctgaagcctcgtggggtgattttgaatatgatgtaatgggccagaatatcgatgaagatttactgagagagcctgaacacttcctgtatggtggtgaccctcctttggaggaagattctctgaagcagtcgctggcaccgtacacacctccctttgatttgtcttatctcacagaacctgcccagagtgctgaaacaatagaggaagctgggtctccagaggatgaatctctgggatgcagagcagcagagatagtgctttctgcacttcctgatcgaagaagtgagggaaaccaggctgagaccaaaaacagactgcctggatcccagctggctgtgctgcatattcgtgaagaccctgagtccgtttatttgccggtaggagcaggctccaacattttgtctccatcaaacgttgactgggaagtagaaacagataattctgatttaccagcaggtggagacataggaccaccaaatggtgccagcaaggaaatatcagaattggaagaagaaaaaacaattcctaccaaagagcctgagcagataaaatcagaatacaaggaagaaagatgtacagagaagaatgaagatcgtcatgcactacacatggattacatacttgtaaaccgtgaagaaaattcacactcaaagccagagacctgtgaagaaagagaaagcatagctgaattagaattgtatgtaggttccaaagaaacagggctgcagggaactcagttagcaagcttcccagacacatgtcagccagcctccttaaatgaaagaaaaggtctctctgcagagaaaatgtcttctaaaagcgatacgagatcatcttttgaaagccctgcacaagaccagagttggatgttcttgggccatagtgaggttggtgatccatcactggatgccagggactcagggcctgggtggtctggcaagactgtggagccgttctctgaactcggcttgggtgagggtccccagctgcagattctggaagaaatgaagcctctagaatctttggcactagaggaagcctctggtccagtcagccaatcacagaagagtaagagccgaggcagggctggcccggatgcagttacgttgcaggctgtcacccatgacaatgaatgggaaatgctttcaccacagcctgttcagaaaaacatgatccctgacacggaaatggaggaggagacagagttccttgagctcggaaccaggatatcaagaccaaatggactactgtcagaggatgtaggaatggacatcccctttgaagagggcgtgctgagtcccagtgctgcagacatgaggcctgaacctcctaattctctggatcttaatgacactcatcctcggagaatcaagctcacagccccaaatatcaatctttctctggaccaaagtgaaggatctattctctctgatgataacttggacagtccagatgaaattgacatcaatgtggatgaacttgatacccccgatgaagcagattcttttgagtacactggccatgatcccacagccaacaaagattctggccaagagtcagagtctattccagaatatacggccgaagaggaacgggaggacaaccggctttggaggacagtggtcattggagaacaagagcagcgcattgacatgaaggtcatcgagccctacaggagagtcatttctcacggaggatactatggggacggtctaaatgccatcattgtgtttgccgcctgttttctgccagacagcagtcgggcggattaccactatgtcatggaaaatcttttcctatatgtaataagtactttagagttgatggtagctgaagactatatgattgtgtacttgaatggtgcaaccccaagaaggaggatgccagggctaggctggatgaagaaatgctaccagatgattgacagacggttgaggaagaatttgaaatcattcatcattgttcatccatcttggttcatcagaacaatccttgctgtgacacgaccttttataagttcaaaattcagcagtaaaattaaatatgtcaatagcttatcagaactcagtgggctgatcccaatggattgcatccacattccagagagcatcatcaaactggatgaagaactgagggaagcatcagaggcagctaaaactagctgcctttacaatgatccagaaatgtcttctatggagaaggatattgacttgaagctgaaagaaaagccttagttggccatgctggaagaagaggatgcttttctggttcatggttctgttgaaacatatctacctgaaagagacagggctgatgttacctttttccactttgcactacctggtgccattctaaatttctaaggggaaaaatagaaagtttgtttactcttaagatattttatgaaattgtgtgtactttcctattttgccaattatgtgcctcaaagattttagttgagccttagcaagaaagtaggaccttccatttcaatacttcattaacacggtgtagtgatactttgtcccttagactggtgtttaccagtaagatacctttaatccactgttaagtatgagtggatttgtttccatagattagctggatttccttttggtgattgcattaggtttaaagtacacaggtctcaactctccccaggaaagtttcccctgtttgactccacctttaaaatcctaagcctgactaggacagccacaaaccacacaaggtgtaaaaccatcatcagctaagtgcccgttttgttcttgtttaccagaatctcctttaacttctcaaagggaagccgggctttctaatccacgtcaactttattttagttgtcaaattgggcattatattttatgtaaattggtcttttaacatcattttcctgatgaatgttggtgaccaccacattgtgaaatttaagaatccgtgttgcatgtttggtagctctctgagtttcaggccataaactcagctccagaggttaccttttaagtgccaagaactcaagtgcaaggtggcctactcaaaaatcatttggtagcattcagttattcatgaattcctctctcgcatgcattataaaaagtgatctgctttaaaacaccgtaatctgatcataggcttaaaattaaatatgagtattactttcatgtacaaaatatttcctttatagtcttcatatgccctttaaaatgccaacaagatttcaagtctgtaggcctctagtgaggtggggtggcaaaccacagctaagtctcgctcaccactgcaagctaagaatggtttttacattttgggttggaaaaattttttttgaatatttcatgacacatgaaaattattcaaatgttagtgccgataaataaagtggtactgaaacacagccacacaaacttgtttttgtactgtctacagctactttcacactacagccgcagagctgagcagttcagcagaccgtatgtcccacaatgcctaaaacattgactatgtttacagaaaaagtttgctgacccctgctctagcaaacgcatcctttcctactccaccccaatttgtatttagatagtttctctaacagaacggacaaatgaggctgcaaactaatttatttttgtcaaaaatcaatgttttgacatccacagacagtgaaataaaagaaatggcttgctgaaaaacatgaggagtcctagccacaaaatcactgcttaggttgcaattgccaaaatgaagccttcttagaagcacttctttagtatatacaggtgttggctgaagtccgtgcctcactctgggaaccattcttagtctccagtgtctcctattacaaagaagctggcagaaataaaaatgaaggggtgagagcggttccaccctagtctcatggtggaaaattcattggggagagctgtccaggatatttggagtcctgggtagaaggagcttgtaactactttaaagtcgacatctttgcacaggtgattgagtttctctgacctcattgcttcacctctgtctcctcccgtccttccgcacgtgcccacacacacgcagttcagccctctttcctccataagcctccatcgttttctcttttctcctcttgatcctttcaagcgagtatcttgttgaattgtatgttctgttggatctcctccttcataacatctggcttgttggacagaaaaaccctacagcccaccccctcccacagcccacctccacttttgaaagcccaaattacacctctcccagaacacagtgttgacgtaaatacagttacccaatattcctgtttgttcacctatttgctactttcactcagtagcatcccattttgtaaaatgaattccatggtcaccctgtcacaggaagtaatgaaaaatccagtgttcagtgtagtggtgcaaacctgagggcatagagctgttcatagagggctcttgttatagccaaacagacacagcaacaatctcaccatttatatatatatttttaacttgtccagctcatctatggaaaactactcaggtggtatgctgtttgaagcctcatcttcctacatgaaaattatgggcatttgtcccaatgattttgtttcagctgttctgtaggctgcataaccactctgatatttaggtatctgctattttattatcttaaaagacaaattaatttaattgcatgtgctagggaaaagctaccatgtacattcaccccaagtaaatagaatcctagatgaatcctagaaaaataatccctaagcagataggtagacagaggtaaacattcacatgatttagctctctagctcttgcactctgaacattcttgctttggttctgacttctgggaactgctttgcatttctcctatagatctgtagttaagggaaccaaggggtcattggggcaaaagcattgtttctcaaagctccttgattaagagaaagaacagaaatttgcacagaagatagtgtcaaggagtgagaaagtttgtttgagggcagtagctcagtgtggaagaaaatcctgaagtttctgttgaagccatacaatgttctatggggttactctctaagacattctctgaggtgtgtgaggaagtcactactcctagcctttgttaagatgtaattttaaatattcagttatggtactatgtttgcaactctcgtcttatcacaatgcctcagtagtttgttcccttagaaacatttagatgtgcacaaattaatcttttatatatctaaaggtttttctatcatgcattggattgctcagaataaagtgtctgttagacttcgttttggtaaataaattctccataatgtagattaataatataaaagtctttaatgacacaatatatctatatagcctcactgtataattcagaaataaaaattgattctgc

SEQ ID NO: 8 - protein, 770 aa, homo sapiens
MSKLTLSEGHPETPVDGDLGKQDICSSEASWGDFEYDVMGQNIDEDLLREPEHFLYGGDPPLEEDSLKQSLAPYTPPFDLSYLTEPAQSAETIEEAGSPEDESLGCRAAEIVLSALPDRRSEGNQAETKNRLPGSQLAVLHIREDPESVYLPVGAGSNILSPSNVDWEVETDNSDLPAGGDIGPPNGASKEISELEEEKTIPTKEPEQIKSEYKEERCTEKNEDRHALHMDYILVNREENSHSKPETCEERESIAELELYVGSKETGLQGTQLASFPDTCQPASLNERKGLSAEKMSSKSDTRSSFESPAQDQSWMFLGHSEVGDPSLDARDSGPGWSGKTVEPFSELGLGEGPQLQILEEMKPLESLALEEASGPVSQSQKSKSRGRAGPDAVTLQAVTHDNEWEMLSPQPVQKNMIPDTEMEEETEFLELGTRISRPNGLLSEDVGMDIPFEEGVLSPSAADMRPEPPNSLDLNDTHPRRIKLTAPNINLSLDQSEGSILSDDNLDSPDEIDINVDELDTPDEADSFEYTGHDPTANKDSGQESESIPEYTAEEEREDNRLWRTVVIGEQEQRIDMKVIEPYRRVISHGGYYGDGLNAIIVFAACFLPDSSRADYHYVMENLFLYVISTLELMVAEDYMIVYLNGATPRRRMPGLGWMKKCYQMIDRRLRKNLKSFIIVHPSWFIRTILAVTRPFISSKFSSKIKYVNSLSELSGLIPMDCIHIPESIIKLDEELREASEAAKTSCLYNDPEMSSMEKDIDLKLKEKP

SEQ ID NO: 9 - DNA, 1589 bp, Homo sapiens
  CDS (169)..(597)
gtctcttcctcgctccctctctttctctcctccctctgccttcccagtgcataaagtctctgtcgctcccggaacttgttggcaatgcctattttttggctttcccccgcgttctctaaactaactatttaaaggtctgcggtcgcaaatggtttgactaaacgtaggatgggacttaagttgaacggcagatatatttcactgatcctcgcggtgcaaatagcgtatctggtgcaggccgtgagagcagcgggcaagtgcgatgcggtcttcaagggcttttcggactgtttgctcaagctgggcgacagcatggccaactacccgcagggcctggacgacaagacgaacatcaagaccgtgtgcacatactgggaggatttccacagctgcacggtcacagcccttacggattgccaggaaggggcgaaagatatgtgggataaactgagaaaagaatccaaaaacctcaacatccaaggcagcttattcgaactctgcggcagcggcaacggggcggcggggtccctgctcccggcgttcccggtgctcctggtgtctctctcggcagctttagcgacctggctttccttctgagcgtggggccagctccccccgcgcgcccacccacactcactccatgctcccggaaatcgagaggaagatccattagttctttggggacgttgtgattctctgtgatgctgaaaacactcatataggattgtgggaaatcctgattctcttttttatttcgtttgatttcttgtgttttatttgccaaatgttaccaatcagtgagcaagcaagcacagccaaaatcggacctcagctttagtccgtcttcacacacaaataagaaaacggcaaacccaccccattttttaattttattattattaattttttttgttggcaaaagaatctcaggaacggccctgggccacctactatattaatcatgctagtaacatgaaaaatgatgggctcctcctaataggaaggcgaggagaggagaaggccaggggaatgaattcaagagagatgtccacggccgaaacatacggtgaataattcacgctcacgtcgttcttccacagtatcttgttttgatcatttccactgcacatttctcctcaagaaaagcgaaaggacagactgttggctttgtgtttggaggataggagggagagagggaaggggctgaggaaatctctggggtaagagtaaaggcttccagaagacatgctgctatggtcactgaggggttagctttatctgctgttgttgatgcatccgtccaagttcactgcctttattttccctcctccctcttgttttagctgttacacacacagtaatacctgaatatccaacggtatagatcacaagggggggatgttaaatgttaatctaaaatatagctaaaaaaagattttgacataaaagagccttgattttaaaaaaaaaagagagagagatgtaatttaaaaagtttattataaattaaattcagcaaaaaaagatttgctacaaagtatagagaagtataaaataaaagttattgtttgaaaaaaaaaaaaaaaaaa

SEQ ID NO: 10 - protein, 142 aa, Homo sapiens
MGLKLNGRYISLILAVQIAYLVQAVRAAGKCDAVFKGFSDCLLKLGDSMANYPQGLDDKTNIKTVCTYWEDFHSCTVTALTDCQEGAKDMWDKLRKESKNLNIQGSLFELCGSGNGAAGSLLPAFPVLLVSLSAALATWLSF

SEQ ID NO: 11 - DNA, 5180 bp, Homo sapiens
  CDS (49)..(4161)
accgggagcgcgcgctctgatccgaggagaccccgcgctcccgcagccatgggcaccgggggccggcggggggcggcggccgcgccgctgctggtggcggtggccgcgctgctactgggcgccgcgggccacctgtaccccggagaggtgtgtcccggcatggatatccggaacaacctcactaggttgcatgagctggagaattgctctgtcatcgaaggacacttgcagatactcttgatgttcaaaacgaggcccgaagatttccgagacctcagtttccccaaactcatcatgatcactgattacttgctgctcttccgggtctatgggctcgagagcctgaaggacctgttccccaacctcacggtcatccggggatcacgactgttctttaactacgcgctggtcatcttcgagatggttcacctcaaggaactcggcctctacaacctgatgaacatcacccggggttctgtccgcatcgagaagaacaatgagctctgttacttggccactatcgactggtcccgtatcctggattccgtggaggataattacatcgtgttgaacaaagatgacaacgaggagtgtggagacatctgtccgggtaccgcgaagggcaagaccaactgccccgccaccgtcatcaacgggcagtttgtcgaacgatgttggactcatagtcactgccagaaagtttgcccgaccatctgtaagtcacacggctgcaccgccgaaggcctctgttgccacagcgagtgcctgggcaactgttctcagcccgacgaccccaccaagtgcgtggcctgccgcaacttctacctggacggcaggtgtgtggagacctgcccgcccccgtactaccacttccaggactggcgctgtgtgaacttcagcttctgccaggacctgcaccacaaatgcaagaactcgcggaggcagggctgccaccagtacgtcattcacaacaacaagtgcatccctgagtgtccctccgggtacacgatgaattccagcaacttgctgtgcaccccatgcctgggtccctgtcccaaggtgtgccacctcctagaaggcgagaagaccatcgactcggtgacgtctgcccaggagctccgaggatgcaccgtcatcaacgggagtctgatcatcaacattcgaggaggcaacaatctggcagctgagctagaagccaacctcggcctcattgaagaaatttcagggtatctaaaaatccgccgatcctacgctctggtgtcactttccttcttccggaagttacgtctgattcgaggagagaccttggaaattgggaactactccttctatgccttggacaaccagaacctaaggcagctctgggactggagcaaacacaacctcaccatcactcaggggaaactcttcttccactataaccccaaactctgcttgtcagaaatccacaagatggaagaagtttcaggaaccaaggggcgccaggagagaaacgacattgccctgaagaccaatggggaccaggcatcctgtgaaaatgagttacttaaattttcttacattcggacatcttttgacaagatcttgctgagatgggagccgtactggccccccgacttccgagacctcttggggttcatgctgttctacaaagaggccccttatcagaatgtgacggagttcgacgggcaggatgcatgtggttccaacagttggacggtggtagacattgacccacccctgaggtccaacgaccccaaatcacagaaccacccagggtggctgatgcggggtctcaagccctggacccagtatgccatctttgtgaagaccctggtcaccttttcggatgaacgccggacctatggggccaagagtgacatcatttatgtccagacagatgccaccaacccctctgtgcccctggatccaatctcagtgtctaactcatcatcccagattattctgaagtggaaaccaccctccgaccccaatggcaacatcacccactacctggttttctgggagaggcaggcggaagacagtgagctgttcgagctggattattgcctcaaagggctgaagctgccctcgaggacctggtctccaccattcgagtctgaagattctcagaagcacaaccagagtgagtatgaggattcggccggcgaatgctgctcctgtccaaagacagactctcagatcctgaaggagctggaggagtcctcgtttaggaagacgtttgaggattacctgcacaacgtggttttcgtccccaggccatctcggaaacgcaggtcccttggcgatgttgggaatgtgacggtggccgtgcccacggtggcagctttccccaacacttcctcgaccagcgtgcccacgagtccggaggagcacaggccttttgagaaggtggtgaacaaggagtcgctggtcatctccggcttgcgacacttcacgggctatcgcatcgagctgcaggcttgcaaccaggacacccctgaggaacggtgcagtgtggcagcctacgtcagtgcgaggaccatgcctgaagccaaggctgatgacattgttggccctgtgacgcatgaaatctttgagaacaacgtcgtccacttgatgtggcaggagccgaaggagcccaatggtctgatcgtgctgtatgaagtgagttatcggcgatatggtgatgaggagctgcatctctgcgtctcccgcaagcacttcgctctggaacggggctgcaggctgcgtgggctgtcaccggggaactacagcgtgcgaatccgggccacctcccttgcgggcaacggctcttggacggaacccacctatttctacgtgacagactatttagacgtcccgtcaaatattgcaaaaattatcatcggccccctcatctttgtctttctcttcagtgttgtgattggaagtatttatctattcctgagaaagaggcagccagatgggccgctgggaccgctttacgcttcttcaaaccctgagtatctcagtgccagtgatgtgtttccatgctctgtgtacgtgccggacgagtgggaggtgtctcgagagaagatcaccctccttcgagagctggggcagggctccttcggcatggtgtatgagggcaatgccagggacatcatcaagggtgaggcagagacccgcgtggcggtgaagacggtcaacgagtcagccagtctccgagagcggattgagttcctcaatgaggcctcggtcatgaagggcttcacctgccatcacgtggtgcgcctcctgggagtggtgtccaagggccagcccacgctggtggtgatggagctgatggctcacggagacctgaagagctacctccgttctctgcggccagaggctgagaataatcctggccgccctccccctacccttcaagagatgattcagatggcggcagagattgctgacgggatggcctacctgaacgccaagaagtttgtgcatcgggacctggcagcgagaaactgcatggtcgcccatgattttactgtcaaaattggagactttggaatgaccagagacatctatgaaacggattactaccggaaagggggcaagggtctgctccctgtacggtggatggcaccggagtccctgaaggatggggtcttcaccacttcttctgacatgtggtcctttggcgtggtcctttgggaaatcaccagcttggcagaacagccttaccaaggcctgtctaatgaacaggtgttgaaatttgtcatggatggagggtatctggatcaacccgacaactgtccagagagagtcactgacctcatgcgcatgtgctggcaattcaaccccaacatgaggccaaccttcctggagattgtcaacctgctcaaggacgacctgcaccccagctttccagaggtgtcgttcttccacagcgaggagaacaaggctcccgagagtgaggagctggagatggagtttgaggacatggagaatgtgcccctggaccgttcctcgcactgtcagagggaggaggcggggggccgggatggagggtcctcgctgggtttcaagcggagctacgaggaacacatcccttacacacacatgaacggaggcaagaaaaacgggcggattctgaccttgcctcggtccaatccttcctaacagtgcctaccgtggcgggggcgggcaggggttcccattttcgctttcctctggtttgaaagcctctggaaaactcaggattctcacgactctaccatgtccaatggagttcagagatcgttcctatacatttctgttcatcttaaggtggactcgtttggttaccaatttaactagtcctgcagaggatttaactgtgaacctggagggcaaggggtttccacagttgctgctcctttggggcaacgacggtttcaaaccaggattttgtgttttttcgttccccccacccgcccccagcagatggaaagaaagcacctgtttttacaaattctttttttttttttttttttttgctggtgtctgagcttcagtataaaagacaaaacttcctgtttgtggaacaaaagttcgaaagaaaaaacaaaacaaaaacacccagccctgttccaggagaatttcaagttttacaggttgagcttcaagatggtttttttggttttttttttttctctcatccaggctgaaggattttttttttctttacaaaatgagttcctcaaattgaccaatagctgctgctttcatattttggataagggtctgtggtcccggcgtgtgctcacgtgtgtatgcacgtgtgtgtgtccattagacacggctgacgtgtgtgcaaagtatccatgcggagttgatgctttgggaattggctcatgaaggttcttctcaagggtgcgagctcatccccctctctccttccttcttattgactgggagactgtgctctcgacagattcttcttgtgtcagaagtctagcctcaggtttctaccctcccttcacattggtggccaagggaggagcatttcatttggagtgattatgaatcttttcaagaccaaaccaagctaggacattaaaaaaaaaaaaaagaaaaagaaagaaaaaacaaaatggaaaaaggaaaaaaaaaaagaactgagatgacagagttttgagaatatatttgtaccatattta

SEQ ID NO: 12 - protein, 1370 aa, Homo sapiens [sequence omitted]

SEQ ID NO: 13 - DNA, 5084 bp, Homo sapiens
  CDS (22)..(2952)
  variation (3101)..(3101): C and T alleles exist at this position
gatcccatcgcagctaccgcgatgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctgagcagaatcagtgtttgggtcacccctccaggaatgatctcttcttttggcttccatgatggttattttcttttctttcaacttgcatccaactccaggatagtgggcaccccactgcaatcctgtctttctgagcacactttagtggcygatgatttttgtcatcagccaccatcctattgcaaaggttccaactgtatatattcccaatagcaacgtagcttctaccatgaacagaaaacattctgatttggaaaaagagagggaggtatggactgggggccagagtcctttccaaggcttctccaattctgcccaaaaatatggttgatagtttacctgaataaatggtagtaatcacagttggccttcagaaccatccatagtagtatgatgatacaagattagaagctgaaaacctaagtcctttatgtggaaaacagaacatcattagaacaaaggacagagtatgaacacctgggcttaagaaatctagtatttcatgctgggaatgagacataggccatgaaaaaaatgatccccaagtgtgaacaaaagatgctcttctgtggaccactgcatgagcttttatactaccgacctggtttttaaatagagtttgctattagagcattgaattggagagaaggcctccctagccagcacttgtatatacgcatctataaattgtccgtgttcatacatttgaggggaaaacaccataaggtttcgtttctgtatacaaccctggcattatgtccactgtgtatagaagtagattaagagccatataagtttgaaggaaacagttaataccattttttaaggaaacaatataaccacaaagcacagtttgaacaaaatctcctcttttagctgatgaacttattctgtagattctgtggaacaagcctatcagcttcagaatggcattgtactcaatggatttgatgctgtttgacaaagttactgattcactgcatggctcccacaggagtgggaaaacactgccatcttagtttggattcttatgtagcaggaaataaagtataggtttagcctccttcgcaggcatgtcctggacaccgggccagtatctatatatgtgtatgtacgtttgtatgtgtgtagacaaatatttggaggggtatttttgccctgagtccaagagggtcctttagtacctgaaaagtaacttggctttcattattagtactgctcttgtttcttttcacatagctgtctagagtagcttaccagaagcttccatagtggtgcagaggaagtggaaggcatcagtccctatgtatttgcagttcacctgcacttaaggcactctgttatttagactcatcttactgtacctgttccttagaccttccataatgctactgtctcactgraacatttaaattttaccctttagactgtagcctggatattattcttgtagtttacctctttaaaaacaaaacaaaacaaaacaaaaaactccccttcctcactgcccaatataaaaggcaaatgtgtacatggcagagtttgtgtgttgtcttgaaagattcaggtatgttgcctttatggtttcccccttctacatttcttagactacatttagagaactgtggccgttatctggaagtaaccatttgcactggagttctatgctctcgcacctttccaaagttaacagattttggggttktgttgtcacccaagagattgttgtttgccatactttgtctgaaaaattcctttgtgtttctattgacttcaatgatagtaagaaaagtggttgttagttatagatgtctaggtacttcaggggcacttcattgagagttttgtcttgccatactttgtctgaaaaattcctttgtgtttctattgacttcaatgatagtaagaaaagtggttgttagttatagatgtctaggtacttcaggggcacttcattgagagttttgtcaatgtcttttgaatattcccaagcccatgagtccttgaaaatattttttatatatacagtaactttatgtgtaaatacataagcggcgtaagtttaaaggatgttggtgttccacgtgttttattcctgtatgttgtccaattgttgacagttctgaagaattc

SEQ ID NO: 14 - protein, 976 aa, Homo sapiens [sequence omitted]

SEQ ID NO: 15 - DNA, 25 bp, artificial sequence
  RDC-1-specific PMO antisense oligonucleotide
gaagagatgcagatccatcgttctg

SEQ ID NO: 16 - DNA, 25 bp, artificial sequence
  IGFBP-2-specific PMO antisense oligonucleotide
ggcagcccactctctcggcagcatg

SEQ ID NO: 17 - DNA, 25 bp, artificial sequence
  FLJ14103-specific PMO antisense oligonucleotide
ggctccatcttgggctctgggctcc

SEQ ID NO: 18 - DNA, 25 bp, artificial sequence
  KIAA0367-specific PMO antisense oligonucleotide
gtcagtttactcatgtcatctattg

SEQ ID NO: 19 - DNA, 25 bp, artificial sequence
  Neuritin-specific PMO antisense oligonucleotide
ttaactcccatcctacgtttagtca

SEQ ID NO: 20 - DNA, 22 bp, artificial sequence
  INSR-specific PMO antisense oligonucleotide
gggtctcctcggatcaggcgcg

SEQ ID NO: 21 - DNA, 23 bp, artificial sequence
  KIT-specific PMO antisense oligonucleotide
cgcctctcatcgcggtagctgcg

SEQ ID NO: 22 - DNA, 22 bp, artificial sequence
  IFACTOR-specific PMO antisense oligonucleotide
agcttcatgttggaggtgttcg

SEQ ID NO: 23 - DNA, 25 bp, artificial sequence
  LMO2-specific PMO antisense oligonucleotide
gccgaggacattggggagggaggcg

SEQ ID NO: 24 - DNA, 25 bp, artificial sequence
  MFAP3-specific PMO antisense oligonucleotide
tgaataagcaacaatgtagcttcat

SEQ ID NO: 25 - DNA, 1946 bp, Homo sapiens
  CDS (274)..(1527)
agacactgcccgctctccgggactccgcgccgctccccgttgccttccaggactgagaaaggggaaagggaagggtgccacgtccgagcagccgccttgactggggaagggtctgaatcccacccttggcattgcttggtggagactgagatacccgtgctccgctcgcctccttggttgaagatttctccttccctcacgtgatttgagccccgtttttattttctgtgagccacgtcctcctcgagcggggtcaatctggcaaaaggagtgatgcgcttcgcctggaccgtgctcctgctcgggcctttgcagctctgcgcgctagtgcactgcgcccctcccgccgccggccaacagcagcccccgcgcgagccgccggcggctccgggcgcctggcgccagcagatccaatgggagaacaacgggcaggtgttcagcttgctgagcctgggctcacagtaccagcctcagcgccgccgggacccgggcgccgccgtccctggtgcagccaacgcctccgcccagcagccccgcactccgatcctgctgatccgcgacaaccgcaccgccgcggcgcgaacgcggacggccggctcatctggagtcaccgctggccgccccaggcccaccgcccgtcactggttccaagctggctactcgacatctagagcccgcgaagctggcgcctcgcgcgcggagaaccagacagcgccgggagaagttcctgcgctcagtaacctgcggccgcccagccgcgtggacggcatggtgggcgacgacccttacaacccctacaagtactctgacgacaacccttattacaactactacgatacttatgaaaggcccagacctgggggcaggtaccggcccggatacggcactggctacttccagtacggtctcccagacctggtggccgacccctactacatccaggcgtccacgtacgtgcagaagatgtccatgtacaacctgagatgcgcggcggaggaaaactgtctggccagtacagcatacagggcagatgtcagagattatgatcacagggtgctgctcagatttccccaaagagtgaaaaaccaagggacatcagatttcttacccagccgaccaagatattcctgggaatggcacagttgtcatcaacattaccacagtatggatgagtttagccactatgacctgcttgatgccaacacccagaggagagtggctgaaggccacaaagcaagtttctgtcttgaagacacatcctgtgactatggctaccacaggcgatttgcatgtactgcacacacacagggattgagtcctggctgttatgatacctatggtgcagacatagactgccagtggattgatattacagatgtaaaacctggaaactatatcctaaaggtcagtgtaaaccccagctacctggttcctgaatctgactataccaacaatgttgtgcgctgtgacattcgctacacaggacatcatgcgtatgcctcaggctgcacaatttcaccgtattagaaggcaaagcaaaactcccaatggataaatcagtgcctggtgttctgaagtgggaaaaaatagactaacttcagtaggatttatgtattttgaaaaagagaacagaaaacaacaaaagaatttttgtttggactgttttcaataacaaagcacataactggattttgaacgcttaagtcatcattacttgggaaatttttaatgtttattatttacatcactttgtgaattaacacagtgtttcaattctgtaattacatatttgactctttcaaagaaatccaaatttctcatgttccttttgaaattgtagtgcaaaatggtcagtattatctaaatgaatgagccaaaatgactttgaactgaaacttttctaaagtgctggaactttagtgaaacataataataatgggtttata

SEQ ID NO: 26 - protein, 417 aa, Homo sapiens
MRFAWTVLLLGPLQLCALVHCAPPAAGQQQPPREPPAAPGAWRQQIQWENNGQVFSLLSLGSQYQPQRRRDPGAAVPGAANASAQQPRTPILLIRDNRTAAARTRTAGSSGVTAGRPRPTARHWFQAGYSTSRAREAGASRAENQTAPGEVPALSNLRPPSRVDGMVGDDPYNPYKYSDDNPYYNYYDTYERPRPGGRYRPGYGTGYFQYGLPDLVADPYYIQASTYVQKMSMYNLRCAAEENCLASTAYRADVRDYDHRVLLRFPQRVKNQGTSDFLPSRPRYSWEWHSCHQHYHSMDEFSHYDLLDANTQRRVAEGHKASFCLEDTSCDYGYHRRFACTAHTQGLSPGCYDTYGADIDCQWIDITDVKPGNYILKVSVNPSYLVPESDYTNNVVRCDIRYTGHHAYASGCTISPY

SEQ ID NO: 27 - DNA, 2389 bp, Homo sapiens
  CDS (73)..(1146)
gggaaggcgagcagtgccaatctacagcgaagaaagtctcgtttggtaaaagcgagaggggaaagcctgagcatgcagagtgtgcagagcacgagcttttgtctccgaaagcagtgcctttgcctgaccttcctgcttctccatctcctgggacaggtcgctgcgactcagcgctgccctccccagtgcccgggccggtgccctgcgacgccgccgacctgcgcccccggggtgcgcgcggtgctggacggctgctcatgctgtctggtgtgtgcccgccagcgtggcgagagctgctcagatctggagccatgcgacgagagcagtggcctctactgtgatcgcagcgcggaccccagcaaccagactggcatctgcacggcggtagagggagataactgtgtgttcgatggggtcatctaccgcagtggagagaaatttcagccaagctgcaaattccagtgcacctgcagagatgggcagattggctgtgtgccccgctgtcagctggatgtgctactgcctgagcctaactgcccagctccaagaaaagttgaggtgcctggagagtgctgtgaaaagtggatctgtggcccagatgaggaggattcactgggaggccttacccttgcagcttacaggccagaagccaccctaggagtagaagtctctgactcaagtgtcaactgcattgaacagaccacagagtggacagcatgctccaagagctgtggtatggggttctccacccgggtcaccaataggaaccgtcaatgtgagatgctgaaacagactcggctctgcatggtgcggccctgtgaacaagagccagagcagccaacagataagaaaggaaaaaagtgtctccgcaccaagaagtcactcaaagccatccacctgcagttcaagaactgcaccagcctgcacacctacaagcccaggttctgtggggtctgcagtgatggccgctgctgcactccccacaataccaaaaccatccaggcagagtttcagtgctccccagggcaaatagtcaagaagccagtgatggtcattgggacctgcacctgtcacaccaactgtcctaagaacaatgaggccttcctccaggagctggagctgaagactaccagagggaaaatgtaacctatcactcaagaagcacacctacagagcacctgtagctgctgcgccacccaccatcaaaggaatataagaaaagtaatgaagaatcacgatttcatccttgaatcctatgtattttcctaatgtgatcatatgaggacctttcatatctgtcttttatttaacaaaaaatgtaattaactgtaaacttggaatcaaggtaagctcaggatatggcttaggaatgacttactttcctgtggttttattacaaatgcaaatttctataaatttaagaaaacaagtatataatttactttgtagactgtttcacattgcactcatcatattttgttgtgcactagtgcaattccaagaaaatatcactgtaatgagtcagtgaagtctagaatcatacttaacatttcattgtacaagtattacaaccatatattgaggttcattgggaagattctctattggctccctttttgggtaaaccagctctgaacttccaagctccaaatccaaggaaacatgcagctcttcaacatgacatccagagatgactattacttttctgtttagttttacactaggaaacgtgttgtatctacagtaatgaaatgtttactaagtggactggtgtcataaactttctccatttaagacacattgactcctttccaatagaaagaaactaaacagaaaactcccaatacaaagatgactggtccctcatagccctcagacatttatatattggaagctgctgaggcccccaagttttttaattaagcagaaacagcatattagcagggattctctcatctaactgatgagtaaactgaggcccaaagcacttgcttacatcctctgatagctgtttcaaatgtgcattttgtggaattttgagaaaaatagagcaaaatcaacatgactggtggtgagagaccacacattttatgagagtttggaattattgtagacatgcccaaaacttatccttgggccataattatgaaaactcatgatcaagatatatgtgtatacatacatgtatctggtttgtcaggctacaaggtaggctgcaaaattaaatctagacattcttttaatgccaccacacgtgttccgcttctctcttttaaagtatttataaaaatataaattgtacattttgtaaaatattatgtttgatttctctacttgtcatatcactaaataaacacgattttattgctgaaaaaaaaaaaaaaaaaaa

SEQ ID NO: 28 - protein, 357 aa, Homo sapiens
MQSVQSTSFCLRKQCLCLTFLLLHLLGQVAATQRCPPQCPGRCPATPPTCAPGVRAVLDGCSCCLVCARQRGESCSDLEPCDESSGLYCDRSADPSNQTGICTAVEGDNCVFDGVIYRSGEKFQPSCKFQCTCRDGQIGCVPRCQLDVLLPEPNCPAPRKVEVPGECCEKWICGPDEEDSLGGLTLAAYRPEATLGVEVSDSSVNCIEQTTEWTACSKSCGMGFSTRVTNRNRQCEMLKQTRLCMVRPCEQEPEQPTDKKGKKCLRTKKSLKAIHLQFKNCTSLHTYKPRFCGVCSDGRCCTPHNTKTIQAEFQCSPGQIVKKPVMVIGTCTCHTNCPKNNEAFLQELELKTTRGKM

SEQ ID NO: 29 - DNA, 1518 bp, Homo sapiens
  CDS (22)..(1503)
aaccaccattttgcaaggaccatgaggccactgtgcgtgacatgctggtggctcggactgctggctgccatgggagctgttgcaggccaggaggacggttttgagggcactgaggagggctcgccaagagagttcatttacctaaacaggtacaagcgggcgggcgagtcccaggacaagtgcacctacaccttcattgtgccccagcagcgggtcacgggtgccatctgcgtcaactccaaggagcctgaggtgcttctggagaaccgagtgcataagcaggagctagagctgctcaacaatgagctgctcaagcagaagcggcagatcgagacactgcagcagctggtggaggtggacggcggcattgtgagcgaggtgaagctgctgcgcaaggagagccgcaacatgaactcgcgggtcacgcagctctacatgcagctcctgcacgagatcatccgcaagcgggacaacgcgttggagctctcccagctggagaacaggatcctgaaccagacagccgacatgctgcagctggccagcaagtacaaggacctggagcacaagtaccagcacctggccacactggcccacaaccaatcagagatcatcgcgcagcttgaggagcactgccagagggtgccctcggccaggcccgtcccccagccaccccccgctgccccgccccgggtctaccaaccacccacctacaaccgcatcatcaaccagatctctaccaacgagatccagagtgaccagaacctgaaggtgctgccaccccctctgcccactatgcccactctcaccagcctcccatcttccaccgacaagccgtcgggcccatggagagactgcctgcaggccctggaggatggccacgacaccagctccatctacctggtgaagccggagaacaccaaccgcctcatgcaggtgtggtgcgaccagagacacgaccccgggggctggaccgtcatccagagacgcctggatggctctgttaacttcttcaggaactgggagacgtacaagcaagggtttgggaacattgatggcgaatactggctgggcctggagaacatttactggctgacgaaccaaggcaactacaaactcctggtgaccatggaggactggtccggccgcaaagtctttgcagaatacgccagtttccgcctggaacctgagagcgagtattataagctgcggctggggcgctaccatggcaatgcgggtgactcctttacatggcacaacggcaagcagttcaccaccctggacagagatcatgatgtctacacaggaaactgtgcccactaccagaagggaggctggtggtataacgcctgtgcccactccaacctcaacggggtctggtaccgcgggggccattaccggagccgctaccaggacggagtctactgggctgagttccgaggaggctcttactcactcaagaaagtggtgatgatgatccgaccgaaccccaacaccttccactaagccagctccccctcc

SEQ ID NO: 30 - protein, 493 aa, Homo sapiens
MRPLCVTCWWLGLLAAMGAVAGQEDGFEGTEEGSPREFIYLNRYKRAGESQDKCTYTFIVPQQRVTGAICVNSKEPEVLLENRVHKQELELLNNELLKQKRQIETLQQLVEVDGGIVSEVKLLRKESRNMNSRVTQLYMQLLHEIIRKRDNALELSQLENRILNQTADMLQLASKYKDLEHKYQHLATLAHNQSEIIAQLEEHCQRVPSARPVPQPPPAAPPRVYQPPTYNRIINQISTNEIQSDQNLKVLPPPLPTMPTLTSLPSSTDKPSGPWRDCLQALEDGHDTSSIYLVKPENTNRLMQVWCDQRHDPGGWTVIQRRLDGSVNFFRNWETYKQGFGNIDGEYWLGLENIYWLTNQGNYKLLVTMEDWSGRKVFAEYASFRLEPESEYYKLRLGRYHGNAGDSFTWHNGKQFTTLDRDHDVYTGNCAHYQKGGWWYNACAHSNLNGVWYRGGHYRSRYQDGVYWAEFRGGSYSLKKVVMMIRPNPNTFH

SEQ ID NO: 31 - DNA, 25 bp, artificial sequence
  LOX-specific PMO antisense oligonucleotide
ggagcacggtccaggcgaagcgcat

SEQ ID NO: 32 - DNA, 25 bp, artificial sequence
  NOV-specific PMO antisense oligonucleotide
agctcgtgctctgcacactctgcat

SEQ ID NO: 33 - DNA, 25 bp, artificial sequence
  ANGPTL2-specific PMO antisense oligonucleotide
agcatgtcacgcacagtggcctcat